SEQUENCE LISTING (68 sequences)

SEQ ID NO: 1 - DNA, 1433 bp, homo sapiens
ttcggtttcgcttccgcctccagcgcgagccccgccgccgccgagcatggacgaccccgactgcgactccacctgggaggaggacgaggaggatgcggaggacgcggaggacgaggactgcgaggacggcgaggccgccggcgcgagggacgcggacgcaggggacgaggacgaggagtcggaggagccgcgggcggcgcggcccagctcgttccagtccagaatgacagggtccagaaactggcgagccacgagggacatgtgtaggtatcggcacaactatccggatctggtggaacgagactgcaatggggacacgccaaacctgagtttctacagaaatgagatccgcttcctgcccaacggctgtttcattgaggacattcttcagaactggacggacaactatgacctccttgaggacaatcactcctacatccagtggctgtttcctctgcgagaaccaggagtgaactggcatgccaagcccctcacgctcagggaggtcgaggtgtttaaaagctcccaggagatccaggagcggcttgtccgggcctacgagctcatgctgggcttctacgggatccggctggaggaccgaggcacgggcacggtgggccgagcacagaactaccagaagcgcttccagaacctgaactggcgcagccacaacaacctccgcatcacacgcatcctcaagtcgctgggtgagctgggcctcgagcacttccaggcgccgctggtccgcttcttcctggaggagacgctggtgcggcgggagctgccgggggtgcggcagagtgccctggactacttcatgttcgccgtgcgctgccgacaccagcgccgccagctggtgcacttcgcctgggagcacttccggccccgctgcaagttcgtctgggggccccaagacaagctgcggaggttcaagcccagctctctgccccatccgctcgagggctccaggaaggtggaggaggaaggaagccccggggaccccgaccacgaggccagcacccagggtcggacctgtgggccagagcatagcaagggtgggggcagggtggacgaggggccccagccacggagcgtggagccccaggatgcgggacccctggagaggagccagggggatgaggcagggggccacggggaagataggccggagcccttaagccccaaagagagcaagaagaggaagctggagctgagccggcgggagcagccgcccacagagccaggccctcagagtgcctcagaggtggagaagatcgctctgaatttggaggggtgtgccctcagccagggcagcctcaggacggggacccaggaagtgggcggtcaggaccctggggaggcagtgcaaccctgccggcaacccctgggagccagggtggccgacaaggtgaggaaaccggaggaaggtggat

SEQ ID NO: 2 - protein, 477 aa, homo sapiens
SVSLPPPARAPPPPSMDDPDCDSTWEEDEEDAEDAEDEDCEDGEAAGARDADAGDEDEESEEPRAARPSSFQSRMTGSRNWRATRDMCRYRHNYPDLVERDCNGDTPNLSFYRNEIRFLPNGCFIEDILQNWTDNYDLLEDNHSYIQWLFPLREPGVNWHAKPLTLREVEVFKSSQEIQERLVRAYELMLGFYGIRLEDRGTGTVGRAQNYQKRFQNLNWRSHNNLRITRILKSLGELGLEHFQAPLVRFFLEETLVRRELPGVRQSALDYFMFAVRCRHQRRQLVHFAWEHFRPRCKFVWGPQDKLRRFKPSSLPHPLEGSRKVEEEGSPGDPDHEASTQGRTCGPEHSKGGGRVDEGPQPRSVEPQDAGPLERSQGDEAGGHGEDRPEPLSPKESKKRKLELSRREQPPTEPGPQSASEVEKIALNLEGCALSQGSLRTGTQEVGGQDPGEAVQPCRQPLGARVADKVRKPEEGG

SEQ ID NO: 3 - DNA, 978 bp, homo sapiens
cgcggtggctagtggtggtgcccagaccttggcccttgccgggtcccctgccccatcggggcaccccaaggctggacacagtgagaacggggttgaggaggacacagaaggtcgaacggggcccaaagaaggtacccctgggagcccatcggagaccccaggccccagcccagcaggacctgcaggggacgagccagccgagagcccatcggagaccccaggcccccgcccagcaggacctgcaggggacgagccggccgagagcccatcggagaccccaggcccccgcccagcaggacctgcaggggacgagccagccaagaccccatcggagaccccaggccccagcccggcaggacctacaagggatgagccagccgagagcccatcggagaccccaggcccccgcccggcaggacctgcaggggacgagccagccgagagcccatcggagaccccaggcccccgcccggcaggacctgcaggggacgagccagccgagagcccatcggagaccccaggccccagcccggcaggacctacaagggatgagccagccaaggcgggggaggcagcagagttgcaggacgcagaggtggagtcttctgccaagtctgggaagccttaaggaaaggagtgcccgtcggcgtcttggtcctcctgtccctgctgcaggggctggggcctccggagctgctgcgggctcccctcaggctctgcttcgtgacccgtgacccatgacccacagtgctggcctcctgtggggccactatagcagccaccagaagccgcgaggccctcagggaagcccaaggcctgcagaagcctcctggcctggctgtgtcttccccacccagctctcccctgcgcccctgtctttgtaaattgacccttctggagtggggggcggcgggcagggctgcttttcttagtctgatgccaagcaaggccttttctgaataaattcatttgactttg

SEQ ID NO: 4 - protein, 243 aa, homo sapiens
RWLVVVPRPWPLPGPLPHRGTPRLDTVRTGLRRTQKVERGPKKVPLGAHRRPQAPAQQDLQGTSQPRAHRRPQAPAQQDLQGTSRPRAHRRPQAPAQQDLQGTSQPRPHRRPQAPARQDLQGMSQPRAHRRPQAPARQDLQGTSQPRAHRRPQAPARQDLQGTSQPRAHRRPQAPARQDLQGMSQPRRGRQQSCRTQRWSLLPSLGSLKERSARRRLGPPVPAAGAGASGAAAGSPQALLRDP

SEQ ID NO: 5 - DNA, 3400 bp, homo sapiens
gaactgaggagcttgtggagaaaagctatacaccaacaaatcttgttacttcgaatggaaaaagaaaaccagaaacttgaagcaagcagagatgaactccagtccagaaaagttaaattagactatgaagaagttggtgcatgtcagaaagaggtcttaataacttgggataagaagttgttaaactgcagagctaaaatcagatgtgatatggaagatattcatactcttcttaaagaaggagttcccaaaagtcgacgaggagaaatttggcagtttctggctttacagtaccgactcagacacagattgcctaataaacaacagcctcctgacatatcctataaggaacttttgaagcagctcactgctcagcagcatgcgattcttgtggatttaggaaggacgtttcctactcacccttacttttcagtacagcttgggccaggacagctgtcactgtttaacctcctgaaagcctattcattctttgctggacaaagaatgggatactgtcaggggatcagctttgtggctggagtcctgcttctgcacatgagtgaagagcaagcctttgaaatgctgaaattcctcatgtatgacctcggcttccgcaagcagtacagacctgacatgatgtcgctgcagattcaaatgtaccagctgtccaggctccttcatgactatcacagagatctctacaatcaccttgaagaaaatgaaatcagccccagtctttatgctgccccctggttcctcacattgtttgcctctcagttttcattaggatttgtagccagagtttttgatattatttttcttcagggaactgaagttatattcaaggttgcactcagcctactgagcagccaagagacacttataatgggaatgtgagagctttgaaaatattgttgagtttcttaaaaacacgctacctgatatgaatacctctgaaatggaaaaaattattacccaggtttttgagatggatatttctaagcagttgcatgcctatgaggtggaatatcatgtgctacaggatgagcttcaggaatcttcatattcctgtgaggatagtgaaactttggagaagctggagagggccaatagccaactgaaaagacaaaacatggacctcctagaaaaattacaggtagctcatactaaaatccaggccttggaatcaaacctggaaaatcttttgacgagagagaccaaaatgaagtctttaatccggaccctggaacaagaaaaaatggcttatcaaaagacagtggagcaactccggaagctgctgcccgcggatgctctagtcaattgtgacctgttgctgagagacctaaactgcaaccctaacaacaaagccagataggaaataagccataattgaagagcacggctcagcagaaagtgctccttagaatactacagagaggaagagcctgcatgtcgctggcccaaggctggaccctgaagctgatggaaccacctaatactggtgctgagctcctagtcacagcaggtggacctcgtgctcatcagagcatgccaatctaagcccattggacatagtagactggtttttgttgttgctatgacatataaatatatatataaaatgaacatagttcatgctttcagataaaatgagtagatgtatatttagattaatttttttagtcagaacttcatgaaatccacaccaaaggaaaggtaaactgaaatttcccttggacatatgtgaaatctttttgtctttatagtgaaacaaagccagagcatctttgtatattgcaatatacttgaaaaaaatgaatgtatttttttctccaaagaacagcatgtttcactcaatggtgaaaaggtggaaacatttatgttaactttatgtgttctgtcttgatatctactgacattgtctatatgaggaaaatgattactggtcatgctcctgtgattttttgggaaggtagggtcatttctccctgcctgctttgtgccaactagcatgttgcatctactgcattatgaatctggtggcttacttttaaacatactaaaaacagtaggacttggctgaatctacccccaggtaaaggagaatgttgcttattttttagcaaactaacagccttattctcaactaaaatatcacacctgaaaaatttaattttttggtgccacagtcaccaaatgacaaggatttgccactttcccaccaaattgtgagtgcttgtaatttaggtctctctaccttaaattcagtataaggaaacgtaattatgattgattttttccaaagatgacaagctgtgttgaaatacatttttcttttgaccaattgacagaatctaataagctttaataatcttccccttttatgtgaaaagttttgagaactgtgaaatgtttaggaacaaactgttgaaatccattggaagggaaaaaagaaagtggtaccagtgttaccagctcaactaaaacctgcaattgtgcatttcaacttttcacttcctcagcatacaaatagctcattagaagacattcacgcatggtgggtataggcaaggaaagtaattttcaaagtacatttgcagttctctttttcagagatgattctatgatagcgcctctgaaagttgatgcagcattttcgcctttccaaaaagtatttatcctcactgctttttgcagtacttgtattttcacagatggattatctggggtaattttcttcaaagggagtttgttatacacagtgaaaatgtattatagagtagaatagtaaagctctaggggtttcagaaagctttgatgaacagatgacaaacatctgaaaccccctccgcactgttacccagtgtgtatataatgacttgttatagctcagtgtgcccttgaatccatacagtttcttaaaagacaataaaatcttattaataaagttaatgtaacttctaagttctagaaaatgctgattctgtctgccccattcaattgggggctactaattgatttgttgcttggatttcctgagaatttctctatttgtaggaggggttttttctttttacggtctgttgatgacaattactttatgggtgtgatgcaccgatggtagccaaggaatctgttggggaagttcggaaagaaaccttttctttcttttattcagtttaaagtaaactttatcctggatgtttagaatcaacattaagagttatattatggtgttcagagattaagctgacttggatacaatattttcttttgaaaatgaattttctttttcatttgtgatttttaaaaaatgttgcaccagttatgcttcatgcatcgttacatcttcatcaggttaatgtaatgtctagttcctttgcaataaatatattgctgc

SEQ ID NO: 6 - protein, 366 aa, homo sapiens
MTVRNIASICNMGTNASALEKDIGPEQFPINEHYFGLVNFGNTCYCNSVLQALYFCRPFRENVLAYKAQQKKKENLLTCLADLFHSIATQKKKVGVIPPKKFISRLRKENDLFDNYMQQDAHEFLNYLLNTIADILQEEKKQEKQNGKLKNGNMNEPAENNKPELTWVHEIFQGTLTNETRCLNCETVSSKDEDFLDLSVDVEQNTSITHCLRDFSNTETLCSEQKYYCETCCSKQEAQKRMRVKKLPMILALHLKRFKYMEQLHRYTKLSYRVVFPLELRLFNTSSDAVNLDRMYDLVAVVVHCGSGPNRGHYITIVKSHGFWLLFDDDIVEKIDAQAIEEFYGLTSDISKNSESGYILFYQSRE

SEQ ID NO: 7 - DNA, 3207 bp, homo sapiens
gagaactacaaaaaagaaaaagcagaaaatgaaaaaatacaaaatgagcagcttgagaaacttcaagaacaagttacagatttgcgatcacaaaataccaaaatttctacccagctagattttgcttctaaacgttatgaaatgctgccagataatgttgaaggatatcgtcgagaaataacatcacttcctgagagaaatcagaaactcactgccacaactccaaagccagaacagattatccatacgatgactccgatttgagaggagccaatgagaagctagctgtcgccgaagtttgagccgaaaatttgaagaaggaaaaggaaatgcttaaattgtctgaagttcgtctttctcagcaaagagagtctttgttagctgaacaaagggggcaaaacttactgctaactaatctgcaaacaattcagggaatactggagcgatctgaaacagaaaccaaacaaaggcttagtagccagatagaaaaactggaacatgagatctctcatctaaagaagaagttggaaaatgaggtggaacaaaggcatacacttactagaaatctagatgttcaacttttagatacaaagagacaactggatacagagacaaatcttcatcttaacacaaaagaactattaaaaaatgctcaaaaagaaattgccacattgaaacagcacctcagtaatatggaagtccaagttgcttctcagtcttcacagagaactggtaaaggtcggcctagcaacaaagaagatgtggatgatcttgtgagtctgctaagacagacagaagagcaggtgaatgacttaaaggagagactcaaaaaaaacaagtacgagcaatgtggaacaatatcaagcaatggttactagtttagaagaatccctgaacaaggaaaaacaggtgacagaagaagtgcgtaagaatattgaagttcgtttaaaagagtcagctgaatttcagacacagttggaaaagaagttgatggaagtagagaaggaaaaacaagaacttcaggatgataaaagaagagccatagagagcatggaacaacagttatctgaattgaagaaaacactttcctagtgttcagaatgaagtacaagaagctcttcagagagcaagcacagctttaagtaatgagcagcaagccagacgtgactgtcaggaacaagctaaaatagctgtggaagctcagaataagtatgagagagaattgatgctgcatgctgctgatgttgaagctctacaagctgcgaaggagcaggtttcaaaaatggcatcagtccgtcagcatttggaagaaacaacacagaaagcagaatcacagttgttggagtgtaaagcatcttgggaggaaagagagagaatgttaaaggatgaagtttccaaatgtgtatgtcgctgtgaagatctggagaaacaaaacagattacttcatgatcagatcgaaaaattaagtgacaaggtcgttgcctctgtgaaggaaggtgtacaaggtcccactgaatgtatctctcagtgaagaaggaaaatctcaagaacaaattttggaaattctcagatttatacgacgagaaaaagaaattgctgaaactaggtttgaggtggctcaggttgagagtctgcgttatcgacaaagggttgaacttttagaaagagagctgcaggaactgcaagatagtctaaatgctgaaagggagaaagtccaggtaactgcaaaaacaatggctcagcatgaagaactgatgaagaaaactgaaacaatgaatgtagttatggagaccaataaaatgctaagagaagagaaggagagactagaacaggatctacagcaaatgcaagcaaaggtgaggaaactggagttagatattttacccttacaagaagcaaatgctgagctgagtgagaaaagcggtatgttgcaggcagagaagaagctcttagaagaggatgtcaaacgttggaaagcacgtaaccagcatctagtaagtcaacagaaagatccagatacagaagaatatcggaagctcctttctgaaaaggaagttcatactaagcgtattcaacaattgacagaagaaattggtagacttaaagctgaaattgcaagatcaaatgcatctttgactaacaaccagaacttaattcagagtctgaaggaagatctaaataaagtaagaactgaaaaggaaaccatccagaaggacttagatgccaaaataattgatatccaagaaaaagtcaaaactattactcaagttaagaaaattggacgtaggtacaagactcaatatgaagaacttaaagcacaacaggataaggttatggagacatcggctcagtcttctggagaccatcaggagcagcatgtttcagtccaggaaatgcaggaactcaaagaaacgctcaaccaagctgaaacaaaatcaaaatcacttgaaagtcaagtagagaatttgcagaagacattatttgaaaaagagacagaagcaagaaatctccaggaacagactgtgcaacttcagtctgaactttcacgactttgtcaggattttcaagatagaaccacacaggaggagcagctccgacaacagataactaaaaaaaaaaaactcgtgccgaattcggcacgagctcccagccaaattgaaagccggaccccaggccgccgcgttgccgcccggcctccccgccagcgcgccaccatgggcagtcccggtttccccttgtaaagatggcggtgagggatcgctgcaacctttagattaatgactctccgaaacatcgcctcccatctgtaatatgggcacccaatgcttttgttttggaaaaagacattggtccagagcagtttccaatcaatgaacactatttcggattggtcaattttggaaacacatgctactgtaactccgtgcttcaggcattgtacttctgccgtccattccgggagaatgtgttggcatacaaggcccagcaaaagaagaaggaaaacttgctgacgtgcctggcggaccttttccacagcattgccacacagaagaagaaggttggcgtcatcccaccaaagaagttcatttcaaggctgagaaaagagaatgatctctttgataactacatgcagcaggatgctcatgaatttttaaattatttgctaaacactattgcggacatccttcaggaggagaagaaacaggg

SEQ ID NO: 8 - DNA, 3683 bp, homo sapiens
aacagatggaaaaatagtacagtatgaatgtgagggggatacttgccaggaagagaaaatagatgccttacagttagagtattcatatttactaacaagccagctggaatctcagcgaatctactgggaaaacaagatagttcggatagagaaggacacagcagaggaaattaacaacatgaagaccaagtttaaagaaacaattgagaagtgtgataatctagagcacaaactaaatgatctcctaaaagaaaagcagtctgtggaaagaaagtgcactcagctaaacacaaaagtggccaaactcaccaacgagctcaaagaggagcaggaaatgaacaagtgtttgcgagccaaccaagtcctcctgcagaacaagctaaaagaggaggagagggtgctgaaggagacctgtgaccaaaaagatctgcagatcaccgagatccaggagcagctgcgtgacgtcatgttctacctggagacacagcagaagatcaaccatctgcctgccgagacccggcaggaaatccaggagggacagatcaacatcgccatggcctcggcctcgagccctgcctcttcggggggcagtgggaagttgccctccaggaagggccgcagcaagaggggcaagtgaccttcagagcaacagacatccctgagactgttctccctgacactgtgagagtgtgctgggaccttcagctaaatgtgagggtgggccctaataagtacaagtgaggatcaagccacagttgtttggctctttcatttgctagtgtgtgatgtagtgaatgtaaagggtgctgactggagagctgatagaaaggcgctgcgttcgaaaaggtcttaagagttcactaacctcacattctaatgaccattttgccttcctgcttggtagaagccccaactctgctgtgcatttttccattgtatttatggagttggcgtatttgacattcagttctggggtaggtttaagatgttaagttatttcttgtaacctcaaaggtaaggttatctagcactaaagcaccaaacctctctgagggcataacagctgctttaaagagaggtttccattggctattaaggagttatgaaaactccctagcaatagtgtcatatcattatcatctcccccttcctctggggagtggaagaattgcttgaatgttatctgaaaagaggcctggtagtaaaccaggccctggctctttaccagcagtcatctcttcttgctctggggccagccaggaaaaacaaacaacccggggcacattgggtagactcagtgtaggaaaaatggtggcagctccactgtttatttttggtgacttcgtacgtcattatgaaccgcaattaaggaggaggcttaatggctgttcccaaactcaaatctcagagtgggtatcctagcatctagcaagactgagtggggagatttctcatccgtgtgaaaatgtagagtgaggcctctgactagctaattgtgtattttgttgggtttagtattttctaaatgtttacaaaatattgggctgcatgttcaggttgcagctagagggagcttgggcagattttcaattacgctttcaagatataaccaaaagctgtttctaaatcctaaaattagaatttcaacagagccccctttagaacagtcatataacgcttgtgtgggccaacagaggggctgtgtactctctctggaaccataaatgtcaaataatttataacctgcagtaattgagcaaacttaaaataagacctgtgttggaatttagtttcttgaagaggtagagggataggttagtaagatgtattgttaaacaacaggttttagtttttgctttataattagccacaggttttcaaatgatcacatttcagaataggtttttagcctgtaattaggcctcatcccctttgacctaaatgtcttacatgttacttgttagcacatcaactgtatcactaatcaccatctgtttttgtgggatgtgctgcagcatttcccaaaaaactttacgtgtaatgttgcaaaatgaatgtactcagacattcttaatttttacttagggcagaccaactctttgagtctctcttggacttatatatacagatatcttaagagtgggaatgtaaagcataacctaattctctttcctatagagattctattttatttaaaatctatttttacactagttagaatcctgctgttttggatcaagtacttgtcttgcatgtctgaccttgcagaagctggggtggatcatagcatactaatgaagagaattagaagtagtttacaaagctcgctcactcctcatttctctgtgatcccttctatccagtggccccaccaccacctgggaaaacagatttttcagtacaggtgggataaatgctctgaaaggctgtgcccagaggaatgagcaaataggcaagtgtttccaaactacttggaggtttacaaaaaatatgtcccagaaaaaaaaaaactcgtgccgaattcggcacgagggaggacctgactcccctcacctttggggtgcaggaactcaacctgactggctccttctggaatgactcctttgccaggctctcactgacctatgaacgactctttggtaccacagtgacattcaagttcattctggccaaccgcctctacccagtgtctgcccggcactggtttaccatggagcgcctcgaagtccacagcaatggctccgtcgcctacttcaatgcttcccaggtcacagggcccagcatctactccttccactgcgagtatgtcagcagcctgagcaagaagggtagtctcctcgtggcccgcacgcagccctctccctggcagatgatgcttcaggacttccagatccaggctttcaacgtaatgggggagcagttctcctacgccagcgactgtgccagcttcttctcccccggcatctggatggggctgctcacctccctgttcatgctcttcatcttcacctatggcctgcacatgatcctcagcctcaagaccatggatcgctttgatgaccacaagggccccactatttctttgacccagattgtgtgaccctgtgccagtgggggggttgagggtgggacggtgtccgtgttgttgctttcccaccctgcagcgcactggactgaagagcttccctcttcctactgcagcatgaactgcaagctcccctcagcccatcttgctccctcttcagcccgctgaggagctttcttgggctgcccccatctctcccaacaaggtgtacatattctgcgtagatgctagaccaaccagcttcccagggttcgtcgctgtgaggcgtaagggacatgaattctagggtctcctttctccttatttattcttgtggctacatcatccctggctgtggatagtgcttttgtgtagcaaatgctccctccttaaggttatagggctccctgagtttgggagtgtggaagtactacttaactgtctgtcctgcttggctgtcgttatcgttttctggtgatgttgtgctaacaataagaagtacacgggtttatttctgtggcctgagaaggaagggacctccacgacaggtgggctgggtgcgatcgccggctgtttggcatgttcccaccgggagtgccgggcaggagcatggggtgct

SEQ ID NO: 9 - DNA, 3505 bp, homo sapiens
aaatatagatctcgacctcgaaattgtacagtctttgcagcatggtcatggaggatggactgatggaatgtttgagactttaactacaactggaactgtttgtggcattgatgaagatcatgacattgtagtacagtatccaagtggcaataggtggaccttcaatcctgctgttctcactaaagcgaacattgtccgaagtggagatgctgctcagggtgcagaaggaggcacctcgcagtttcaagtgggtgatcttgtacaagtttgttatgacctggaacgaattaaacttctacaaagaggacatggagaatgggctgaagcgatgcttccaactttaggtaaagttggccgagtacaacagatttattcagacagtgatttaaaggtggaagtttgtggaacatcttggacatacaatccagcagcagtttccaaggtggcatctgcaggatcagccattagcaatgcatctggtgaaagactctcacaactcctgaagaaattatttgaaacccaagaatctggtgacctcaatgaagaattagttaaggctgctgccaatggagatgttgctaaagtggaagatttgcttaaaagaccagatgtggatgtaaatgggcaatgtgctggccacacagctatgcaagctgctagtcagaatggacatgttgacattttgaagttacttttgaagcaaaacgtggatgtcgaagcagaggataaagatggtgatagagcagttcaccatgcagcttttggagatgaaggcgctgttatagaagtactacatcgaggtagtgctgatttgaatgctcgaaacaagcgccgacagacaccacttcatattgctgtcaataaaggtcatcttcaagttgtgaagactttattggactttggctgtcatcccagtctccaggattctgaaggtgatacccctcttcatgatgcaataagtaagaaacgtgatgatatcctagcagttcttttggaagctggagcagatgttaccatcacaaacaataatggatttaatgctctgcatcatgctgcactaaggggaaatcccagtgcaatgcgtgttttactatctaaattaccaagaccatggattgtggatgagaagaaagatgatggttatactgccttacatctggctgcccttaataatcacgtagaagtggctgaactgttggtacatcagggtaatgcaaacctggatatccagaatgtgaaccaacaaactgccctacaccttgctgttgaacgacagcatacccagattgttaggcttttggtccgtgcaggtgccaagcttgatattcaggataaggatggggatactcctttgcatgaagctctaaggcatcacactttgtctcagctacgtcagctccaagatatgcaagatgtggggaaggtggatgctgcctgggagccatccaaaaacacgttaataatgggacttggtacccagggggcagagaagaagagtgcagcatctattgcctgtttcttggcagccaatggtgctgacctgagcattcgaaataagaagggtcaatcgccacttgatctctgtcctgatccgaatctctgcaaagcactggcaaagtgtcataaggaaaaagtcagtggtcaagtgggttctcggagtccttctatgattagtaatgattctgaaaccttagaagagtgtatggtgtgctcagatatgaagagagatactctttttggtccatgtggacatattgctacctgttctttatgttctccacgtgtcaagaaatgcctcatctgtaaagaacaggttcaatccaggacaaagattgaagaatgtgtggtatgctctgacaagaaagcagctgttctttttcaaccctgtggccacatgtgtgcttgtgagaactgtgctaacctgatgaaaaagtgtgtgcagtgtcgagcagtagttgaacgaagagtgcctttcattatgtgctgtggagggaaaagttcagaagatgccactgatgatatctcaagtgggaatattccagtattacaaaaggacaaggataataccaatgtcaatgcagatgtgcaaaagttgcagcaacagttacaagacattaaagagcagacaatgtgccctgtgtgtctagatcgtctgaagaatatgattttcctttgtggtcacggaacctgtcaactctgtggagaccgcatgagtgaatgtcctatctgtcgcaaggctattgaacgaaggattcttttgtattaactaagacacatggtgtattttgttagctaatgtatctagtcatgagatcttaataggcttttgatctagttggaagttctgatgagttaatttctaatatcatagtttctttactagagtataattgggctgtaaatgtaccagaacaaaaaaccctacaaaatggtgttggaaattgtgttttttgtttttgttttaaatttgaaacatcaaattcatgtaactcataggataatttacctttggcttctaagaggaaagtcctttaaggatatccttttttaaaaaattgcatttttctcttataatttgtaaatttgttggatctcaaaagacataattctttgtgatcagttatccttcatttcatcgtggttttacacagtgagttgataacaggttctctgagaagtcatgcatcaaataaaagaggcaggtcaaacaattatgtcacatggtaaattataaaatgacagtacaagttccagatagttaagggaataccgaagggatgattctttttttaagataacaggaagttacccacatgtttgtttctgaattcttagagtaaatggaagcatagaatgagggaataatgactttgcatttctcttgttttctagattcaaaaggaacattgtttaacttgaatcagattaccagtttcaaggtgactgatagacaagaaaaggaaaaataagcaataatagtgggcaactgaagagaaaaaaaaaacgagtatctattaactggccactaacagttgcctttcttacattaatttatacactattttgttcagccagtgtttttaaaaaaaatctatgaaaagtgtacttccggttttctgtgattacttatctgggcttgatctgaccagtgaaatgacattgccctatttggacctctgaggttctatttagctttgcagatgtacatagtatcccagtgatctgcaaaattaatgccttttccaagaaaaaatcttttcttctctgtatcagttaattctgacagtgttagtgattctgtcttcattataggccttatttccattatctctttctttatagtattttttgttataaagaaaacagtctttctgtgtatacctacggatgagggtattatttaaactgccaacaatatccaagacatggtcaataacctaattataaatactttagaaagagtgaccaggacatgtatagaaatgtctgcttacctgtagacttt

SEQ ID NO: 10 - protein, 1168 aa, homo sapiens
NIDLDLEIVQSLQHGHGGWTDGMFETLTTTGTVCGIDEDHDIVVQYPSGNRWTFNPAVLTKANIVRSGDAAQGAEGGTSQFQVGDLVQVCYDLERIKLLQRGHGEWAEAMLPTLGKVGRVQQIYSDSDLKVEVCGTSWTYNPAAVSKVASAGSAISNASGERLSQLLKKLFETQESGDLNEELVKAAANGDVAKVEDLLKRPDVDVNGQCAGHTAMQAASQNGHVDILKLLLKQNVDVEAEDKDGDRAVHHAAFGDEGAVIEVLHRGSADLNARNKRRQTPLHIAVNKGHLQVVKTLLDFGCHPSLQDSEGDTPLHDAISKKRDDILAVLLEAGADVTITNNNGFNALHHAALRGNPSAMRVLLSKLPRPWIVDEKKDDGYTALHLAALNNHVEVAELLVHQGNANLDIQNVNQQTALHLAVERQHTQIVRLLVRAGAKLDIQDKDGDTPLHEALRHHTLSQLRQLQDMQDVGKVDAAWEPSKNTLIMGLGTQGAEKKSAASIACFLAANGADLSIRNKKGQSPLDLCPDPNLCKALAKCHKEKVSGQVGSRSPSMISNDSETLEECMVCSDMKRDTLFGPCGHIATCSLCSPRVKKCLICKEQVQSRTKIEECVVCSDKKAAVLFQPCGHMCACENCANLMKKCVQCRAVVERRVPFIMCCGGKSSEDATDDISSGNIPVLQKDKDNTNVNADVQKLQQQLQDIKEQTMCPVCLDRLKNMIFLCGHGTCQLCGDRMSECPICRKAIERRILLYZLRHMVYFVSZCIZSZDLNRLLIZLEVLMSZFLISZFLYZSIIGLZMYQNKKPYKMVLEIVFFVFVLNLKHQIHVTHRIIYLWLLRGKSFKDILFZKIAFFSYNLZICWISKDIILCDQLSFISSWFYTVSZZQVLZEVMHQIKEAGQTIMSHGKLZNDSTSSRZLREYRRDDSFFKITGSYPHVCFZILRVNGSIEZGNNDFAFLLFSRFKRNIVZLESDYQFQGDZZTRKGKISNNSGQLKRKKKRVSINWPLTVAFLTLIYTLFCSASVFKKNLZKVYFRFSVITYLGLIZPVKZHCPIWTSEVLFSFADVHSIPVICKINAFSKKKSFLLCISZFZQCZZFCLHYRPYFHYLFLYSIFCYKENSLSVYTYGZGYYLNCQQYPRHGQZPNYKYFRKSDQDMYRNVCLPVDF

SEQ ID NO: 11 - DNA, 1246 bp, homo sapiens
cggcacgagctcgtgccgggcaggcctgtgcctatccctgctgtccccagggtgggccccgggggtcaggagctccagaagggccagctgggcatattctgagattggccatcagcccccatttctgctgcaaacctggtcagagccagtgttccctccatgggacctaaagacagtgccaagtgcctgcaccgtggaccacagccgagccactgggcagccggtgatggtcccacgcaggagcgctgtggaccccgctctctgggcagccctgtcctaggcctggacacctgcagagcctgggaccacgtggatgggcagatcctgggccagctgcggcccctgacagaggaggaagaggaggagggcgccggggccaccttgtccagggggcctgccttccccggcatgggctctgaggagttgcgtctggcctccttctatgactggccgctgactgctgaggtgccacccgagctgctggctgctgccggcttcttccacacaggccatcaggacaaggtgaggtgcttcttctgctatgggggcctgcagagctggaagcgcggggacgacccctggacggagcatgccaagtggttccccagctgtcagttcctgctccggtcaaaaggaagagactttgtccacagtgtgcaggagactcactcccagctgctgggctcctgggacccgtgggaagaaccggaagacgcagcccctgtggccccctccgtccctgcctctgggtaccctgagctgcccacacccaggagagaggtccagtctgaaagtgcccaggagccaggagccagggatgtggaggcgcagctgcggcggctgcaggaggagaggacgtgcaaggtgtgcctggaccgcgccgtgtccatcgtctttgtgccgtgcggccacctggtctgtgctgagtgtgcccccggcctgcagctgtgccccatctgcagagcccccgtccgcagccgcgtgcgcaccttcctgtcctaggccaggtgccatggccggccaggtgggctgcagagtgggctccctgcccctctctgcctgttctggactgtgttctgggcctgctgaggatggcagagctggtgtccatccagcactgaccagccctgattccccgaccaccgcccagggtggagaaggaggcccttgcttggcgtgggggatggcttaactgtacctgtttggatgcttctgaatagaaataaagtgggttttccctggaggt

SEQ ID NO: 12 - protein, 309 aa, homo sapiens
MGPKDSAKCLHRGPQPSHWAAGDGPTQERCGPRSLGSPVLGLDTCRAWDHVDGQILGQLRPLTEEEEEEGAGATLSRGPAFPGMGSEELRLASFYDWPLTAEVPPELLAAAGFFHTGHQDKVRCFFCYGGLQSWKRGDDPWTEHAKWFPSCQFLLRSKGRDFVHSVQETHSQLLGSWDPWEEPEDAAPVAPSVPASGYPELPTPRREVQSESAQEPGARDVEAQLRRLQEERTCKVCLDRAVSIVFVPCGHLVCAECAPGLQLCPICRAPVRSRVRTFLSZARCHGRPGGLQSGLPAPLCLFWTVFWAC

SEQ ID NO: 13 - DNA, 3478 bp, homo sapiens
gaactgaggagcttgtggagaaaagctatacaccaacaaatcttgttacttcgaatggaaaaagaaaaccagaaacttgaagcaagcagagatgaactccagtccagaaaagttaaattagactatgaagaagttggtgcatgtcagaaagaggtcttaataacttgggataagaagttgttaaactgcagagctaaaatcagatgtgatatggaagatattcatactcttcttaaagaaggagttcccaaaagtcgacgaggagaaatttggcagtttctggctttacagtaccgactcagacacagattgcctaataaacaacagcctcctgacatatcctataaggaacttttgaagcagctcactgctcagcagcatgcgattcttgtggatttaggaaggacgtttcctactcacccttacttttcagtacagcttgggccaggacagctgtcactgtttaacctcctgaaagcctattcattctttgctggacaaagaatgggatactgtcaggggatcagctttgtggctggagtcctgcttctgcacatgagtgaagagcaagcctttgaaatgctgaaattcctcatgtatgacctcggcttccgcaagcagtacagacctgacatgatgtcgctgcagattcaaatgtaccagctgtccaggctccttcatgactatcacagagatctctacaatcaccttgaagaaaatgaaatcagccccagtctttatgctgccccctggttcctcacattgtttgcctctcagttttcattaggatttgtagccagagtttttgatattatttttcttcagggaactgaagttatattcaaggttgcactcagcctactgagcagccaagagacacttataatgggaatgtgagagctttgaaaatattgttgagtttcttaaaaacacgctacctgatatgaatacctctgaaatggaaaaaattattacccaggtttttgagatggatatttctaagcagttgcatgcctatgaggtggaatatcatgtgctacaggatgagcttcaggaatcttcatattcctgtgaggatagtgaaactttggagaagctggagagggccaatagccaactgaaaagacaaaacatggacctcctagaaaaattacaggtagctcatactaaaatccaggccttggaatcaaacctggaaaatcttttgacgagagagaccaaaatgaagtctttaatccggaccctggaacaagaaaaaatggcttatcaaaagacagtggagcaactccggaagctgctgcccgcggatgctctagtcaattgtgacctgttgctgagagacctaaactgcaaccctaacaacaaagccagataggaaataagccataattgaagagcacggctcagcagaaagtgctccttagaatactacagagaggaagagcctgcatgtcgctggcccaaggctggaccctgaagctgatggaaccacctaatactggtgctgagctcctagtcacagcaggtggacctcgtgctcatcagagcatgccaatctaagcccattggacatagtagactggtttttgttgttgctatgacatataaatatatatataaaatgaacatagttcatgctttcagataaaatgagtagatgtatatttagattaatttttttagtcagaacttcatgaaatccacaccaaaggaaaggtaaactgaaatttcccttggacatatgtgaaatctttttgtctttatagtgaaacaaagccagagcatctttgtatattgcaatatacttgaaaaaaatgaatgtatttttttctccaaagaacagcatgtttcactcaatggtgaaaaggtggaaacatttatgttaactttatgtgttctgtcttgatatctactgacattgtctatatgaggaaaatgattactggtcatgctcctgtgattttttgggaaggtagggtcatttctccctgcctgctttgtgccaactagcatgttgcatctactgcattatgaatctggtggcttacttttaaacatactaaaaacagtaggacttggctgaatctacccccaggtaaaggagaatgttgcttattttttagcaaactaacagccttattctcaactaaaatatcacacctgaaaaatttaatttaggacctaaaatgtctagattagctttctgctttttttatttgaataactcattcagttgtgaatgaattcctctttatttggtgccacagtcaccaaatgacaaggatttgccactttcccaccaaattgtgagtgcttgtaatttaggtctctctaccttaaattcagtataaggaaacgtaattatgattgattttttccaaagatgacaagctgtgttgaaatacatttttcttttgaccaattgacagaatctaataagctttaataatcttccccttttatgtgaaaagttttgagaactgtgaaatgtttaggaacaaactgttgaaatccattggaagggaaaaaagaaagtggtaccagtgttaccagctcaactaaaacctgcaattgtgcatttcaacttttcacttcctcagcatacaaatagctcattagaagacattcacgcatggtgggtataggcaaggaaagtaattttcaaagtacatttgcagttctctttttcagagatgattctatgatagcgcctctgaaagttgatgcagcattttcgcctttccaaaaagtatttatcctcactgctttttgcagtacttgtattttcacagatggattatctggggtaattttcttcaaagggagtttgttatacacagtgaaaatgtattatagagtagaatagtaaagctctaggggtttcagaaagctttgatgaacagatgacaaacatctgaaaccccctccgcactgttacccagtgtgtatataatgacttgttatagctcagtgtgcccttgaatccatacagtttcttaaaagacaataaaatcttattaataaagttaatgtaacttctaagttctagaaaatgctgattctgtctgccccattcaattgggggctactaattgatttgttgcttggatttcctgagaatttctctatttgtaggaggggttttttctttttacggtctgttgatgacaattactttatgggtgtgatgcaccgatggtagccaaggaatctgttggggaagttcggaaagaaaccttttctttcttttattcagtttaaagtaaactttatcctggatgtttagaatcaacattaagagttatattatggtgttcagagattaagctgacttggatacaatattttcttttgaaaatgaattttctttttcatttgtgatttttaaaaaatgttgcaccagttatgcttcatgcatcgttacatcttcatcaggttaatgtaatgtctagttcctttgcaataaatatattgctgc

SEQ ID NO: 14 - DNA, 1956 bp, homo sapiens
gctgactggctagcacaaaacaaccctcctcaaatgctatgggaaagaacagaagaggattctaaaagcattaaaagtgatgttccagtgtacttgaaaaggttgaaaggaaataaacatgatgatggtacgcaaagtgattcagagaacgctggggctcacaggcgctgtagcaaacgtgcaactcttgaggaacacttaagacgccaccattcagaacacaaaaagctacagaaggtccaggctactgaaaagcatcaagaccaagctgttactagctctgcgcatcacagaggggggcatggtgttccacatgggaaattgttaaaacagaaatcagaggagccatcggtgtcaatacccttcctacaaactgcattattaagaagttcagggagtcttgggcacagaccaagccaggagatggataaaatgttaaaaaatcaagcaacttctgctacttctgaaaaggataatgatgatgaccaaagtgacaagggtacttataccattgagttagagaatcccaacagtgaggaagtggaagcaagaaaaatgattgacaaggtgtttggagtagatgacaatcaggattataataggcctgttatcaacgaaaaacataaagatctaataaaagattgggctctcagttctgctgcagcagtaatggaagaaagaaaaccactgactacatctggatttcaccactcagaggaaggcacatcttcatctggaagcaaacgttgggtttcacagtgggctagtttggctgccaatcatacaaggcatatcaagaagaaaggataatggaattttctgcacctcttcctttagagaatgagacagagatcagtgagtctggcatgacagtgagaagtactggctctgcaacttccttggctagccagggagagagaaggagacgaactcttccccagcttccaaatgaagaaaagtctcttgagagccacagagcaaaggttgtaacacagaggtcagagataggagaaaaacaagacacagaacttcaggagaaagaaacacctacacaggtataccagaaagataaacaagatgctgacagacccttgagtaaaatgaacagggcagtaaatggagagactctcaaaactggtggagataataaaaccctacttcacttaggcagctctgctcctggaaaagagaaaagtgaaactgataaggaaacttctttggtaaagcaaacattagcaaaacttcaacaacaagaacaaagggaggaggctcagtggacacctactaaattgtcttccaaaaatgtttcaggtcagacagataaatgtagggaggaaacttttaaacaagaatcacaacctccagaaaaaaattcaggacattctacaagcaaaggagacagagtggcacaaagtgagagcaagagaagaaaagctgaggaaattctgaaaagtcagactccaaagggaggagacaagaaggaatcctccaagtcattagtgcgacaagggagcttcactatagaaaaacccagcccaaacatacccatagaacttattccccatataaataaacagacttcctctactccttcttctttagcattaacatctgcaagtagaatacgagaaagaagtgagtctttggatcctgattctagtatggacacaacccttattctaaaagacacagaagcagtaatggcttttctagaagctaaactacgtgaagataataaaactgatgaaggaccagatactcccagttataatagagacaattctatttcaccagaatctgatgtagatacagctagtacaatcagtctggttactggagaaactgaaagaaagtcaacccaaaagcgaaagagtttcactagcctctataaagataggtgttccacaggttctccttccaaagatgttacaaaatcatcatcttcaggtgctaggg

SEQ ID NO: 15 - DNA, 2417 bp, homo sapiens
ggatgacgtagctttgccaaagacttagaagctaagcagaaaatgagcttaacatcctggtttttggtgagcagtggaggcactcgccacaggctgccacgagaaatgatttttgttggaagagatgactgtgagctcatgttgcagtctcgtagtgtggataagcaacacgctgtcatcaactatgatgcgtctacggatgagcatttagtgaaggatttgggcagcctcaatgggacttttgtgaatgatgtaaggattccggaacagacttatatcaccttgaaacttgaagataagctgagatttggatatgatacaaatcttttcactgtagtacaaggagaaatgagggtccctgaagaagctcttaagcatgagaagtttaccattcagcttcagttgtcccaaaaatcttcagaatcagaattatccaaatctgcaagtgccaaaagcatagattcaaaggtagcagacgctgctactgaagtgcagcacaaaactactgaagcactgaaatccgaggaaaaagccatggatatttctgctatgccccgtggtactccattatatgggcagccgtcatggtggggggatgatgaggtggatgaaaaaagagctttcaagacaaatggcaaacctgaaaaaaaaaaccatgaagctggaacatcagggtgcagcatagatgccaagcaagttgaggaacaatctgcagctgcaaatgaagaagtactttttcctttctgtagggaaccaagttattttgaaatccctacaaaagaattccagcaaccatcacaaataacagaaagcactattcatgaaatcccaacaaaagacacgccaagttcccatataacaggtgcagggcatgcttcatttaccattgaatttgatgacagtaccccagggaaggtaactattagagaccatgtgacaaagtttacttctgatcagcgccacaagtccaagaagtcttctcctggaactcaagacttgctggggattcaaacaggaatgatggcacccgaaaacaaagttgctgactggctagcacaaaacaaccctcctcaaatgctatgggaaagaacagaagaggattctaaaagcattaaaagtgatgttccagtgtacttgaaaaggttgaaaggaaataaacatgatgatggtacgcaaagtgattcagagaacgctggggctcacaggcgctgtagcaaacgtgcaactcttgaggaacacttaagacgccaccattcagaacacaaaaagctacagaaggtccaggctactgaaaagcatcaagaccaagctgttgtgtttggagtagatgacaatcaggattataataggcctgttatcaacgaaaaacataaagatctaataaaagattgggctctcagttctgctgcagcagtaatggaagaaagaaaaccactgactacatctggatttcaccactcagaggaaggcacatcttcatctggaagcaaacgttgggtttcacagtgggctagtttggctgccaatcatacaaggcatgatcaagaagaaaggataatggaattttctgcacctcttcctttagagaatgagacagagatcagtgagtctggcatgacagtgagaagtactggctctgcaacttccttggctagccagggagagagaaggagacgaactcttccccagcttccaaatgaagaaaagtctcttgagagccacagagcaaaggttgtaacacagaggtcagagataggagaaaaacaagacacagaacttcaggagaaagaaacacctacacaggtataccagaaagataaacaagatgctgacagacccttgagtaaaatgaacagggcagtaaatggagagactctcaaaactggtggagataataaaaccctacttcacttaggcagctctgctcctggaaaagagaaaagtgaaactgataaggaaacttctttggtaaagcaaacattagcaaaacttcaacaacaagaacaaagggaggaggctcagtggacacctactaaattgtcttccaaaaatgtttcaggtcagacagataaatgtagggaggaaacttttaaacaagaatcacaacctccagaaaaaaattcaggacattctacaagcaaaggagacagagtggcacaaagtgagagcaagagaagaaaagctgaggaaattctgaaaagtcagactccaaagggaggagacaagaaggaatcctccaagtcattagtgcgacaagggagcttcactatagaaaaacccagcccaaacatacccatagaacttattccccatataaataaacagacttcctctactccttcttctttagcattaacatctgcaagtagaatacgagaaagaagtgagtctttggatcctgattctagtatggacac

SEQ ID NO: 16 - DNA, 3617 bp, homo sapiens
aaaaggaggaggcttaatcaatattggggggggggttattattagatatcacaaattgtcaggtctatctttatttgaaggtagaggtagcctcaagcactttagttgggtttgttaaacaagcaagcaaagcggaaactacagctaagcatcttctgaatgagatcatcatcactatagaagaacctatgtcaaagatcttcaactcaagaaggaacagtgaggattagttcctttattgtcagcgtcagaactgtggcttggccagcctcttctcttaggtaaggcatgagcaccctaggcttcttctgtgtatctcttgctgcttaaatgtgtctccattaggggtgtatatccttttcgaagtcttctatattgaagaaaagccaacagcacaaaaagaccaaccaaagccaccagtgttcccatgactactaagagagttgtgggccaacctggagtttcttcaactgaaactggcagatcgatggcatagctgtagccaagttggtctgaggttaaaaagagttcttcattagtcactggagggaagaaaggaaccatgttgtacatccgattgtgaccaataggggccagctcctgaggccaggcatctgcaggaggattaaatcttttcatccactcatcaaagatggcatcagtaaaggaatgaagaaccacaaaaatgggatcattggcggctgaatgtggcaaagcgtttgtcccgttcaggaaggaatgaaccaaattatgaaggctcatcacttgagaatccagagtcccatctgctttatcaaacccttccaaagcattcctgaaactgaaggtagagttctggaagaagggaggattgtcaaacttctggagagacaggcaatctcgtatgtcttttaaggttggcaatttcatgctgtttcttcccatttgatttcttctcagcaaaccttcataggttccattgcacaaggtgaccaggtggttgtagtcatccaagctatcacagacagtttcccagctggagaatcttgagttccgactaatcagagtcggatcgtctggtctcgctgccccaaacagctggtctgtacacacatcacactcgttcctcccagtggcaaagttccagtagggcaaagcaaaagactcattgccaatgagtcgctggagatctctttccagacacaacaaatggtaccggtgccaggtaacaaatgcaggtccttgatgtgagaaatctatggccctgtaggggcgtcctggtcctaataatgtatctctaacagaataataatggagccacacaaaaaaatcataaacactgcagttggcaaactgcggctgggttccattgggcccaagcaggcccagccagtgttgtgtggtgatcacgtagtcggggtgtactctcttcttcgcgagatctaaggcgcccaagaactgctctctttcctgaggactcaaggaatggatgttctgccgaatcactggtggtttcttccgctcgcagttgggaccggtccagccaaacttgcagtctccacaattatagccggcaaagtttcctgtgcacttgcaggtccggtggaagaattttcttggccacagctcacggtcatcctggtttcgtaggatgtagggaccactccagggccttgtgtcggctcgcacctctgtgcactgcccccggccttgctgagagccacagacattggccgactctgcacccaggcgtgggcagcactccttgttcactaggctgtccaccgtcatgcagactcgggggaactgaccctgggctcctggcaggattttgcagcccaagcaactgagcagaaacccccaccaaagggggctcatggctttataattgggagagctctctctctctcttactttccttgtctctgtcgtacttttctccttatcttctactctttcagtcttttcttttcagtattttttatttttctttgctttctattcctttcttcttaaaaaaatacccacaagaatcacagaggttacatgtgtgcacggttacatgtgtgcacatgtgtacatgaacgtgcacacacaattttatgtgattcaaacaactaacagacttaatttccttagaagcgcctctaacaaccaaatttaatgagggtagcgcttctcaccatcttcccccgttaagtcaggctttgtctaattgagttaatttacagagcacccagtcatactacttattatgctggtatttctaaaccctctccctccctccttagctcttgactttaatctcgtgccgaattcggcacgagaattgttaaaacagaaatcagaggagccatcggtgtcaatacccttcctacaaactgcattattaagaagttcagggagtcttgggcacagaccaagccaggagatggataaaatgttaaaaaatcaagcaacttctgctacttctgaaaaggataatgatgatgaccaaagtgacaagggtacttataccattgagttagagaatcccaacagtgaggaagtggaagcaagaaaaatgattgacaaggtgtttggagtagatgacaatcaggattataataggcctgttatcaacgaaaaacataaagatctaataaaagattgggctctcagttctgctgcagcagtaatggaagaaagaaaaccactgactacatctggatttcaccactcagaggaaggcacatcttcatctggaagcaaacgttaggtttcacagtgggctagtttggctgccaatcatacaaggcatgatcaagaagaaaggataatggaattttctgcacctcttcctttagagaatgagacagagatcagtgagtctggcatgacagtgagaagtactggctctgcaacttccttggctagccagggagagagaaggagacgaactcttccccagcttccaaatgaagaaaagtctcttgagagccacagagcaaaggttgtaacacagaggtcagagataggagaaaaacaagacacagaacttcaggagaaagaaacacctacacaggtataccagaaagataaacaagatgctgacagacccttgagtaaaatgaacagggcagtaaatggagagactctcaaaactggtggagataataaaaccctacttcacttaggcagctctgctcctggaaaagagaaaagtgaaactgataaggaaacttctttggtaaagcaaacattagcaaaacttcaacaacaagaacaaagggaggaggctcagtggacacctactaaattgtcttccaaaaatgtttcaggtcagacagataaatgtagggaggaaacttttaaacaagaatcacaacctccagaaaaaaattcaggacattctacaagcaaaggagacagagtggcacaaagtgagagcaagagaagaaaagctgaggaaattctgaaaagtcagactccaaagggaggagacaagaaggaatcctccaagtcattagtgcgacaagggagcttcactatagaaaaacccagcccaaacatacccatagaacttattccccatataaataaacagacttcctctactccttcttctttagcattaacatctgcaagtagaatacgag

SEQ ID NO: 17 - DNA, 1737 bp, homo sapiens
atgacagggtccagaaactggcgagccacgagggacatgtgtaggtatcggcacaactatccggatctggtggaacgagactgcaatggggacacgccaaacctgagtttctacagaaatgagatccgcttcctgcccaacggctgtttcattgaggacattcttcagaactggacggacaactatgacctccttgaggacaatcactcctacatccagtggctgtttcctctgcgagaaccaggagtgaactggcatgccaagcccctcacgctcagggaggtcgaggtgtttaaaagctcccaggagatccaggagcggcttgtccgggcctacgagctcatgctgggcttctacgggatccggctggaggaccgaggcacgggcacggtgggccgagcacagaactaccagaagcgcttccagaacctgaactggcgcagccacaacaacctccgcatcacacgcatcctcaagtcgctgggtgagctgggcctcgagcacttccaggcgccgctggtccgcttcttcctggaggagacgctggtgcggcgggagctgccgggggtgcggcagagtgccctggactacttcatgttcgccgtgcgctgccgacaccagcgccgccagctggtgcacttcgcctgggagcacttccggccccgctgcaagttcgtctgggggccccaagacaagctgcggaggttcaagcccagctctctgccccatccgctcgagggctccaggaaggtggaggaggaaggaagccccggggaccccgaccacgaggccagcacccagggtcggacctgtgggccagagcatagcaagggtgggggcagggtggacgaggggccccagccacggagcgtggagccccaggatgcgggacccctggagaggagccagggggatgaggcagggggccacggggaagataggccggagcccttaagccccaaagagagcaagaagaggaagctggagctgagccggcgggagcagccgcccacagagccaggccctcagagtgcctcagaggtggagaagatcgctctgaatttggaggggtgtgccctcagccagggcagcctcaggacggggacccaggaagtgggcggtcaggaccctggggaggcagtgcagccctgccgccaacccctgggagccagggtggccgacaaggtgaggaagcggaggaaggtggatgagggtgctggggacagtgctgcggtggccagtggtggtgcccagaccttggcccttgccgggtcccctgccccatcggggcaccccaaggctggacacagtgagaacggggttgaggaggacacagaaggtcgaacggggcccaaagaaggtacccctgggagcccatcggagaccccaggcccccgcccagcaggacctgcaggggacgagccagccgagagcccatcggagaccccaggccccagcccggcaggacctacaagggatgagccagccgagagcccatcggagaccccaggcccccgcccggcaggacctgcaggggacgagccagccgagagcccatcggagaccccaggcccccgcccggcaggacctgcaggggacgagccagccgagagcccatcggagaccccaggccccagcccggcaggacctacaagggatgagccagccaaggcgggggaggcagcagagttgcaggacgcagaggtggagtcttctgccaagtctgggaagccttaa

SEQ ID NO: 18 - protein, 578 aa, homo sapiens
MTGSRNWRATRDMCRYRHNYPDLVERDCNGDTPNLSFYRNEIRFLPNGCFIEDILQNWTDNYDLLEDNHSYIQWLFPLREPGVNWHAKPLTLREVEVFKSSQEIQERLVRAYELMLGFYGIRLEDRGTGTVGRAQNYQKRFQNLNWRSHNNLRITRILKSLGELGLEHFQAPLVRFFLEETLVRRELPGVRQSALDYFMFAVRCRHQRRQLVHFAWEHFRPRCKFVWGPQDKLRRFKPSSLPHPLEGSRKVEEEGSPGDPDHEASTQGRTCGPEHSKGGGRVDEGPQPRSVEPQDAGPLERSQGDEAGGHGEDRPEPLSPKESKKRKLELSRREQPPTEPGPQSASEVEKIALNLEGCALSQGSLRTGTQEVGGQDPGEAVQPCRQPLGARVADKVRKRRKVDEGAGDSAAVASGGAQTLALAGSPAPSGHPKAGHSENGVEEDTEGRTGPKEGTPGSPSETPGPRPAGPAGDEPAESPSETPGPSPAGPTRDEPAESPSETPGPRPAGPAGDEPAESPSETPGPRPAGPAGDEPAESPSETPGPSPAGPTRDEPAKAGEAAELQDAEVESSAKSGKP

SEQ ID NO: 19 - protein, 176 aa, homo sapiens
MRVLGTVLRWPVVVPRPWPLPGPLPHRGTPRLDTVRTGLRRTQKVERGPKKVPLGAHRRPQAPAQQDLQGTSQPRAHRRPQAPARQDLQGMSQPRAHRRPQAPARQDLQGTSQPRAHRRPQAPARQDLQGTSQPRAHRRPQAPARQDLQGMSQPRRGRQQSCRTQRWSLLPSLGSL

SEQ ID NO: 20 - protein, 49 aa, homo sapiens
SPSETPGPRPAGPAGDEPAESPSETPGPRPAGPAGDEPAKTPSETPGPS

SEQ ID NO: 21 - protein, 50 aa, homo sapiens
AHRRPQAPAQQDLQGTSQPRAHRRPQAPAQQDLQGTSQPRAHRRPQAPAQ

SEQ ID NO: 22 - protein, 9 aa, homo sapiens
SLGSPVLGL

SEQ ID NO: 23 - protein, 10 aa, homo sapiens
RLASFYDWPL

SEQ ID NO: 24 - protein, 20 aa, homo sapiens
  VARIANT (2)...(17): Xaa at 2 is Thr or Met; Xaa at 4 is Gln or Arg; Xaa at 7 is Ala or Pro; Xaa at 16 is Arg or Gln.
  VARIANT 2, 4, 7, 16: Xaa = Any Amino Acid
GXSXPRXHRRPQAPAXQDLQ

SEQ ID NO: 25 - protein, 14 aa, homo sapiens
AHRRPQAPAQQDLQ

SEQ ID NO: 26 - protein, 20 aa, homo sapiens
GTSQPRAHRRPQAPARQDLQ

SEQ ID NO: 27 - protein, 20 aa, homo sapiens
GMSQPRAHRRPQAPARQDLQ

SEQ ID NO: 28 - protein, 20 aa, homo sapiens
GTSQPRAHRRPQAPAQQDLQ

SEQ ID NO: 29 - protein, 20 aa, homo sapiens
GTSQPRPHRRPQAPARQDLQ

SEQ ID NO: 30 - protein, 20 aa, homo sapiens
  VARIANT (1)...(20): Xaa at 9 is Arg or Ser; Xaa at 14 is Ala or Thr; Xaa at 15 is Gly or Arg; Xaa at 20 is Glu or Lys
  VARIANT 9, 14, 15, 20: Xaa = Any Amino Acid
SPSETPGPXPAGPXXDEPAX

SEQ ID NO: 31 - protein, 20 aa, homo sapiens
SPSETPGPRPAGPAGDEPAE

SEQ ID NO: 32 - protein, 20 aa, homo sapiens
SPSETPGPSPAGPTRDEPAE

SEQ ID NO: 33 - protein, 20 aa, homo sapiens
SPSETPGPSPAGPTRDEPAK

SEQ ID NO: 34 - DNA, 6670 bp, homo sapiens
ccggcccggtcgacccgccgccgccgagcatggacgaccccgactgcgactccacctgggaggaggacgaggaggatgcggaggacgcggaggacgaggactgcgaggaggccgaggccgccggcgcgagggacgcggacgcaggggacgaggacgaggagtcggaggagccgcgggggccgtgcccagctcgttccagtccagaatgacagggtccagaaactggcgagccacgagggacatgtgtaggtatcggcacaactatccggtacgtacctgcccctgccccgggacacagaaccctcccgccagctgctcttctcaggcagaatgtcccaggttctactggaaggctggcctggcttgctgtgccagggccacagttctgggcaggaccctgcctggggcacaacctggtatagattcagagccctgcccttcccctctcgcgggagaccgggggcatcccctacttttctgagcttcagcacaccgtcgccttgcaaacatggccatagtgccagcctcgtgatgcacacgtgtggggtccgtgataccgcccacacagcaccccacccccatgccagccctgtctcctgcaggttgtcctcagccattaccctccacacccctgaatcacggaaacccctgtgctgccttcagggtgctgaggaggggaccctaggcctggcctggctggcgaaatggggaagggggtccctggggcttggaggcagctgctgtcctctgaatggcccccacctgcagagtgaggagccaggcgggctcttggggtattgggccagcctggaggtttgcagatgcgcctccccgaaagacacggagggccccagggcagcttgtgtctgatggatccctgctgtcccctttctctggcccttcaggatctggtggaacgagactgcaatggggacacgccaaacctgagtttctacagaaatgagatccgcttcctgcccaacggtaggtgcctcacaaccactggcagccggcgcttccatccttgcctggcaggggtggtccactgggccctggtttgggatgtacccggcgattaccagggccacggctttcaaatagccccacagtgccccctggaggcggccagagggcatccaggatccccggcttggaatctccgcagtggtgtttgggtcggggtgggctatctctgtaaagagccagagactgaatatctgcagcttttcaggccacacagtctctatcaaaactactgaactcctgcacgacctcatgaaagccacatgtaaaccaacaggctaaggatagcttttatatttttaaatggttgcaaaaaattaaaagtagaggccgggcctggtggctcacgcttgtaatcccagcactttgggaggccaaggcgggtggatcacaaagtcatgagttcgagaccagcctggccaatatggtgaaaccctgtctctactaaaaatacaaaaaaaattagccaggtgtggtagcgtgcgcctgtagtcccagctactcaggaggctgaggcaggagaatcgcttgaacttgggaggtggaggttgcgtgagccgagatctcgccactgccctccagcctgggcaacagaatgagactctgtctcaaaaaaaaaaaaaaaaaaaaaaagaacagtgttctattactttgatattataagaaattaaaatttcagtgtcaataaagcttcacggcacgtgcccacacctgtttatgcacgtgccgggctgcaacgtgaaacagagactgggtggctgcacgaaacactgtcaggcccccatctcaggagactgtgcctgccacatcatgggactgggttcagctgggggctgtgtttgaggacagatttctgtggctgacgggccacgcttttgcaggaggcactgaggccccctcctgtacagacaggtcctggtcgaggaccagtggttcccccagagggacttccgggctctcagtggggtttgccgccagccagcaacaagggtccaggtcacagatggcagcagggcaagggctgtgcatcggaggagggatgggcagaaccactgagcctagcaccctgaagagcctcaggagatgagggcgaggaatagcttggggaagctgctggcagcactctctccttatgtcccccacccacaccccgtcctcctgagcacgggcagggtggctgccacagctgacttgtcccatggggctcccaggctgtttcattgaggacattcttcagaactggacagacaactatgacctccttgaggacaatcactcctacatccagtggtgagttggggaggatgggaggatggggggttggcgaggccacagggggagggccctcccaggcaggagccctcccgacgctgcttcctggcacggactgagcactccctgcccttcctggaagctggtgtccacattacacggatggacattactggacatgcaagggccgctgagggaaggctcaggtctgtccccaatctgtgggctcttggtgaccagggagacaggaagggctgcacacaagctccctgaaacaggagagggggcaggcactgctcgagaggggcagccttagtgccaggaggcctcagtgagcctcaaggggcggcccccgtctcggactcagctggggcagggtcctggcgtagtgctgggatgagacttgtggcaggcgtcttggggacccacaatgtccccggaaacatagcatgccctgctttggggacagctcgctgggcgtccacagggagcctctcgtggtgtgtgacgtgtcatgagtgacaacacaagagagggaagagtgaggagtagaaaggaaggccctgtccgcccgggctggatggctgcagggcccctcgctgcctgagtcacctccccagtcctgctgagcatgcaggctgtgacttacgcaccacacacctcctgtcctgggcccttgcacgctcacctgagctctcccaccctcgctcctccacctagccactccctcctagagttgagcctgggaacccagaggggattggaactgccccgggctacacagcgagcacctgcccgaggtctggaagcggctctcctaatcccttgcctgagcatctcttctcctgcaggctgtttcctctgcgagaaccaggagtgaactggcatgccaagcccctcacgctcagggaggtcgaggtgagccaggccttggggctgtgactggaggggaagatggggaggcctgggcaagccacgcgcagagacggggtcgcctctggcagtaggcatttggtgccccctcagggtcccttggaaggcagaagggccgaaagagcctccagtatgatgaccttccctccccagtggtcctcaccccaccaggcatctagagaaactcaatttccgaggctgtttgtccccatgcgggagtctggggctgagcctgagagacccggaatggcgggacctgcctttctcaccactggtttaggctggagtggggtcacccaagtgtggaagctgcccctgagggatgcagggacttagagagccgaggggcccctcctggcactttcccatggccagcgacttcctctcaggggagggcggccatgcaggccctggtgagagctcggagcagctcagaggagagatggaggcatgagtgtgtctcttgtcactttaccctaaaagtctgagtccaggctaggtgccgtggctcacgtttgtaatcccagcactttgagaggctgaggcgggtggatcacctgaggtcaggagtttgagaccagctggccaacgtgacaaaaccccgtttctacttaaaaatagaaaaaaattagccaggtgtggtggcaggcaactgtaatcccagctactcgggaggcagaggttgcagtgacttgaggattgcagtgacccttgaggctacaccattgcactccagcctgggcaacagagcaagactctgtctctaaataaatgtctgtctgtctgtctgtctagtccccaggcctctgagtgagctgggacccacggcctcccccacactcaggaaccggggcttcagggatggtgcctgagctctccaagggggtcttttccaggtgtttaaaagctcccaggagatccaggagcggcttgtccgggcctacgcagctcatgctgggcttctacgggatccggctggaggaccgaggcacgggcacggtgggccgagcacagaactaccagaagcgcttccagaacctgaactggtgaggcccggctgctcccgcccacccccaccccggcgcagaacagggccacgtcaggtttcgggcaggtcacagagcgctgctgcagacggagaactccagggctgtttgggcaatcgaccaaggccctgagtcccctccttgctgcctgtagagccgggcggtccctccccgataggttggtgagaaggtaaagaatgctcttcctgcacccaggaggtggcagtccacatagagaagcaggcaggggtgatggagaacctgcaggcggtacagccagttctcacagggtgttcgaggcgcctgtaccctgcggggccccttgtggctctcatccagccagctcctccccaccagggggactcggggtacagccaccctctaggtgatgcgtcagcatccccctaacccccgtgtgtgtaggcccacaggccattttcacagggatggcatgagtcctcccctgggcccagagaggtaaatggggagagagactgaatcgtgggccagggtggggagacctcgtggagccgggtgggagggcaggccagggcggggtgcggcccagcaggaggggccccggccattgacctctcctgacccggatctctcgcagcgcagccacaacaacctccgcatcacacgcatcctcaagtcgctgggtgagctgggcctcgagcacttccaggcgccgctggtccgcttcttcctggaggagagcctggtgcggcgggagctgccgggggtgcggcagagtgccctggactacttcatgttcgccgtcggctgccgacaccagcgccgccagctggtgcacttcgcctgggagcacttccggccccgctgcaagttcgtctgggggccccaagacaagctgcggaggttcaagcccagctctctgccgcatccgctcgagggctccaggaaggtggaggaggaaggaagccccggggaccccgaccacgaggccagcacccagggtcggacctgtgggccagagcatagcaagggtgggggcagggtggacgaggggccccagccacggagcgtggagccccaggatgcgggacccctggagaggagccagggggatgaggcagggggccacggggaagataggccggagcccttaagccccaaagagagcaagaagaggaagctggagctgagccggcgggagcagccgcccacagagccaggccctcagagtgcctcagaggtggagaagatcgctctgaatttggaggggtgtgccctcagccagggcagcctcaggacggggacccaggaagtgggcggtcaggaccctggggaggcagtgcagccctgccgccaacccctgggagccagggtggccgacaaggtgaggaagcggaggaaggtggatgagggtgctggggacagtgctgcggtggccagtggtggtgcccagaccttggcccttgccgggtcccctgccccatcggggcaccccaaggctggacacagtgagaacggggttgaggaggacacagaaggtcgaacggggcccaaagaaggtacccctgggagcccatcggagaccccaggccccagcccagcaggacctgcaggggacgagccggccgagagcccatcggagaccccaggcccccgcccagcaggacctgcaggggacgagccagccgagagcccatcggagaccccaggcccccgcccggcaggacctgcaggggacgagccagccaagaccccatcggagaccccaggccccagcccggcaggacctacaagggatgagccagccgagagcccatcggagaccccaggcccccgcccggcaggacctgcaggggacgagccagccgagagcccatcggagaccccaggcccccgcccggcaggacctgcaggggacgagccagccgagagcccatcggagaccccaggccccagcccggcaggacctacaagggatgagccagccaaggcgggggaggcagcagagttgcaggacgcagaggtggagtcttctgccaagtctgggaagccttaaggaaaggagtgcccgtcggcgtcttggtcctcctgtccctgctgcaggggctggggcctccggagctgctgcgggctcccctcaggctctgcttcgtgacccgtgacccatgacccacagtgctggcctcctgtggggccactatagcagccaccagaagccgcgaggccctcagggaagcccaaggcctgcagaagcctcctggcctggctgtgtcttccccacccagctctcccctgcgcccctgtctttgtaaattgacccttctggagtggggggcggcgggcagggctgcttttcttagtctgatgccaagcaaggccttttctgaataaattcatttgactttgaaaaaaaaaaa

SEQ ID NO: 35 - protein, 618 aa, homo sapiens
MTGSRNWRATRDMCRYRHNYPDLVERDCNGDTPNLSFYRNEIRFLPNGCFIEDILQNWTDNYDLLEDNHSYIQWLFPLREPGVNWHAKPLTLREVEVFKSSQEIQERLVRAYAAHAGLLRDPAGGPRHGHGGPSTELPEALPEPELRSHNNLRITRILKSLGELGLEHFQAPLVRFFLEESLVRRELPGVRQSALDYFMFAVGCRHQRRQLVHFAWEHFRPRCKFVWGPQDKLRRFKPSSLPHPLEGSRKVEEEGSPGDPDHEASTQGRTCGPEHSKGGGRVDEGPQPRSVEPQDAGPLERSQGDEAGGHGEDRPEPLSPKESKKRKLELSRREQPPTEPGPQSASEVEKIALNLEGCALSQGSLRTGTQEVGGQDPGEAVQPCRQPLGARVADKVRKRRKVDEGAGDSAAVASGGAQTLALAGSPAPSGHPKAGHSENGVEEDTEGRTGPKEGTPGSPSETPGPSPAGPAGDEPAESPSETPGPRPAGPAGDEPAESPSETPGPRPAGPAGDEPAKTPSETPGPSPAGPTRDEPAESPSETPGPRPAGPAGDEPAESPSETPGPRPAGPAGDEPAESPSETPGPSPAGPTRDEPAKAGEAAELQDAEVESSAKSGKP

SEQ ID NO: 36 - DNA, 355 bp, homo sapiens
caaagtcaaatgaatttattcagaaaaggccttgcttggtatcagactaagaaaagcagccctgcccgccgccccccactccagaagggtcaatttacaaagacaggggcgcaggggagagctgggtggggaagacacagccaggccaggaggcttctgcaggccttggctatccctgagggcctcgcggcttctggtggctgctatagtggccccacaggaggcacgcactgtgggtcatgggtcacgggtcacgaagcagagcctgaggggagcccgcagcagctccggagccccaggccctgcagcagggacaggaggaccaagacgccgacggcactcctttccttaaggc

SEQ ID NO: 37 - DNA, 270 bp, homo sapiens
gaagggtcaatttacaaagacaggggcgcaggggagagctgggtggggaagacacagccaggccaggaggcttctgcaggccttgggttccctgagggcctcgcggcttctggtggctgctatagtggccccacaggaggcagcactgtgggtcatgggtcacgggtcacgaagcagagcctgaggggagcccgcagcagctccggagccccagccctgcagcagggacaggaggaccaagacgccgacgggactcctttccttaaggct

SEQ ID NO: 38 - DNA, 141 bp, homo sapiens
aaagtcaaatgaatttattcagaaaaggccttgcttggtatcagactaagaaaagcagccctgcccgccgccccccactccagaagggtcaatttacaaagacaggggcgcaggggagagctgggtggggaagacacagcc

SEQ ID NO: 39 - DNA, 192 bp, homo sapiens
caaggcgggggaggcagcagagttgcaggacgcagaggtggagtcttctgccaagtctgggaagccttaaggaaaggagtgcccgtcggcgtcttggtcctcctgtccctgctgcaggggctggggctccggagctgctgcgggctccctcaggctctgcttcgtgacccgtgacccatgacccacagtgct

SEQ ID NO: 40 - DNA, 309 bp, homo sapiens
  misc_feature 1, 80, 254, 265, 275, 282, 290, 304: n = A,T,C or G
ncaaagtcaaatgaatttattcagaaaaggccttgcttggtatcagactaagaaaagcagccctgcccgccgccccccantccagaagggtcaatttacaaagacaggggcgcaggggagagctgggtggggaagacacagccaggccaggagcttctgcaggccttgggcttccctgagggcctcgcggcttctgggtggctgctatagtggccccacaggaggccatgcactgtgggggtcattgggtcacngggtcacgaangcatagcctnagggggnagcccgtnagcagctccggganggccc

SEQ ID NO: 41 - DNA, 178 bp, homo sapiens
gggaagcccaaggcctgcagaagcctccgtggcctggcatgtgtcttccccacccagctctcccctgcgcccctgtctttgtaaattgacccttctggagtggggggcggcgggcagggctgcttttcttagtctgataccaagcaaggccttttctgaataaattcatttgactttg

SEQ ID NO: 42 - DNA, 166 bp, homo sapiens
  misc_feature 15, 22, 24, 76, 77, 119, 153, 163: n = A,T,C or G
cggcctgcagaagcntcctggncntggttgttttttccccacccagctctcccctgcgcccctttttttttaaatnnacccttctggagtggggggcggcgggcagggctgcttttttnagtctgatgccaagcaaggccttttttgaataanttcatttganttt

SEQ ID NO: 43 - DNA, 209 bp, homo sapiens
  misc_feature 11, 90, 138, 166, 185, 190, 200: n = A,T,C or G
gaaggtggatnagggtgctgtggacagtgctacggtggccagtggtggtgcccagaccttggcccttgccgggtcccctgccccatcgcncggccaaggctggacacagtgagaacggggttgaggaggacacagaangtcaaacggggcccaaagaaggtacccntggggagcccatcagagancccangccccagccnggcagggac

SEQ ID NO: 44 - DNA, 241 bp, homo sapiens
ttttttttttttttttttttttttcaaagtcaaatgaatttattcagaaaaggccttgcttggcatcagactaagaaaagcagccctgcccgccgccccccactccagaagggtcaatttacaaagacaggggcgcttgggagagctgggtggggaagacacagccaggccaggaggcttctgcaggccttgggcttccctgagggcctcgcggcttctggtggctgctatagtggcccca

SEQ ID NO: 45 - DNA, 5922 bp, homo sapiens
gcggccgcggggaccctcggcgtggtcctctgaccctgcaaacccgcgacggaggaaggggaggtcctgcccgaggcgccagcccgaggaggaggatgcccatttaacccgccctcgcctgccgggcgcttgcctcggtgcccgccgccggagcctccgagccgcgcccgtggaagtgctgcatggggcagggctgctgaagcgcggagttcggggtcgcgccgctcccaggcaggcgcgggagcccggtgcggcagttggcacagtttcggcggcgccttctgcgcgggagtggggggcgcggtgcgcccggccggcctccgcggtgccctggtgaggcgagagttatggagccgcccagctgcattcaggatgagccgttcccgcaccccctggagcccgagccgggcgtctcagctcagcccggccccgggaagccaagcgataagcggttccggctgtggtacgttggggggtcgtgcctggaccacaggaccacgctgcctatgctgccctggctcatggccgagatccgcaggcgcagccagaagcccgaggcgggcggctgcggggcgccggcggcccgagaggtgatcctggtgctcagcgcgcccttcctgcgttgcgtccccgcgccgggcgctggggcctcggggggcactagtccgtcggccacgcagcccaacccggcggtattcatcttcgagcacaaggcgcagcatatctcgcgcttcatccacaacagccacgacctcacctactttgcctacctgatcaaggcgcagcccgacgaccccgagtcgcagatggcctgccacgttttccgcgccacagaccccagccaggttcctgatgttattagcagcataaggcaattatctaaagcggccatgaaagaggatgccaaacccagcaaagataatgaggacgccttttacaactctcagaagttcgaagtcctgtactgtggaaaggtgaccgtgacccacaagaaggccccctcaagcctcatcgatgactgcatggagaagttcagcctgcacgaacagcagcgcctgaagatccaaggcgagcagcgcggtccggacccaggagaggacctggctgacttggaggtggtggtgcccgggtcccccggagactgcctgccggaggaggctgacggcaccgacacccaccttggcttacctgccggggccagccagcctgccctgaccagctctcgggtctgcttccctgagcggattttggaagattctggctttgatgagcagcaggagtttcggtctcggtgcagcagtgtcaccggcgtgcaacggagagttcacgagggcagccagaaatcccagccgcgacggagacacgcgagcgcacccagtcacgtccagccctcggactcggagaagaacaggaccatgctcttccaggttgggcgatttgagattaaccttatcagtccagacactaaatcagttgtgctagaaaagaattttaaagatatctcctcttgttctcagggtataaagcatgtggatcactttggctttatctgccgggagtctccagagcctggacttagccagtatatttgttatgtattccagtgtgccagcgaatctctggttgatgaggtaatgctgactctgaaacaggccttcagtacggcggctgccctgcagagtgccaagacgcagattaaactgtgtgaggcctgcccgatgcactctttgcataagctctgtgaaaggattgaaggtctctacccaccaagagccaagctggtgatacagaggcatctctcatcactgacagataatgagcaagctgacatctttgaaagagttcagaaaatgaagccagtcagtgaccaggaagaaaatgaacttgtgattttacacctgaggcagctgtgtgaagccaagcagaagacacacgtgcacatcggggaaggcccttctactatttcaaatagtacaatcccagaaaatgcaacaagcagtggaaggttcaaacttgacattctgaaaaataaagctaagagatccttaactagctccctggaaaatatcttctcaaggggagctaacagaatgagaggtcggcttggaagtgtggacagttttgaacggtccaacagtcttgcttcagagaaggactactcaccaggggattctccaccagggacaccgccagcgtccccaccgtcctcagcttggcaaacgtttcccgaagaggattccgactccccgcagtttcgaagacgggcacacacgttcagccacccaccttcaagcacaaagagaaagctgaatttgcaggatgggagggctcagggtgtgcgttcccctctgctgaggcagagctccagtgaacagtgcagcaatctttcgtcagttcgacgcatgtacaaggagagtaattcttcctccagtcttccaagtcttcacacttccttctctgccccttccttcactgccccctctttcctgaaaagcttttaccagaattcaggtagactgtccccacagtatgaaaatgaaatcagacaagacactgcttcagaatcaagtgatggagaagggagaaaaaggacctcatctacctgcagcaatgagtccctaagtgtgggaggaacctctgtcactcctcgccggatctcctggcggcagcgcattttcctcagggttgcttctcccatgaacaaatctccctcagcaatgcaacagcaagatggattggacaggaacgagctgctgccactgtcccccctctctccaaccatggaggaggaaccgctggttatattcctgtctggggaggatgacccagaaaagattgaagaaagaaagaaatcaaaagaactgaggagcttgtggagaaaagctatacaccaacaaatcttgttacttcgaatggaaaaagaaaaccagaaacttgaaggagcaagcagagatgaactccagtccagaaaagttaaattagactatgaagaagttggtgcatgtcagaaagaggtcttaataacttgggataagaagttgttaaactgcagagctaaaatcagatgtgatatggaagatattcatactcttcttaaagaaggagttcccaaaagtcgacgaggagaaatttggcagtttctggctttacagtaccgactcagacacagattgcctaataaacaacagcctcctgacatatcctataaggaacttttgaagcagctcactgctcagcagcatgcgattctcgtggatttaggaaggacgtttcctactcacccttacttttcagtacagcttgggccaggacagctgtcactgtttaacctcctgaaagcctattctttgctggacaaagaagtgggatactgtcaggggatcagctttgtggctggagtcctgcttctgcacatgagtgaagagcaagcctttgaaatgctgaaattcctcatgtatgacctcggcttccgcaagcagtacagacctgacatgatgtcgctgcagattcaaatgtaccagctgtccaggctccttcatgactatcacagagatctctacaatcaccttgaagaaaatgaaatcagccccagtctttatgctgccccctggttcctcacattgtttgcctctcagttttcattaggatttgtagccagagtttttgatattatttttcttcagggaactgaagttatattcaaggttgcactcagcctactgagcagccaagagacacttataatggaatgtgagagctttgaaaatattgttgagtttcttaaaaacacgctacctgatatgaatacctctgaaatggaaaaaattattacccaggtttttgagatggatatttctaagcagttgcatgcctatgaggtggaatatcatgtgctacaggatgagcttcaggaatcttcatattcctgtgaggatagtgaaactttggagaagctggagagggccaatagccaactgaaaagacaaaacatggacctcctagaaaaattacaggtagctcatactaaaatccaggccttggaatcaaacctggaaaatcttttgacgagagagaccaaaatgaagtctttaatccggaccctggaacaagaaaaaatggcttatcaaaagacagtggagcaactccggaagctgctgcccgcggatgctctagccaattgtgacctgttgctgagagacctaaactgcaaccctaacaacaaagccaagataggaaataagccataattgaagaggcacggcctcagcagaaagtgctccttagaatactacagagaggaagagcctgcatgtcgctggcccaaggctggaccctgaagctgatggaaccacctaatactggtgctgagctcctagtcacagcaggtggacctcgtgctcatcagagcatgccaatcctaagccattggacatatgtagactggtttttgttgttgctatgtacatataaatatatatataaaatgaacatagttcatgctttcagataaaatgagtagatgtatatttagattaatttttttagtcagaacttcatgaaatccacaccaaaggaaaggtaaactgaaatttcccttggacatatgtgaaatctttttgtctttatagtgaaacaaagccagagcatctttgtatattgcaatatacttgaaaaaaatgaatgtatttttttctccaaagaacagcatgtttcactcaatggtgaaaaggtggaaacatttatgtaactttatgtgtatctgtcttgatatctactgacattgtctatatgaggaaaatgattactggtcatgctcctgtgagttttttgggaaggtagggtcatttctccctgcctgctttgtgccaactagcatgttgcatctacatgcattatgagtctggttaggcattactttaaacatacataaagagacagtaggacattgtggctgagtctacccagctcaaggtaaaggagaatgttgctaattttttagcaaactagaccagcattattactcaaactaaaaatatcacacctgaaaaatttaatttaggacctaaaatgtctagattagctttctgctttttttatttgaataactcattcagttgtgaatgaattcctctttatttggtgccacagtcaccaaatgacaaggatttgccactttcccaccaaattgtgagtgcttgtaatttaggtctctctaccttaaattcagtataaggaaacgtaattatgattgattttttccaaagatgacaagctgtgttgaaatacattttttcttttgaccaattgacagaatctaataagctttaataatcttccccttttatgtgaaaagttttgagaactgtgaaatgtttaggaacaaactgttgaaatccattggaagggaaaaaagaaagtggtaccagtgttaccagctcaactaaaacctgcaattctgcatttcaactcttcacttcctcagcctacaaatagctcattagatgacattcacgcatgctgggtataggcaaggaaagtaattttcaaagtacatttgcagttctctttttcagagatgattctatgatagtgcctctgaaagttgatgcagcatttttgcctttccaaaaagtatttatcctcactgctttttgcagtacttgtattttcacagatggattatctggggtaattttcttcaaagggagtttgttatacacagtgaaaatgtattatagagtagaatagtaaagctctaggggtttcagaaagctttgatgaacagatgacaaacatctgaaaccccctccgcactgttacccagtgtgtatataatgacttgttatagctcagtgtgcccttgaatccatacagtttcttaaaagacaataaaatcttattaataaagttaatgtaacttct

SEQ ID NO: 46 - protein, 1299 aa, homo sapiens
MEPPSCIQDEPFPHPLEPEPGVSAQPGPGKPSDKRFRLWYVGGSCLDHRTTLPMLPWLMAEIRRRSQKPEAGGCGAPAAREVILVLSAPFLRCVPAPGAGASGGTSPSATQPNPAVFIFEHKAQHISRFIHNSHDLTYFAYLIKAQPDDPESQMACHVFRATDPSQVPDVISSIRQLSKAAMKEDAKPSKDNEDAFYNSQKFEVLYCGKVTVTHKKAPSSLIDDCMEKFSLHEQQRLKIQGEQRGPDPGEDLADLEVVVPGSPGDCLPEEADGTDTHLGLPAGASQPALTSSRVCFPERILEDSGFDEQQEFRSRCSSVTGVQRRVHEGSQKSQPRRRHASAPSHVQPSDSEKNRTMLFQVGRFEINLISPDTKSVVLEKNFKDISSCSQGIKHVDHFGFICRESPEPGLSQYICYVFQCASESLVDEVMLTLKQAFSTAAALQSAKTQIKLCEACPMHSLHKLCERIEGLYPPRAKLVIQRHLSSLTDNEQADIFERVQKMKPVSDQEENELVILHLRQLCEAKQKTHVHIGEGPSTISNSTIPENATSSGRFKLDILKNKAKRSLTSSLENIFSRGANRMRGRLGSVDSFERSNSLASEKDYSPGDSPPGTPPASPPSSAWQTFPEEDSDSPQFRRRAHTFSHPPSSTKRKLNLQDGRAQGVRSPLLRQSSSEQCSNLSSVRRMYKESNSSSSLPSLHTSFSAPSFTAPSFLKSFYQNSGRLSPQYENEIRQDTASESSDGEGRKRTSSTCSNESLSVGGTSVTPRRISWRQRIFLRVASPMNKSPSAMQQQDGLDRNELLPLSPLSPTMEEEPLVIFLSGEDDPEKIEERKKSKELRSLWRKAIHQQILLLRMEKENQKLEGASRDELQSRKVKLDYEEVGACQKEVLITWDKKLLNCRAKIRCDMEDIHTLLKEGVPKSRRGEIWQFLALQYRLRHRLPNKQQPPDISYKELLKQLTAQQHAILVDLGRTFPTHPYFSVQLGPGQLSLFNLLKAYSLLDKEVGYCQGISFVAGVLLLHMSEEQAFEMLKFLMYDLGFRKQYRPDMMSLQIQMYQLSRLLHDYHRDLYNHLEENEISPSLYAAPWFLTLFASQFSLGFVARVFDIIFLQGTEVIFKVALSLLSSQETLIMECESFENIVEFLKNTLPDMNTSEMEKIITQVFEMDISKQLHAYEVEYHVLQDELQESSYSCEDSETLEKLERANSQLKRQNMDLLEKLQVAHTKIQALESNLENLLTRETKMKSLIRTLEQEKMAYQKTVEQLRKLLPADALANCDLLLRDLNCNPNNKAKIGNKP

SEQ ID NO: 47 - DNA, 2020 bp, homo sapiens
gttcgaggagctgctgctgctgaggcggcggcaactgcattgaggtggtggcggcgctgccggccccggccgctcgctctcggctcgccttccagcctcgcctgagcccgccgggcccgcgccggccagcgcctgccctatgagtgtgtcactggttgttatccgattggagctcgcggaacactcgcctgtccccgccggcttcggcttcagcgccgcggccggggaaatgtctgatgaggagataaaaaagacgacactagcctcagctgtagcctgtttagaaggcaagtcaccaggagagaaagtagcgattatccatcagcatctcggccgtcgagaaatgacagatgtgatcattgagaccatgaagtccaacccagatgaactaaaaactacagtggaagaaaggaagtcttcagaagcctcccccactgcgcaaagaagtaaagatcacagtaaggaatgcataaacgctgccccagattctccgtccaaacagcttccagaccagatttcattcttcagtggaaatccatcagttgaaatagttcatggtattatgcacctatataagacaaataagatgacctccttaaaagaagatgtgcggcgcagtgccatgctgtgtattctcacagtccctgctgcaatgaccagtcatgaccttatgaagtttgttgccccatttaacgacgtaattgaacaaatgaaaattatcagagactctactcccaaccaatatatggtgctgataaagtttcgtgcacaggctgatgcggatagtttttatatgacatgcaatggccgccagttcaactcaatagaagatgacgtttgccagctagtgtatgtggaaagagctgaagtgctcaaatctgaagatggcgccagcctcccagtgatggacctgactgaactccccaagtgcacggtgtgtctggagcgcatggacgagtctgtgaatggcatcctcacaacgttatgtaaccacagcttccacagccagtgtctacagcgctgggacgataccacgtgtcctgtttgccggtactgtcaaacgcccgagccagtagaagaaaataagtgttttgagtgtggtgttcaggaaaatctttggatttgtttaatatgcggccacataggatgtggacggtatgtcagtcgacatgcttataagcactttgaggaaacgcagcacacgtatgccatgcagcttaccaaccatcgagtctgggactatgctggagataactatgttcatcgactggttgcaagtaaaacagatggaaaaatagtacagtatgaatgtgagggggatacttgccaggaagagaaaatagatgccttacagttagagtattcatatttactaacaagccagctggaatctcagcgaatctactgggaaaacaagatagttcggatagagaaggacacagcagaggaaattaacaacatgaagaccaagtttaaagaaacaattgagaagtgtgataatctagagcacaaactaaatgatctcctaaaagaaaagcagtctgtggaaagaaagtgcactcagctaaacacaaaagtggccaaactcaccaacgagctcaaagaggagcaggaaatgaacaagtgtttgcgagccaaccaagtcctcctgcagaacaagctaaaagaggaggagagggtgctgaaggagacctgtgaccaaaaagatctgcagatcaccgagatccaggagcagctgcgtgacgtcatgttctacctggagacacagcagaagatcaaccatctgcctgccgagacccggcagaaatccaggagggacagatcaacatcgccatggcctcggcctcgagccctgcctcttcggggggcagtgggaagttgccctccaggaagggccgcagcaagaggggcaagtgaccttcagagcaacagacatccctgagactgttctccctgacactgtgagagtgtgctgggaccttcagctaaatgtgagggtgggccctaataagtacaagtg

SEQ ID NO: 48 - protein, 600 aa, homo sapiens
MSVSLVVIRLELAEHSPVPAGFGFSAAAGEMSDEEIKKTTLASAVACLEGKSPGEKVAIIHQHLGRREMTDVIIETMKSNPDELKTTVEERKSSEASPTAQRSKDHSKECINAAPDSPSKQLPDQISFFSGNPSVEIVHGIMHLYKTNKMTSLKEDVRRSAMLCILTVPAAMTSHDLMKFVAPFNDVIEQMKIIRDSTPNQYMVLIKFRAQADADSFYMTCNGRQFNSIEDDVCQLVYVERAEVLKSEDGASLPVMDLTELPKCTVCLERMDESVNGILTTLCNHSFHSQCLQRWDDTTCPVCRYCQTPEPVEENKCFECGVQENLWICLICGHIGCGRYVSRHAYKHFEETQHTYAMQLTNHRVWDYAGDNYVHRLVASKTDGKIVQYECEGDTCQEEKIDALQLEYSYLLTSQLESQRIYWENKIVRIEKDTAEEINNMKTKFKETIEKCDNLEHKLNDLLKEKQSVERKCTQLNTKVAKLTNELKEEQEMNKCLRANQVLLQNKLKEEERVLKETCDQKDLQITEIQEQLRDVMFYLETQQKINHLPAETRQKSRRDRSTSPWPRPRALPLRGAVGSCPPGRAAARGASDLQSNRHP

SEQ ID NO: 49 - DNA, 226 bp, homo sapiens
  misc_feature 163, 168: n = A,T,C or G
ctgggatactcccctcccagggtgtctggtggcaggcctgtgcctatccctgctgtccccagggtgggccccgggggtcaggagctccagaagggccagctgggcatattctgagattggccatcagcccccatttctgctgcaaacctggtcagagccagtnttccntccatgggacctaaagacagtgccaagtgcctgcaccgtggaccacagccgagccact

SEQ ID NO: 50 - DNA, 441 bp, homo sapiens
gaaaaaaaaaacgagtatctattaactggccactaacagttgcctttcttacattaatttatacactattttgttcagccagtgtttttaaaaaaaatctatgaaaagtgtacttccggttttctgtgattacttatctgggcttgatctgaccagtgaaatgacattgccctatttggacctctgaggttctatttagctttgcagatgtacatagtatcccagtgatctgcaaaattaatgccttttccaagaaaaaatcttttcttctctgtatcagttaattctgacagtgttagtgattctgtcttcattataggccttatttccattatctctttctttatagtattttttgttataaagaaaacagtctttctgtgtatacctacggatgagggtattatttaaactgccaacaatatccaagacatggtcaat

SEQ ID NO: 51 - DNA, 393 bp, homo sapiens
aagtctacaggtaagcagacatttctatacatgtcctggtcactctttctaaagtatttataattaggttattgaccatgtcttggatattgttggcagtttaaataataccctcatccgtaggtatacacagaaagactgttttctttataacaaaaaatactataaagaaagagataatggaaataaggcctataatgaagacagaatcactaacactgtcagaattaactgatacagagaagaaaagattttttcttggaaaaggcattaattttgcagatcactgggatactatgtacatctgcaaagctaaatagaacctcagaggtccaaatagggcaatgtcatttcactggtcagatcaagcccagataagtaatcacagaaaac

SEQ ID NO: 52 - DNA, 427 bp, homo sapiens
tttttttttgtctatcagtcaccttgaaactggtaatctgattcaagttaaacaatgttccttttgaatctagaaaacaagagaaatgcaaagtcattattccctcattctatgcttccatttactctaagaattcagaaacaaacatgtgggtaacttcctgttatcttaaaaaaagaatcatcccttcggtattcccttaactatctggaacttgtactgtcattttataatttaccatgtgacataattgtttgacctgcctcttttatttgatgcatgacttctcagagaacctgttatcaactcactgtgtaaaaccacgatgaaatgaaggataactgatcacaaagaattatgtcttttgatatccaacaaatttacaaattataagagaaaaatgcaattttttaaaaaaggatatcct

SEQ ID NO: 53 - DNA, 417 bp, homo sapiens
aaaaacactggctgaacaaaatagtgtataaattaatgtaagaaaggcaactgttagtggccagttaatagatactcgtttttttttttctcttcagttgcccactattattgcttatttttccttttcttgtctatcagtcaccttgaaactggtaatctgattcaagttaaacaatgttccttttgaatctagaaaacaagagaaatgcaaagtcattattccctcattctatgcttccatttactctaagaattcagaaacaaacatgtgggtaacttcctgttatcttaaaaaaagaatcatcccttcggtattcccttaactatctggaacttgtactgtcattttataatttaccatgtgacataattgtttgacctgcctcttttatttgatgcatgacttctcagagaa

SEQ ID NO: 54 - DNA, 362 bp, homo sapiens
ctcttcagttgcccactattattgcttatttttccttttcttgtctatcagtcaccttgaaactggtaatctgattcaagttaaacaatgttccttttgaatctagaaaacaagagaaatgcaaagtcattattccctcattctatgcttccatttactctaagaattcagaaacaaacatgtgggtaacttcctgttatcttaaaaaaagaatcatcccttcggtattcccttaactatctggaacttgtactgtcattttataatttaccatgtgacataattgtttgacctgcctcttttatttgatgcatgacttctcagagaacctgttatcaactcactgtgtaaaaccacgatga

SEQ ID NO: 55 - DNA, 236 bp, homo sapiens
tttttctcttcagttgcccgctattattgcttatttttccttttcttgtctatcagtcaccttgaaactggtaatctgattcaagttaaacaatgttccttttgaatctagaaaacaagagaaatgcaaagtcattattccctcattctatgcttccatttactctaagaattcagaaacaaacatgtgggtaacttcctgttatcttaaaaaaagaatcatcccttcggtcgacg

SEQ ID NO: 56 - DNA, 368 bp, homo sapiens
agaaacagtctttctgtgtatacctacggatgagggtattatttaaactgccaacaatatccaagacatggtcaataacctaattataaatactttagaaagagtgaccaggacatgtatagaaatgtctgcttacctgtagactttaaaaacaaacaaaaaaaacaaacaaaatttttggagcatttaatcattttttttctccttttatctcctttgtaatcttattgtctcctgagtaaatatacacataaatgtttggggattcattgctgctagattatatcaggtgtttacatagtgtctactatatgctgttgataagctttttcctaaaaatagttatcctcttttgtagtgtttttccc

SEQ ID NO: 57 - DNA, 153 bp, homo sapiens
tttttttttgtctatcagtcaccttgaaactggtaatctgattcaagttaaacaatgttccttttgaatctagaaaacaagagaaatgcaaagtcattattccctcattctatgcttccatttactctaagaattcagaaacaaacatgtggg

SEQ ID NO: 58 - DNA, 324 bp, homo sapiens
agaaaacagtctttctgygtatacctacggatsagggtattatttaaactgccamcaatatccaagvcatggtcaataacctaadcataamtactttagaaagagtgaccaggccatgtatagaaatgtctgcttactgtagactttaaaaacaaacaaaaaaacaaacaaatthttggagcatttaatcattthttttctccttttatctcctthtgtaatcttattgtctcctgagtaaatatacacataaatstttkgggattcattgctgbhagattatatcaggtgtttacatagtgtctactatatgctgttgataag

SEQ ID NO: 59 - DNA, 416 bp, homo sapiens
gtctatcagtcaccttgaaactggtaatctgattcaagttaaacaatgttccttttgaatctagaaaacaagagaaatgcaaagtcattattccctcattctatgcttccatttactctaagaattcagaaacaaacatgtgggtaacttcctgttatcttaaaaaaagaatcatcccttcggtattcccttaactatctggaacttgtactgtcattttataatttaccatgtgacataattgtttgacctgcctcttttatttgatgcatgacttctcagagaacctgttatcaactcactgtgtaaaaccacgatgaaatgaaggataactgatcacaaagaattatgtcttttgagatccaacaaatttacaaattataagagaaaaatgcaattttttaaaaaaggatatc

SEQ ID NO: 60 - DNA, 2489 bp, homo sapiens
ctccgccgcgggagggagctgcggctgtgccggccgagcgggggagggcgccgccactcagagccagggagggagccgctggagcggaagcccggaggccgcgctgcgccggggtgaggtggctttgaccccgggttgcccggccagcacgaccgaggaggtggctggacagctggaggatgaacggagaagccgactgccccacagacctggaaatggccgcccccaaaggccaagaccgttggtcccaggaagacatgctgactttgctggaatgcatgaagaacaaccttccatccaatgacagctccaagttcaaaaccaccgaatcacacatggactgggaaaaagtagcatttaaagacttttctggagacatgtgcaagctcaaatgggtggagatttctaatgaggtgaggaagttccgtacattgacagaattgatcctcgatgctcaggaacatgttaaaaatccttacaaaggcaaaaaactcaagaaacacccagacttcccaaagaagcccctgaccccttatttccgcttcttcatggagaagcgggccaagtatgcgaaactccaccctgagatgagcaacctggacctaaccaagattctgtccaagaaatacaaggagcttccggagaagaagaagatgaaatatattcaggacttccagagagagaaacaggagttcgagcgaaacctggcccgattcagggaggatcaccccgacctaatccagaatgccaagaaatcggacatcccagagaagcccaaaaccccccagcagctgtggtacacccacgagaagaaggtgtatctcaaagtgcggccagatgagatcatgagagactatatccagaagcacccagagctgaacatcagtgaggagggtatcaccaagtccaccctcaccaaggccgaacgccagctcaaggacaagtttgacgggcgacccaccaagccacctccgaacagctactcgctgtactgcgcagagctcatggccaacatgaaggacgtgcccagcacagagcgcatggtgctgtgcagccagcagtggaagctgctgtcccagaaggagaaggacgcctatcacaagaagtgtgatcagaaaaagaaagattacgaggtggagctgctccgtttcctcgagagcctgcctgaggaggagcagcagcgggtcttgggggaagagaagatgctgaacatcaacaagaagcaggccaccagccccgcctccaagaagccagcccaggaagggggcaagggcggctccgagaagcccaagcggcccgtgtcggccatgttcatcttctcggaggagaaacggcggcagctgcaggaggagcggcctgagctctccgagagcgagctgacccgcctgctggcccgaatgtggaacgacctgtctgagaagaagaaggccaagtacaaggcccgagaggcggcgctcaaggctcagtcggagaggaagcccggcggggagcgcgaggaacggggcaagctgcccgagtcccccaaaagagctgaggagatctggcaacagagcgttatcggcgactacctggcccgcttcaagaatgaccgggtgaaggccttgaaagccatggaaatgacctggaataacatggaaaagaaggagaaactgatgtggattaagaaggcagccgaagaccaaaagcgatatgagagagagctgagtgagatgcgggcacctccagctgctacaaattcttccaagaagatgaaattccagggagaacccaagaagcctcccatgaacggttaccagaagttctcccaggagctgctgtccaatggggagctgaaccacctgccgctgaaggagcgcatggtggagatcggcagtcgctggcagcgcatctcccagagccagaaggagcactacaaaaagctggccgaggagcagcaaaagcagtacaaggtgcacctggacctctgggttaagagcctgtctccccaggaccgtgcagcatataaagagtacatctccaataaacgtaagagcatgaccaagctgcgaggcccaaaccccaaatccagccggactactctgcagtccaagtcggagtccgaggaggatgatgaagaggatgaggatgacgaggacgaggatgaagaagaggaagatgatgagaatggggactcctctgaagatggcggcgactcctctgagtccagcagcgaggacgagagcgaggatggggatgagaatgaagaggatgacgaggacgaagacgacgacgaggatgacgatgaggatgaagataatgagtccgagggcagcagctccagctcctcctcctcaggggactcctcagactctgactccaactgaggctcagccccaccccagggcagccagggagagcccaggagctcccctccccaactgaccacctttgtttctcccccatgttctgtcccttgcccccctggcctcccccactttctttctttcttt

SEQ ID NO: 61 - protein, 727 aa, homo sapiens
MNGEADCPTDLEMAAPKGQDRWSQEDMLTLLECMKNNLPSNDSSKFKTTESHMDWEKVAFKDFSGDMCKLKWVEISNEVRKFRTLTELILDAQEHVKNPYKGKKLKKHPDFPKKPLTPYFRFFMEKRAKYAKLHPEMSNLDLTKILSKKYKELPEKKKMKYIQDFQREKQEFERNLARFREDHPDLIQNAKKSDIPEKPKTPQQLWYTHEKKVYLKVRPDEIMRDYIQKHPELNISEEGITKSTLTKAERQLKDKFDGRPTKPPPNSYSLYCAELMANMKDVPSTERMVLCSQQWKLLSQKEKDAYHKKCDQKKKDYEVELLRFLESLPEEEQQRVLGEEKMLNINKKQATSPASKKPAQEGGKGGSEKPKRPVSAMFIFSEEKRRQLQEERPELSESELTRLLARMWNDLSEKKKAKYKAREAALKAQSERKPGGEREERGKLPESPKRAEEIWQQSVIGDYLARFKNDRVKALKAMEMTWNNMEKKEKLMWIKKAAEDQKRYERELSEMRAPPAATNSSKKMKFQGEPKKPPMNGYQKFSQELLSNGELNHLPLKERMVEIGSRWQRISQSQKEHYKKLAEEQQKQYKVHLDLWVKSLSPQDRAAYKEYISNKRKSMTKLRGPNPKSSRTTLQSKSESEEDDEEDEDDEDEDEEEEDDENGDSSEDGGDSSESSSEDESEDGDENEEDDEDEDDDEDDDEDEDNESEGSSSSSSSSGDSSDSDSN

SEQ ID NO: 62 - DNA, 607 bp, homo sapiens
  misc_feature 602: n = A,T,C or G
ttttcagcatgagaatatgtgaatatgtttatttaggtttaacttacttcttactatatagatttggcttgttttttataataacaactgatatatgattcacaaaaaagcagagaagagtaagagaaagagagagaaatggagaaagagaagaaaaaagggataaagaatgaaagagagaaagagaataccattctctaaaggaagaggtgcagaaaattccattatcctttcttcttgatcatgccttgtatgattggcagccaaactagcccactgtgaaacccaacgtttgcttccagatgaagatgtgccttcctctgagtggtgaaatccagatgtagtcagtggttttctttcttccattactgctgcagcagaactgagagcccaatcttttattagatctttatgtttttcgttgataacaggcctattataatccgattgtcatctactccaaacacaacagctggtctgatgctttcagtagccggacctctgtagcttttgtgttcgaatggtggcgtctaagtgttcctcaagagttgcacgtttgctacagcgccgtgagccccagcgttctctgaatcacttgcgtncatca

SEQ ID NO: 63 - DNA, 402 bp, homo sapiens
  misc_feature 35: n = A,T,C or G
ggcagagcacagaccaagccaggagatggataaangttaaaaaatcaagcaacttctgctacttctgaaaaggataatgatgatgaccaaagtgacaagggtacttataccattgagttagagaatcccaacagtgaggaagtggaagcaagaaaaatgattcacaaggtaaataattgaaatttgagtgtgatcttagttgttgtgtggtgtatttgactggtggaaattattggagagtcagcatgagatgttgtcatgcagtcagtggtatgtgaattttagggttttattagggaactgcaagactaacagtaagaccaacatgctttgtgattttatttgctgatattctgaatttacctgagtttcatacataaagctctgtacatttaaaaggtt

SEQ ID NO: 64 - DNA, 607 bp, homo sapiens; identical to SEQ ID NO: 62
  misc_feature 602: n = A,T,C or G

SEQ ID NO: 65 - DNA, 317 bp, homo sapiens
  misc_feature 17, 25, 37, 41, 53, 68, 70, 144: n = A,T,C or G
tggggcgtgtgtggaanaacgttantgcccagcggantagnggccccggagcncgaccgcagcggcanancgacaacagcggcgacgacgacgacgacgaggtggggggaggacggcgtgcgagagactcacgggacgcgacgnccccgcctcccccgtccggtccctctctccacggtaaggggatgacgtagctttgccaaagacttagaagctaagcagaaaatgagcttaacatcctggtttttggtgagcagtggaggcactcgccacaggctgccacgagaaatgatttttgttggaaaaaatgactgtga

SEQ ID NO: 66 - DNA, 420 bp, homo sapiens
gtccctgaagaagctcttaaggtaacagtttttacttaacttcttttgcaaatctactcttcactatggttgattttacttcttgatgtttcacttccatttttaaatgttttatagcatgagaagtttaccattcagcttcagttgtcccaaaaatcttcagaatcagaattatccaaatctgcaagtgccaaaagcatagattcaaaggtagcagacgctgctactgaagtgcagcacaaaactactgaagcactgaaatccgaggaaaaagccatgggtaagctggctctctcgaaagacatctttatactgatcttcgaagacactgcatgcttgtctcagaaagtgctatgtccattaaaatattatatagtgatatcagagtgtgtttatgctaccagtgcttcatagacatat

SEQ ID NO: 67 - DNA, 7497 bp, homo sapiens
gcgcaagaggatcagggatagcctctgagctcgggttcccagggttcgtagcttccaacggctgcgcgcgcacttcggtcgcgggcggtgaggtgctgttgctgaaacgctgccgctgagggtggactcgatttcccagggtcccgccgcgggagtctccggcgggcgggcgcgcgcgagccaccgagcgaggtgatagaggcggcggcccaggcgtctgggtcctgctggtcttcgcctttcttctccgcttctaccccgtcggccgctgccactggggtccctggccccaccgacatggcggcggtgttgcagcaagtcctggagcgcacggagctgaacaagctgcccaagtctgtccagaacaaacttgaaaagttccttgctgatcagcaatccgagatcgatggcctgaaggggcggcatgagaaatttaaggtggagagcgaacaacagtattttgaaatagaaaagaggttgtcccacagtcaggagagacttgtgaatgaaacccgagagtgtcaaagcttgcggcttgagctagagaaactcaacaatcaactgaaggcactaactgagaaaaacaaagaacttgaaattgctcaggatcgcaatattgccattcagagccaatttacaagaacaaaggaagaattagaagctgagaaaagagacttaattagaaccaatgagagactatctcaagaacttgaatacttaacagaggatgttaaacgtctgaatgaaaaacttaaagaaagcaatacaacaaagggtgaacttcagttaaaattggatgaacttcaagcttctgatgtttctgttaagtatcgagaaaaacgcttggagcaagaaaaggaattgctacatagtcagaatacatggctgaatacagagttgaaaaccaaaactgatgaacttctggctcttggaagagaaaaagggaatgagattctagagcttaaatgtaatcttgaaaataaaaaagaagaggtttctagactggaagaacaaatgaatggcttaaaaacatcaaatgaacatcttcaaaagcatgtggaggatctgttgaccaaattaaaagaggccaaggaacaacaggccagtatggaagagaaattccacaatgaattaaatgcccacataaaactttctaatttgtacaagagtgccgctgatgactcagaagcaaagagcaatgaactaacccgggcagtagaggaactacacaaacttttgaaagaagctggtgaagccaacaaagcaatacaagatcatcttctagaggtggagcaatccaaagatcaaatggaaaaagaaatgcttgagaaaatagggagattggagaaggaattagagaatgcaaatgaccttctttctgccacaaaacgtaaaggagccatattgtctgaagaagagcttgccgccatgtctcctactgcagcagctgtagctaagatagtgaaacctgggatgaaactaactgagctctataatgcttatgtggaaactcaggatcagttgcttttggagaaactagagaacaaaagaattaataagtacctagatgaaatagtgaaagaagtggaagccaaagcaccaattttgaaacgccagcgtgaggaatatgaacgtgcacagaaagctgtagcaagtttatctgttaagcttgaacaagctatgaaggagattcagcgattgcaggaggacactgataaagccaacaagcaatcatctgtacttgagagagataatcgaagaatggaaatacaagtaaaagatctttcacaacagattagagtgcttttgatggaacttgaagaagcaaggggtaaccacgtaattcgtgatgaggaagtaagctctgctgatataagtagttcatctgaggtaatatcacagcatctagtatcttacagaaatattgaagagcttcaacaacaaaatcaacgtctcttagtggcccttagagagcttggggaaaccagagaaagagaagaacaagaaacaacttcatccaaaatcactgagcttcagctcaaacttgagagtgcccttactgaactagaacaactccgcaaatcacgacagcatcaaatgcagcttgttgattccatagttcgtcagcgtgatatgtaccgtattttattgtcacaaacaacaggagttgccattccattacatgcttcaagcttagatgatgtttctcttgcatcaactccaaaacgtccaagtacatcacagactgtttccactcctgctccagtacctgttattgaatcaacagaggctatagaggctaaggctgcccttaaacagttgcaggaaatttttgagaactacaaaaaagaaaaagcagaaaatgaaaaaatacaaaatgagcagcttgagaaacttcaagaacaagttacagatttgcgatcacaaaataccaaaatttctacccagctagattttgcttctaaacgttatgaaatgctgcaagataatgttgaaggatatcgtcgagaaataacatcacttcatgagagaaatcagaaactcactgccacaactcaaaagcaagaacagattatcaatacgatgactcaagatttgagaggagcaaatgagaagctagctgtcgcagaagtaagagcagaaaatttgaagaaggaaaaggaaatgcttaaattgtctgaagttcgtctttctcagcaaagagagtctttgttagctgaacaaagggggcaaaacttactgctaactaatctgcaaacaattcagggaatactggagcgatctgaaacagaaaccaaacaaaggcttagtagccagatagaaaaactggaacatgagatctctcatctaaagaagaagttggaaaatgaggtggaacaaaggcatacacttactagaaatctagatgttcaacttttagatacaaagagacaactggatacagagacaaatcttcatcttaacacaaaagaactattaaaaaatgctcaaaaagaaattgccacattgaaacagcacctcagtaatatggaagtccaagttgcttctcagtcttcacagagaactggtaaaggtcagcctagcaacaaagaagatgtggatgatcttgtgagtcagctaagacagacagaagagcaggtgaatgacttaaaggagagactcaaaacaagtacgagcaatgtggaacaatatcaagcaatggttactagtttagaagaatccctgaacaaggaaaaacaggtgacagaagaagtgcgtaagaatattgaagttcgtttaaaagagtcagctgaatttcagacacagttggaaaagaagttgatggaagtagagaaggaaaaacaagaacttcaggatgataaaagaagagccatagagagcatggaacaacagttatctgaattgaagaaaacactttctagtgttcagaatgaagtacaagaagctcttcagagagcaagcacagctttaagtaatgagcagcaagccagacgtgactgtcaggaacaagctaaaatagctgtggaagctcagaataagtatgagagagaattgatgctgcatgctgctgatgttgaagctctacaagctgcgaaggagcaggtttcaaaaatggcatcagtccgtcagcatttggaagaaacaacacagaaagcagaatcacagttgttggagtgtaaagcatcttgggaggaaagagagagaatgttaaaggatgaagtttccaaatgtgtatgtcgctgtgaagatctggagaaacaaaacagattacttcatgatcagatcgaaaaattaagtgacaaggtcgttgcctctgtgaaggaaggtgtacaaggtccactgaatgtatctctcagtgaagaaggaaaatctcaagaacaaattttggaaattctcagatttatacgacgagaaaaagaaattgctgaaactaggtttgaggtggctcaggttgagagtctgcgttatcgacaaagggttgaacttttagaaagagagctgcaggaactcgaagatagtctaaatgctgaaagggagaaagtccaggtaactgcaaaaacaatggctcagcatgaagaactgatgaagaaaactgaaacaatgaatgtagttatggagaccaataaaatgctaagagaagagaaggagagactagaacaggatctacagcaaatgcaagcaaaggtgaggaaactggagttagatattttacccttacaagaagcaaatgctgagctgagtgagaaaagcggtatgttgcaggcagagaagaagctcttagaagaggatgtcaaacgttggaaagcacgtaaccagcatctagtaagtcaacagaaagatccagatacagaagaatatcggaagctcctttctgaaaaggaagttcatactaagcgtattcaacaattgacagaagaaattggtagacttaaagctgaaattgcaagatcaaatgcatctttgactaacaaccagaacttaattcagagtctgaaggaagatctaaataaagtaagaactgaaaaggaaaccatccagaaggacttagatgccaaaataattgatatccaagaaaaagtcaaaactattactcaagttaagaaaattggacgtaggtacaagactcaatatgaagaacttaaagcacaacaggataaggttatggagacatcggctcagtcctctggagaccatcaggagcagcatgtttcagtccaggaaatgcaggaactcaaagaaacgctcaaccaagctgaaacaaaatcaaaatcacttgaaagtcaagtagagaatctgcagaagacattatctgaaaaagagacagaagcaagaaatctccaggaacagactgtgcaacttcagtctgaactttcacgacttcgtcaggatcttcaagatagaaccacacaggaggagcagctccgacaacagataactgaaaaggaagaaaaaaccagaaaggctattgtagcagcaaagtcaaaaattgcacacttagctggtgtaaaagatcagctaactaaagaaaatgaggagcttaaacaaaggaatggagccttagatcagcagaaagatgaattggatgttcgcattactgcgctaaagtcccaatatgaaggtcgaattagtcgcttggaaagagaactcagggagcatcaagagagacaccttgagcagagagatgagcctcaagaaccttctaataaggtccctgaacagcagagacagatcacattgaaaacaactccagcttctggtgaaagaggaattgccagcacatcagacccaccaacagccaatatcaagccaactcctgttgtgtctactccaagtaaagtgacagctgcagctatggctggaaataagtcaacacccagggctagtatccgcccaatggttacacctgcaactgttacaaatcccactactaccccaacagctacagtgatgcccactacacaagtggaatcacaggaagctatgcagtcagaagggcctgtggaacatgttccagtttttggaagcacaagtggatccgttcgttctactagtcctaatgtccagccttctatctctcaacctattttaactgttcagcaacaaacacaggctacagcttttgtgcaacccactcaacagagtcatcctcagattgagcctgccaatcaagagttatcttcaaacatagtagaggttgttcagagttcaccagttgagcggccttctacttccacagcagtatttggcacagtttcggctacccccagttcttctttgccaaagcgtacacgtgaagaggaagaggatagcaccatagaagcatcagaccaagtctctgatgatacagtggaaatgcctcttccaaagaagttgaaaagtgtcacacctgtaggaactgaggaagaagttatggcagaagaaagtactgatggagaggtagagactcaggtatacaaccaggattctcaagattccattggagaaggagttacccagggagattatacacctatggaagacagtgaagaaacctctcagtctctacaaatagatcttgggccacttcaatcagatcagcagacgacaacttcatcccaggatggtcaaggcaaaggagatgatgtcattgtaattgacagtgatgatgaagaagaggatgaggaagatgatgatgatgatgaagatgacacagggatgggagatgagggtgaagatagtaatgaaggaactggtagtgccgatggcaatgatggttatgaagctgatgatgctgagggtggtgatgggactgatccaggtacagaaacagaagaaagtatgggtggaggtgaaggtaatcacagagctgctgattctcaaaacagtggtgaaggaaatacaggtgctgcagaatcttctttttctcaggaggtttctagagaacaacagccatcatcagcatctgaaagacaggcccctcgagcacctcagtcaccgagacgcccaccacatccacttcccccaagactgaccattcatgccccacctcaggagttgggaccaccagttcagagaattcagatgacccgaaggcagtctgtaggacgtggccttcagttgactccaggaataggtggcatgcaacagcatttttttgatgatgaagacagaacagttccaagtactccaactcttgtggtgccacatcgtactgatggatttgctgaagcaattcattcgccgcaggttgctggtgtccctagattccggtttgggccacctgaagatatgccacaaacaagttctagtcactctgatcttggccagcttgcttctcaaggaggtttaggaatgtatgaaacacccctgttcctagctcatgaagaagagtcaggtggccgaagtgttcccactactccactacaagtagcagccccagtgactgtatttactgagagcaccacctctgatgcttcggaacatgcctctcaatctgttccaatggtgactacatccactggcactttatctacaacaaatgaaacagcaacaggtgatgatggagatgaagtatttgtggaggcagaatctgaaggtattagttcagaagcaggcctagaaattgatagccagcaggaagaagagccggttcaagcatctgatgagtcagatctcccctccaccagccaggatcctccttctagctcatctgtagatactagtagtagtcaaccaaagcctttcagacgagtaagacttcagacaacattgagacaaggtgtccgtggtcgtcagtttaacagacagagaggtgtgagccatgcaatgggagggagaggaggaataaacagaggaaatattaattaaatggtctgtaaacaataacaactgtgaataagattatcaaatctgttttagtgtaatgattgtcaagtttaaaaacatttttatatataaactggtatactcatgtcaatattctttattaataaaatgtttttcagtgtcaaaaaaaaa

SEQ ID NO: 68 - protein, 2349 aa, homo sapiens
MAAVLQQVLERTELNKLPKSVQNKLEKFLADQQSEIDGLKGRHEKFKVESEQQYFEIEKRLSHSQERLVNETRECQSLRLELEKLNNQLKALTEKNKELEIAQDRNIAIQSQFTRTKEELEAEKRDLIRTNERLSQELEYLTEDVKRLNEKLKESNTTKGELQLKLDELQASDVSVKYREKRLEQEKELLHSQNTWLNTELKTKTDELLALGREKGNEILELKCNLENKKEEVSRLEEQMNGLKTSNEHLQKHVEDLLTKLKEAKEQQASMEEKFHNELNAHIKLSNLYKSAADDSEAKSNELTRAVEELHKLLKEAGEANKAIQDHLLEVEQSKDQMEKEMLEKIGRLEKELENANDLLSATKRKGAILSEEELAAMSPTAAAVAKIVKPGMKLTELYNAYVETQDQLLLEKLENKRINKYLDEIVKEVEAKAPILKRQREEYERAQKAVASLSVKLEQAMKEIQRLQEDTDKANKQSSVLERDNRRMEIQVKDLSQQIRVLLMELEEARGNHVIRDEEVSSADISSSSEVISQHLVSYRNIEELQQQNQRLLVALRELGETREREEQETTSSKITELQLKLESALTELEQLRKSRQHQMQLVDSIVRQRDMYRILLSQTTGVAIPLHASSLDDVSLASTPKRPSTSQTVSTPAPVPVIESTEAIEAKAALKQLQEIFENYKKEKAENEKIQNEQLEKLQEQVTDLRSQNTKISTQLDFASKRYEMLQDNVEGYRREITSLHERNQKLTATTQKQEQIINTMTQDLRGANEKLAVAEVRAENLKKEKEMLKLSEVRLSQQRESLLAEQRGQNLLLTNLQTIQGILERSETETKQRLSSQIEKLEHEISHLKKKLENEVEQRHTLTRNLDVQLLDTKRQLDTETNLHLNTKELLKNAQKEIATLKQHLSNMEVQVASQSSQRTGKGQPSNKEDVDDLVSQLRQTEEQVNDLKERLKTSTSNVEQYQAMVTSLEESLNKEKQVTEEVRKNIEVRLKESAEFQTQLEKKLMEVEKEKQELQDDKRRAIESMEQQLSELKKTLSSVQNEVQEALQRASTALSNEQQARRDCQEQAKIAVEAQNKYERELMLHAADVEALQAAKEQVSKMASVRQHLEETTQKAESQLLECKASWEERERMLKDEVSKCVCRCEDLEKQNRLLHDQIEKLSDKVVASVKEGVQGPLNVSLSEEGKSQEQILEILRFIRREKEIAETRFEVAQVESLRYRQRVELLERELQELEDSLNAEREKVQVTAKTMAQHEELMKKTETMNVVMETNKMLREEKERLEQDLQQMQAKVRKLELDILPLQEANAELSEKSGMLQAEKKLLEEDVKRWKARNQHLVSQQKDPDTEEYRKLLSEKEVHTKRIQQLTEEIGRLKAEIARSNASLTNNQNLIQSLKEDLNKVRTEKETIQKDLDAKIIDIQEKVKTITQVKKIGRRYKTQYEELKAQQDKVMETSAQSSGDHQEQHVSVQEMQELKETLNQAETKSKSLESQVENLQKTLSEKETEARNLQEQTVQLQSELSRLRQDLQDRTTQEEQLRQQITEKEEKTRKAIVAAKSKIAHLAGVKDQLTKENEELKQRNGALDQQKDELDVRITALKSQYEGRISRLERELREHQERHLEQRDEPQEPSNKVPEQQRQITLKTTPASGERGIASTSDPPTANIKPTPVVSTPSKVTAAAMAGNKSTPRASIRPMVTPATVTNPTTTPTATVMPTTQVESQEAMQSEGPVEHVPVFGSTSGSVRSTSPNVQPSISQPILTVQQQTQATAFVQPTQQSHPQIEPANQELSSNIVEVVQSSPVERPSTSTAVFGTVSATPSSSLPKRTREEEEDSTIEASDQVSDDTVEMPLPKKLKSVTPVGTEEEVMAEESTDGEVETQVYNQDSQDSIGEGVTQGDYTPMEDSEETSQSLQIDLGPLQSDQQTTTSSQDGQGKGDDVIVIDSDDEEEDEEDDDDDEDDTGMGDEGEDSNEGTGSADGNDGYEADDAEGGDGTDPGTETEESMGGGEGNHRAADSQNSGEGNTGAAESSFSQEVSREQQPSSASERQAPRAPQSPRRPPHPLPPRLTIHAPPQELGPPVQRIQMTRRQSVGRGLQLTPGIGGMQQHFFDDEDRTVPSTPTLVVPHRTDGFAEAIHSPQVAGVPRFRFGPPEDMPQTSSSHSDLGQLASQGGLGMYETPLFLAHEEESGGRSVPTTPLQVAAPVTVFTESTTSDASEHASQSVPMVTTSTGTLSTTNETATGDDGDEVFVEAESEGISSEAGLEIDSQQEEEPVQASDESDLPSTSQDPPSSSSVDTSSSQPKPFRRVRLQTTLRQGVRGRQFNRQRGVSHAMGGRGGINRGNIN

(19) United States
(12) Patent Application Publication
(10) Pub. No.: US 2008/0044418 A1
(43) Pub. Date: Feb. 21, 2008

(54) Tumor Antigens and Uses Thereof

(76) Inventors: Glenn Dranoff, Lexington, MA (US); Jan Schmollinger, Berlin (DE); F. Stephen Hodi, Framingham, MA (US); Joseph Mollick, Palo Alto, CA (US)

Correspondence Address: BROMBERG & SUNSTEIN LLP, 125 SUMMER STREET, BOSTON, MA, 02110-1618, US

(21) Appl. No.: 11/782,310

(22) Filed: Jul. 24, 2007

Related U.S. Application Data
(62) Division of application No. 09/762,577, filed on Aug. 29, 2002, now Pat. No. 7,250,291.
(60) Provisional application No. 60/095,766, filed on Aug. 7, 1998.

(51) Int. Cl. A61K 39/395; A61K 31/70; A61P 43/00; C07K 16/18; C12N 15/11; C12Q 1/68; G01N 33/53; G01N 33/48; C12N 5/06; C12N 15/00; C07K 14/00; A61K 39/00

(52) U.S. Cl. 424/138.1; 424/178.1; 424/184.1; 424/185.1; 435/320.1; 435/325; 435/455; 435/6; 435/7.23; 435/7.24; 436/86; 514/44; 530/350; 530/387.9; 536/23.5

(57) ABSTRACT

The invention features tumor antigens; tumor antigen-encoding nucleic acids; antibodies specific for tumor antigens and methods of using the antibodies; methods of identifying tumor antigens and the nucleic acids that encode them; methods of monitoring or diagnosing tumors in patients; methods of testing patients for the increased likelihood of developing a tumor; and methods and compositions for treatment of a tumor or prophylaxis against developing a tumor.

### CROSS REFERENCE TO RELATED APPLICATIONS

This application is a Divisional Application of U.S. National Stage application Ser. No. 09/762,577, filed under 35. U.S.C. § 371 (f), of International Application No. PCT/US99/17738 filed Aug. 8, 1999, which claims priority from U.S. Provisional Application Ser. No. 60/095,766 filed Aug. 7, 1998, the contents of which are both hereby incorporated by reference herein.

### REPLACEMENT SEQUENCE LISTING APPENDIX

The sequences described in the specification, namely SEQ ID NO:1 through SEQ ID NO:68, and disclosed in the “Replacement Sequence Listing” are being submitted with this application via the USPTO electronic filing system (EFS) in a text file named “2486-113ReplacementSeqListCopyl.txt” (prepared Jul. 23, 2007-156 kb total) in compliance with 37 CFR. §1.52(e) and §1.821(c) and are hereby incorporated by reference herein in their entirety.

### BACKGROUND OF THE INVENTION

There is compelling evidence that malignant melanoma cells evoke specific humoral and cellular anti-tumor immune responses in some patients. For example, the radial growth phase of primary melanoma is regularly associated with a significant dermal lymphocytic reaction that often results in partial tumor destruction. Moreover, clonal expansion of T cells occurs in primary regressing melanoma; lymphocytes explanted from such lesions demonstrate cytotoxicity towards autologous melanoma cells in vitro.

Although a brisk lymphocytic infiltrate in the vertical growth phase of primary melanoma occurs infrequently, this response is tightly correlated with prolonged survival and a reduced incidence of metastatic disease. Melanoma that has spread to regional lymph nodes may occasionally elicit a striking lymphocytic reaction which is also highly associated with improved survival. In rare cases, widely disseminated melanoma may undergo spontaneous regression accompanied by a diffuse infiltrate of lymphocytes, plasma cells, and macrophages. Notwithstanding these provocative findings, however, it is clear that most patients fail to develop anti-melanoma immune responses that are sufficiently potent to prevent lethal tumor progression.

The application of gene transfer technologies to investigative efforts in tumor immunology has led to the development of several novel strategies to enhance the frequency and intensity of anti-tumor immune responses. A large number of pre-clinical studies have convincingly demonstrated that engineering murine tumor cells to express a variety of immunostimulatory molecules can lead to enhanced tumor immunogenicity. Among the approaches utilizing ex vivo modification of tumor cells, we have shown that vaccination with irradiated tumor cells engineered to secrete granulocyte-macrophage colony stimulating factor (GM-CSF) stimulates potent, specific, and long-lasting anti-tumor immunity in multiple murine tumor model systems, including malignant melanoma (Dranoff et al., Proc. Natl. Acad. Sci., U.S.A., 90:3539-3543, 1993). Immunization requires the participation of both CD4- and CD8-positive T lymphocytes and likely involves improved tumor antigen presentation by dendritic cells and macrophages recruited to the vaccination site. Identification of individual tumor antigens is likely to contribute to improved cancer diagnosis and treatment.

### SUMMARY OF THE INVENTION

Vaccination with irradiated, autologous melanoma cells engineered to secrete GM-CSF stimulates potent anti-tumor immunity in humans with metastatic melanoma. We established a melanoma cell line from a metastatic tumor removed from a vaccinated patient, and constructed a cDNA expression library from mRNA isolated from the melanoma cells. The library was screened with serum from vaccinated patients, and several tumor antigen clones were isolated.

In a first aspect, the invention features a method of identifying a nucleic acid encoding a tumor antigen or a fragment thereof, comprising: a) identifying a patient with a tumor;

b) obtaining tumor cells from the patient; c) vaccinating the patient with a vaccine preparation comprising the tumor cells together with a GM-CSF sustained delivery system to generate an immune response in the patient; and d) isolating, either from an autologous post-vaccination tumor sample obtained from the patient, or from allogeneic tumor cells, nucleic acid that encodes a tumor antigen or a fragment thereof, wherein the nucleic acid encoding the tumor antigen or fragment is detected by an antibody in serum obtained from the patient, wherein the antibody specifically binds the tumor antigen.

In a second aspect, the invention features a method of identifying a nucleic acid encoding a tumor antigen or a fragment thereof, comprising: a) identifying a patient with a tumor; b) obtaining tumor cells from the patient; c) vaccinating the patient with a vaccine preparation comprising the tumor cells together with a GM-CSF sustained delivery system to generate an immune response in the patient; and d) isolating, either from an autologous post-vaccination tumor sample obtained from the patent, or from allogeneic tumor cells, nucleic acid that encodes the tumor antigen or the fragment thereof, wherein the nucleic acid encoding the tumor antigen or fragment is detected by a cytotoxic T lymphocyte obtained from the patent, wherein the cytotoxic T lymphocyte specifically binds the tumor antigen.

In a third aspect, the invention features a method of identifying a tumor antigen or a fragment thereof, comprising: a) identifying a patient with a tumor; b) obtaining tumor cells from the patient; c) vaccinating the patient with a vaccine preparation comprising the tumor cells together with a GM-CSF sustained delivery system to generate an immune response in the patient; and d) isolating the tumor antigen or the fragment thereof either from an autologous post-vaccination tumor sample obtained from the patient or from allogeneic tumor cells, wherein the tumor antigen or fragment is detected by an antibody in serum obtained from the patient, wherein the antibody specifically binds the tumor antigen.

In a fourth aspect, the invention features a method of identifying a tumor antigen or a fragment thereof, comprising: a) identifying a patient with a tumor; b) obtaining tumor cells from the patient; c) vaccinating the patient with a vaccine preparation comprising the tumor cells together with a GM-CSF sustained delivery system to generate an immune response in the patient; and d) isolating the tumor antigen or the fragment thereof from either an autologous post-vaccination tumor sample obtained from the patient or from allogeneic tumor cells, wherein the tumor antigen or the fragment is detected by a cytotoxic T lymphocyte obtained from the patient, wherein the cytotoxic T lymphocyte specifically binds the tumor antigen.

In preferred embodiments of the first, second, third, and fourth aspects of the invention, the GM-CSF sustained delivery system comprises a plasmid or viral expression vector encoding GM-CSF that is transfected or transduced into the tumor cells prior to vaccination, or the GM-CSF sustained delivery system comprises cells expressing GM-CSF that are mixed with the tumor cells prior to vaccination (for example, allogeneic cells or other cultured cells) or the GM-CSF sustained delivery system comprises microspheres releasing GM-CSF that are mixed with the tumor cells prior to vaccination.

In other embodiments of the first four aspects of the invention, the serum may be pre-vaccination serum, or post-vaccination serum, and detection by post-vaccination serum may be more sensitive than detection by pre-vaccination serum.

In still other embodiments of the first four aspects of the invention, the cells are irradiated prior to vaccination, the tumor may be a leukemia, a lymphoma, a brain tumor (e.g., a glioblastoma or a neuroblastoma), a melanoma, a sarcoma, or a carcinoma such as a uterine, cervical, testicular, liver, ovarian, lung (e.g., non-small cell lung), renal cell, colon, breast, prostate, or bladder carcinoma, and vaccination increases the number of T lymphocytes and/or plasma cells in the patient's tumor, relative to the number of T lymphocytes and/or plasma cells in the patient's tumor prior to the vaccination.

In yet other embodiments of the first four aspects of the invention the tumor antigen fragment contains at least 10 amino acids, preferably at least 15, 20, 25, or 30 amino acids, more preferably at least 50 amino acids. Most preferably, the entire tumor antigen is identified.

In a fifth aspect, the invention features a method of monitoring or diagnosing a tumor in a patient, comprising detecting or measuring, in a sample from the patient, a tumor antigen, a nucleic acid encoding a tumor antigen, an antibody that specifically binds a tumor antigen, or a cytotoxic T lymphocyte that specifically binds a tumor antigen. The tumor antigen is identified by the method of the first four aspects of the invention.

In preferred embodiments of the fifth aspect of the invention, the sample is selected from: a tumor or tissue biopsy, a lymph node, bone marrow, cells, blood, urine, stool, sputum, saliva, cerebrospinal fluid, or uterine tissue.

In a sixth aspect, the invention features a substantially pure polypeptide or fragment thereof, wherein the fragment is at least ten amino acids long, and wherein the polypeptide comprises a polypeptide substantially identical to a polypeptide encoded by a nucleic acid sequence selected from TRAAM (SEQ ID NOs: 1, 3, and 17); TPR/UBP3 (SEQ ID NO: 7); UBP3 (SEQ ID NO: 7); BRAP-2/H⁺-ATPase (SEQ ID NO: 8); KOO8-1 (SEQ ID NO: 9); MAIAP (SEQ ID NO: 11); Gene AS (SEQ ID NO: 16); BR-1 (SEQ ID NO: 14); and BR-2 (SEQ ID NO: 15). In a preferred embodiment, the polypeptide is a human polypeptide.

In a seventh aspect, the invention features a purified nucleic acid comprising a sequence encoding a polypeptide or a fragment thereof, wherein the fragment is at least ten amino acids long, and wherein the polypeptide comprises a polypeptide substantially identical to a polypeptide encoded by a nucleic acid sequence selected from TRAAM (SEQ ID NOs: 1, 3, and 17); TPR/UBP3 (SEQ ID NO: 7); UBP3 (SEQ ID NO: 7); BRAP-2/H⁺-ATPase (SEQ ID NO: 8); KOO8-1 (SEQ ID NO: 9); MAIAP (SEQ ID NO: 1); Gene AS (SEQ ID NO: 16); BR-1 (SEQ ID NO: 14); and BR-2 (SEQ ID NO: 15). In a preferred embodiment, the nucleic acid comprises a nucleotide sequence set forth in the seventh aspect of the invention.

In an eighth aspect, the invention features a purified nucleic acid having a nucleotide sequence that hybridizes under high stringency conditions to a probe comprising at least fourteen consecutive nucleotides that are complementary to: TRAAM; TPR/UBP3; UBP3; BRAP-2/H⁺-ATPase; KOO8-1; MAIAP; Gene AS; BR-1; and BR-2. In a ninth aspect, the invention features a purified nucleic acid comprising a probe, wherein the probe hybridizes under high stringency conditions to TRAAM; TPR/UBP3; UBP3; BRAP-2/H⁺-ATPase; KOO8-1; MAIAP; Gene AS; BR-1; or BR-2, wherein the probe has a nucleotide sequence complementary to at least 14 consecutive nucleotides of TRAAM; TPR/UBP3; UBP3; BRAP-2/H⁺-ATPase; KOO8-1; MAIAP; Gene AS; BR-1; or BR-2.

In preferred embodiments of the eighth and ninth aspects of the invention, the nucleic acid is DNA or RNA.

In a tenth aspect, the invention features a vector comprising a tumor antigen nucleic acid according to the ninth aspect of the invention.

In an eleventh aspect, the invention features a cell containing nucleic acid according to the seventh, eighth, ninth, and tenth aspects of the invention.

In a twelfth aspect, the invention features a substantially pure antibody that specifically binds a polypeptide or a fragment thereof, wherein the polypeptide comprises a polypeptide encoded by a nucleic acid sequence chosen from: TRAAM; TPR/UBP3; UBP3; BRAP-2/H⁺-ATPase; H⁺-ATPase; KOO8-1; MAIAP; Gene AS; BR-1; and BR-2.

In a thirteenth aspect, the invention features a method of generating an antibody that specifically binds a polypeptide or a fragment thereof, wherein the polypeptide comprises a polypeptide substantially identical to a polypeptide encoded by a nucleic acid sequence selected from the group consisting of TRAAM; TPR/UBP3; UBP3; BRAP-2/H⁺-ATPase; H⁺-ATPase; KOO8-1; MAIAP; Gene AS; BR-1; and BR-2, the method comprising administering the polypeptide, or fragment thereof, to an animal capable of generating an immune response, and isolating the antibody from the animal.

In a fourteenth aspect, the invention features a method of detecting the presence of a polypeptide or a fragment thereof in a biological sample, wherein the polypeptide comprises a polypeptide substantially identical to a polypeptide encoded by a nucleic acid selected from the group consisting of TRAAM; TPR/UBP3; UBP3; BRAP-2/H-ATPase; H⁺-ATPase; KOO8-1; MAIAP; Gene AS; BR-1; and BR-2, the method comprising contacting the sample with an antibody that specifically binds the polypeptide or a fragment thereof, and assaying for binding of the antibody to the polypeptide.

In a fifteenth aspect, the invention features a method of testing a patient for the presence of a tumor or an increased likelihood of developing a tumor, comprising: a) obtaining a sample from the patient, b) measuring the level of an antibody in the sample, wherein the antibody specifically binds a tumor antigen, wherein the tumor antigen comprises a polypeptide encoded by a nucleic acid selected from the group consisting of: TRAAM; TPR/UBP3; UBP3; BRAP-2/H⁺-ATPase; KOO8-1; MAIAP; Gene AS; BR-1; BR-2; KIAA0603; TPR; NOR-90; and BRAP-2, c) comparing the antibody level in the patient sample to the antibody level in a reference sample, wherein an increase in the antibody level in the patient sample, relative to the antibody level in the reference sample, indicates that the patient has a tumor or the increased likelihood of developing a tumor.

In a sixteenth aspect, the invention features a method of testing a patient for the presence of a tumor or an increased likelihood of developing a tumor, comprising: a) obtaining a sample from the patient, b) measuring the level of cytotoxic T lymphocytes in the sample, wherein the cytotoxic T lymphocytes specifically bind a tumor antigen, wherein the tumor antigen comprises a polypeptide encoded by a nucleic acid selected from the group consisting of: TRAAM; TPR/UBP3; UBP3; BRAP-2/H⁺-ATPase; KOO8-1; MAIAP; Gene AS; BR-1; BR-2; KIAA0603; TPR; NOR-90; and BRAP-2, c) comparing the cytotoxic T lymphocyte level in the patient sample to the cytotoxic T lymphocyte level in a reference sample, wherein an increase in the cytotoxic T lymphocyte level in the patient sample, relative to the cytotoxic T lymphocyte level in the reference sample, indicates the patient has a tumor or the increased likelihood of developing a tumor.

In preferred embodiments of the fifteenth and sixteenth aspects of the invention, the tumor may be a leukemia, a lymphoma, a brain tumor (e.g., a glioblastoma or a neuroblastoma), a melanoma, a sarcoma, or a carcinoma such as a uterine, cervical, testicular, liver, ovarian, lung (e.g., non-small cell lung), renal cell, colon, breast, prostate, or bladder carcinoma.

In a seventeenth aspect, the invention features a method of testing a patient for the presence of a tumor or the increased likelihood of developing a tumor, comprising: a) obtaining a sample from the patient, b) measuring the level of a tumor antigen in the sample, wherein the tumor antigen comprises a polypeptide substantially identical to a polypeptide encoded by a nucleic acid selected from the group consisting of: TRAAM; TPR/UBP3; UBP3; BRAP-2/H⁺-ATPase; H⁺-ATPase; KOO8-1; MAIAP; Gene AS; BR-1; BR-2; KIAA0603; TPR; NOR-90; and BRAP-2, c) comparing the tumor antigen level in the patient sample to the tumor antigen level in a reference sample, wherein an increase in the tumor antigen level in the patient sample, relative to the tumor antigen level in the reference sample, indicates that the patient has a tumor or the increased likelihood of developing a tumor.

In preferred embodiments of the seventeenth aspect of the invention, the tumor antigen level may be measured by measuring the level of tumor antigen polypeptide, or by measuring the level of nucleic acid encoding the tumor antigen. The nucleic acid may be genomic DNA, mRNA, or cDNA.

In other embodiments of the seventeenth aspect, the sample may be selected from: a tumor or tissue biopsy, a lymph node, bone marrow, cells, blood, urine, stool, sputum, saliva, cerebrospinal fluid, or uterine tissue.

In still other embodiments of the seventeenth aspect, the tumor may be a leukemia, a lymphoma, a brain tumor (e.g., a glioblastoma or a neuroblastoma), a melanoma, a sarcoma, or a carcinoma such as a uterine, cervical, testicular, liver, ovarian, lung (e.g., non-small cell lung), renal cell, colon, breast, prostate, or bladder carcinoma.

In an eighteenth aspect, the invention features a method of determining the level of an antibody in a patient, wherein the antibody specifically binds a tumor antigen polypeptide comprising a polypeptide encoded by a nucleic acid selected from the group consisting of: TRAAM; TPR/UBP3; UBP3; BRAP-2/H⁺-ATPase; H⁺-ATPase; KOO8-1; MAIAP; Gene AS; BR-1; BR-2; KIAA0603; TPR; NOR-90; and BRAP-2, comprising: a) obtaining a sample containing the antibody from the patient, and b) measuring the level of the antibody in the patient sample, compared to a reference sample.

In a nineteenth aspect, the invention features a method of determining the level of cytotoxic T lymphocytes in a patient, wherein the cytotoxic T lymphocytes specifically bind a tumor antigen polypeptide comprising a polypeptide encoded by a nucleic acid selected from the group consisting of: TRAAM; TPR/UBP3; UBP3; BRAP-2/H⁺-ATPase; H⁺-ATPase; KOO8-1; MAIAP; Gene AS; BR-1; BR-2; KIAA0603; TPR; NOR-90; and BRAP-2, comprising: a) obtaining a sample containing the cytotoxic T lymphocytes from the patient, and b) measuring the level of cytotoxic T lymphocytes in the patient sample, compared to a reference sample.

In a twentieth aspect, the invention features a method of treatment or prophylaxis for a patient that has a tumor or is at risk for developing a tumor, comprising vaccinating the patient with a tumor antigen encoded by a nucleic acid selected from: TRAAM; TPR/UBP3; UBP3; BRAP-2/H⁺-ATPase; H⁺-ATPase; KOO8-1; MAIAP; Gene AS; BR-1; BR-2; KIAA0603; TPR; NOR-90; and BRAP-2.

In preferred embodiments of the twentieth aspect of the invention, the vaccinating may be with a tumor antigen polypeptide, or with a nucleic acid encoding the tumor antigen polypeptide, and the nucleic acid may be within an expression vector. In another embodiment of the twentieth aspect, the nucleic acid is within a cell capable of expressing the nucleic acid. The nucleic acid may be introduced into the cell in vivo or ex vivo.

In a twenty-first aspect, the invention features a method for treating a tumor in a patient, comprising administering to the patient, an antibody that specifically binds a tumor antigen encoded by a nucleic acid selected from TRAAM; TPR/UBP3; UBP3; BRAP-2/H⁺-ATPase; H⁺-ATPase; KOO8-1; MAIAP; Gene AS; BR-1; BR-2; KIAA0603; TPR; NOR-90; and BRAP-2. In a preferred embodiment, the antibody is coupled to a toxic or radioactive moiety.

In a twenty-second aspect, the invention features a method for treating a tumor in a patient, comprising administering to the patient, cytotoxic T lymphocytes that specifically bind a tumor antigen encoded by a nucleic acid selected from TRAAM; TPR/UBP3; UBP3; BRAP-2/H⁺-ATPase; H⁺-ATPase; KOO8-1; MAIAP; Gene AS; BR-1; BR-2; KIAA0603; TPR; NOR-90; and BRAP-2.

In a twenty-third aspect, the invention features a method for detecting a tumor in a patient, comprising: a) introducing, into the patient, an antibody coupled to an imaging compound, wherein the antibody specifically binds a tumor antigen encoded by a nucleic acid selected from TRAAM; TPR/UBP3; UBP3; BRAP-2/H⁺-ATPase; H⁺-ATPase; KOO8-1; MAIAP; Gene AS; BR-1; BR-2; KIAA0603; TPR; NOR-90; and BRAP-2, and b) detecting immune complexes formed between the antibody and the tumor antigen in the patient.

In a twenty-fourth aspect, the invention features a vaccine for treatment of a tumor or prophylaxis against developing a tumor, the vaccine comprising a tumor antigen polypeptide or a fragment thereof, wherein the tumor antigen polypeptide comprises a polypeptide substantially identical to a polypeptide encoded by a nucleic acid selected from TRAAM; TPR/UBP3; UBP3; BRAP-2/H⁺-ATPase; H⁺-ATPase; KOO8-1; MAIAP; Gene AS; BR-1; BR-2; KIAA0603; TPR; NOR-90; and BRAP-2.

In a twenty-fifth aspect, the invention features a vaccine for treatment of a tumor or prophylaxis against developing a tumor, the vaccine comprising a nucleic acid encoding a tumor antigen, or a fragment thereof, wherein said tumor antigen is substantially identical to a tumor antigen encoded by a nucleic acidselected from TRAAM; TPR/UBP3; UBP3; BRAP-2/H-ATPase; H⁺-ATPase; KOO8-1; MAIAP; Gene AS; BR-1; BR-2; KIAA0603; TPR; NOR-90; and BRAP-2.

In preferred embodiments of the twenty-fifth aspect, the nucleic acid may be within a cell capable of expressing the nucleic acid, and the nucleic acid may be within a vector.

In a preferred embodiment of the first through fifth, fifteenth through seventeenth, and twenty through twenty-fifth aspects of the invention, the tumor is metastatic.

In a twenty-sixth aspect, the invention features a method of identifying a nucleic acid encoding a tumor antigen or a fragment thereof, comprising: a) identifying a patient with a tumor; b) vaccinating the patient with a vaccine preparation comprising allogeneic tumor cells together with a GM-CSF sustained delivery system to generate an immune response in the patient; and c) isolating, either from an autologous post-vaccination tumor sample obtained from the patient, or from allogeneic tumor cells, nucleic acid that encodes a tumor antigen or a fragment thereof, wherein the nucleic acid encoding the tumor antigen or fragment is detected by an antibody in serum obtained from the patient, wherein the antibody specifically binds the tumor antigen.

In a twenty-seventh aspect, the invention features a method of identifying a nucleic acid encoding a tumor antigen or a fragment thereof, comprising: a) identifying a patient with a tumor;

b) vaccinating the patient with a vaccine preparation comprising allogeneic tumor cells together with a GM-CSF sustained delivery system to generate an immune response in the patient; and c) isolating, either from an autologous post-vaccination tumor sample obtained from the patient, or from allogeneic tumor cells, nucleic acid that encodes a tumor antigen or tumor antigen fragment, wherein the nucleic acid encoding the tumor antigen or fragment is detected by a cytotoxic T lymphocyte obtained from the patient, wherein the cytotoxic T lymphocyte specifically binds the tumor antigen.

In a twenty-eighth aspect, the invention features a method of identifying a tumor antigen or a fragment thereof, comprising: a) identifying a patient with a tumor; b) vaccinating the patient with a vaccine preparation comprising allogeneic tumor cells together with a GM-CSF sustained delivery system to generate an immune response in the patient; and c) isolating the tumor antigen or tumor antigen fragment either from an autologous post-vaccination tumor sample obtained from the patient, or from allogeneic tumor cells, wherein the tumor antigen or tumor antigen fragment is detected by an antibody in serum obtained from the patient, wherein the antibody specifically binds the tumor antigen.

In a twenty-ninth aspect, the invention features a method of identifying a tumor antigen or a fragment thereof, comprising: a) identifying a patient with a tumor; b) vaccinating the patient with a vaccine preparation comprising allogeneic tumor cells together with a GM-CSF sustained delivery system to generate an immune response in the patient; and c) isolating the tumor antigen or tumor antigen fragment either from an autologous post-vaccination tumor sample obtained from the patient, or from allogeneic tumor cells, wherein the nucleic acid encoding the tumor antigen or tumor antigen fragment is detected by a cytotoxic T lymphocyte obtained from the patient, wherein the cytotoxic T lymphocyte specifically binds the tumor antigen.

In preferred embodiments of the twenty-sixth through twenty-ninth aspects of the invention, the GM-CSF sustained delivery system comprises a plasmid or viral expression vector encoding GM-CSF that is transfected or transduced into the allogeneic tumor cells prior to vaccination, or the GM-CSF sustained delivery system comprises cells expressing GM-CSF that are mixed with the allogeneic tumor cells prior to vaccination, or the GM-CSF sustained delivery system comprises microspheres releasing GM-CSF that are mixed with the allogeneic tumor cells prior to vaccination.

In other embodiments of the twenty-sixth through twenty-ninth aspects of the invention, the serum may be pre-vaccination serum, or post-vaccination serum, and detection by post-vaccination serum may be more sensitive than detection by pre-vaccination serum.

In still other embodiments of the twenty-sixth through twenty-ninth aspects of the invention, the cells are irradiated prior to vaccination, the allogeneic tumor cells may originate from a leukemia, a lymphoma, a brain tumor (e.g., a glioblastoma or a neuroblastoma), a melanoma, a sarcoma, or a carcinoma such as a uterine, cervical, testicular, liver, ovarian, lung (e.g., non-small cell lung), renal cell, colon, breast, prostate, or bladder carcinoma, and vaccination increases the number of T lymphocytes and/or plasma cells in the patient's tumor, relative to the number of T lymphocytes and/or plasma cells in the patient's tumor prior to the vaccination.

In a thirtieth aspect, the invention features a substantially pure MAIAP polypeptide, wherein the polypeptide includes an amino acid sequence substantially identical to the amino acid sequence set forth in SEQ ID NO: 12. In a preferred embodiment of the thirtieth aspect of the invention, the MAIAP polypeptide includes the amino acid sequence set forth in SEQ ID NO: 12.

In a thirty-first aspect, the invention features a substantially pure MAIAP nucleic acid, wherein the nucleic acid encodes the MAIAP polypeptide set forth in SEQ ID NO: 12. In a preferred embodiment of the thirty-first aspect of the invention, the nucleic acid includes the nucleotide sequence set forth in SEQ ID NO: 11.

In a thirty-second aspect, the invention features a substantially pure nucleic acid that includes at least 14 consecutive nucleotides that display at least 85%, 90%, 92%, 95%, or 98% sequence identity to a nucleotide sequence that is complementary to a nucleic acid that encodes MAIAP. In preferred embodiments of the thirty-second aspect of the invention, the nucleic acid includes at least 16, 18, 22, 25, 50, 75, or 100 consecutive nucleotides that display at least 85%, 90%, 92%, 95%, or 98% sequence identity to a nucleotide sequence that is complementary to a nucleic acid that encodes MAIAP, and the nucleic acid hybridizes under high stringency conditions to a MAIAP nucleic acid.

In a thirty-third aspect, the invention features a substantially pure nucleic acid including at least 14 nucleotides, wherein the nucleic acid hybridizes under high stringency conditions to a nucleic acid that encodes MAIAP. In a preferred embodiment of the thirty-third aspect of the invention, the nucleic acid includes at least 16, 18, 22, 25, 50, 75, or 100 nucleotides.

In further embodiments of the thirty-second and thirty-third aspects of the invention, the substantially pure nucleic acid is an antisense nucleic acid.

In a thirty-fourth aspect, the invention features a method for stimulating apoptosis in a population of cells. The method includes introducing into the cells a MAIAP antisense nucleic acid, wherein the MAIAP antisense nucleic acid decreases the level of MAIAP in the cells, wherein the decrease stimulates apoptosis in the population of cells.

In various preferred embodiments of the thirty-fourth aspect of the invention, the cells are tumor cells, the cells are exposed to an apoptotic stimulus before or after the MAIAP antisense nucleic acid is introduced into the cells, and the apoptotic stimulus is gamma irradiation or a chemotherapeutic agent.

In a thirty-fifth aspect, the invention features a method for inhibiting apoptosis in a population of cells having an increased risk for undergoing apoptosis. The method includes introducing into the cells a substantially pure MAIAP polypeptide, wherein the substantially pure MAIAP polypeptide inhibits apoptosis in the cells, compared to cells not containing the substantially pure MAIAP polypeptide.

In one preferred embodiment of the thirty-fifth aspect of the invention, the MAIAP polypeptide is encoded by a substantially pure MAIAP nucleic acid, wherein the nucleic acid is introduced into the cells. The MAIAP nucleic acid may be introduced into the cells ex vivo or in vivo.

In another preferred embodiment of the thirty-fifth aspect of the invention, the increased risk for undergoing apoptosis is caused by: exposure to gamma irradiation, exposure to a chemotherapeutic agent, exposure to a toxin, exposure to hypoxia, an injury, a degenerative disease, or an attack by cells of the immune system.

In a thirty-sixth aspect, the invention features a method of identifying a compound that modulates apoptosis or radiation sensitivity. The method includes the steps of: (a) exposing a sample to a test compound, wherein the sample comprises a MAIAP nucleic acid, a MAIAP reporter gene, or a MAIAP polypeptide; and (b) assaying for a change in the level of MAIAP biological activity in the sample, relative to a sample not exposed to the test compound, wherein an increase in the level of said MAIAP biological activity in the sample, relative to a sample not exposed to the compound, indicates a compound that inhibits apoptosis or decreases radiation sensitivity, and a decrease in the level of the MAIAP biological activity in the sample, relative to a sample not exposed to the compound, indicates a compound that stimulates apoptosis or increases radiation sensitivity.

In various preferred embodiments of the thirty-sixth aspect of the invention, the MAIAP nucleic acid is genomic DNA, cDNA, mRNA, cRNA, or a substantially pure genomic DNA fragment. In other preferred embodiments of the thirty-sixth aspect of the invention, the MAIAP nucleic acid, MAIAP reporter gene, or MAIAP polypeptide is within a cell, wherein the cell is exposed to the test compound.

In a thirty-seventh aspect, the invention features a substantially pure TRAAM polypeptide or a fragment thereof, wherein the fragment includes at least 10 amino acids, wherein the polypeptide includes an amino acid sequence substantially identical to the amino acid sequence set forth in SEQ ID NO: 18, or SEQ ID NO: 19.

In preferred embodiments of the thirty-seventh aspect of the invention, the TRAAM polypeptide or fragment includes the partial TRAM repeat sequence set forth in SEQ ID NO: 25, the full TRAM repeat sequence set forth in SEQ ID NO: 24, or the PSET repeat sequence set forth in SEQ ID NO: 30.

In a thirty-eighth aspect, the invention features a substantially pure TRAAM nucleic acid, wherein the nucleic acid encodes a TRAAM polypeptide substantially identical to the polypeptide set forth in SEQ ID NO: 18 or SEQ ID NO: 19. In a preferred embodiment of the thirty-eighth aspect of the invention, the TRAAM nucleic acid includes the nucleotide sequence set forth in SEQ ID NO: 17.

In a thirty-ninth aspect, the invention features a substantially pure nucleic acid that includes at least 14 consecutive nucleotides that display at least 85%, 90%, 92%, 95%, or 98% sequence identity to a nucleotide sequence that is complementary to a nucleic acid that encodes a TRAAM polypeptide (SEQ ID NO: 18 or 19). In preferred embodiments of the thirty-ninth aspect of the invention, the substantially pure nucleic acid includes 16, 18, 22, 25, 50, 75, or 100 consecutive nucleotides that display at least 85%, 90%, 92%, 95%, or 98% sequence identity to a nucleotide sequence that is complementary to a nucleic acid that encodes a TRAAM polypeptide (SEQ ID NO: 18 or 19), and the nucleic acid hybridizes under high stringency conditions to a TRAAM nucleic acid.

In a fortieth aspect, the invention features a substantially pure nucleic acid including at least 14 nucleotides, wherein the nucleic acid hybridizes under high stringency conditions to a nucleic acid that encodes a TRAAM polypeptide (SEQ ID NO: 18 or 19). In a preferred embodiment of the fortieth aspect of the invention, the substantially pure nucleic acid includes at least 16, 18, 22, 25, 50, 75, or 100 nucleotides.

In preferred embodiments of the thirty-ninth and fortieth aspects of the invention, the substantially pure nucleic acid is an antisense nucleic acid.

By “tumor antigen” is meant an immunogenic polypeptide expressed by tumor cells, or a nucleic acid that encodes a such a polypeptide. A tumor antigen may be broadly expressed in various types of normal and tumor cells, expressed only in tumor cells of a particular type (e.g., melanoma), or expressed only in some tumor cells of a particular type (e.g., in the melanoma cells of a first patient, but not in the melanoma cells second patient). Tumor antigens or nucleic acids that encode tumor antigens may be present at higher levels in patient samples than in reference samples. Moreover, antibodies against tumor antigens may be present in patient serum, saliva, or tears at higher levels than those found in reference samples. Patients that mount a sufficient immune response against a tumor antigen expressed by their tumor cells experience tumor regression. Examples of tumor antigens are described below. They include: TRAAM; TPR/UBP3; UBP3; BRAP-2/H⁺-ATPase; H⁺-ATPase; KOO8-1; MAIAP; Gene AS; BR-1; BR-2; KIAA0603; TPR; NOR-90; and BRAP-2 (see FIGS. 6 through 8 and 17; SEQ ID NOs: 1-19 and 45-48).

By “tumor antigen nucleic acid” is meant DNA or RNA that encodes a tumor antigen of the invention. The tumor antigen nucleic acid may also be complementary to the coding strand of a tumor antigen nucleic acid; hence, this definition includes primers, probes, and antisense nucleic acids.

By “vaccination” is meant administration of an immunogenic preparation comprising either tumor cells, tumor antigen, nucleic acid that encodes tumor antigen, cells expressing tumor antigen, or a mixture thereof, to a patient who has a tumor or is likely to develop a tumor. A vaccination may employ only one tumor antigen, or more than one tumor antigen. The vaccination stimulates an immune response within the patient, which may result in partial or complete inhibition of tumor growth or partial or complete tumor regression, if the patient's tumor bears a tumor antigen similar to a tumor antigen used for vaccination. In addition, vaccination may provide prophylaxis against the development of a new tumor that bears a tumor antigen similar to a tumor antigen used for vaccination.

By “GM-CSF sustained delivery system” is meant a means for ensuring that GM-CSF is released from a vaccination site for at least 24 hours after vaccination, such that the GM-CSF secretion rate is approximately 84 to 965 ng/106 GM-CSF-secreting cells/24 hours, or, sufficient to induce an immune response. A GM-CSF sustained delivery system may employ an expression vector encoding GM-CSF, which is transfected or transduced into autologous tumor cells obtained from the patient prior to using the cells for vaccination; hence, the transfected autologous tumor cells used for vaccination secrete GM-CSF. Alternatively, a GM-CSF sustained delivery system may employ an expression vector transfected or transduced into non-autologous cells, such as allogeneic tumor cells, or other cultured cells, which would, as a result, secrete GM-CSF. A GM-CSF sustained delivery system may encompass any cells that secrete sufficient GM-CSF as defined below. GM-CSF-secreting cells may be mixed with the autologous tumor cells, and the cell mixture is then used for vaccination. In addition, allogeneic tumor cells that secrete GM-CSF may be used alone for vaccination. A GM-CSF sustained delivery system may also employ microspheres that slowly release GM-CSF, such as those that may be obtained from Immunex or Novartis. The microspheres are mixed with autologous tumor cells or allogeneic tumor cells, and the tumor cell/microsphere mixture is used for vaccination. For vaccinations containing autologous or allogeneic tumor cells mixed with either GM-CSF-secreting cells or microspheres, sufficient GM-CSF-secreting cells or microspheres must be mixed with the tumor cells such that GM-CSF equivalent to that released by GM-CSF-secreting autologous tumor cells (e.g., 84 to 965 ng/106 GM-CSF-secreting autologous tumor cells/24 hours), sufficient to obtain an immune response to the vaccination, is released. Similar approaches may also be used to achieve a GM-CSF sustained delivery system that is combined in a vaccine preparation either with autologous tumor cells, allogenic tumor cells, or with one or more tumor antigens.

By “pre-vaccination serum” is meant serum derived from the blood of an individual who has not been vaccinated with tumor cells or a tumor antigen.

By “post-vaccination serum” is meant serum derived from the blood of an individual after the individual has been vaccinated with tumor cells or a tumor antigen and has mounted an immune response against the vaccination material.

By “treatment” or “amelioration” of a tumor is meant that a therapy (e.g., chemotherapy, radiation therapy, or vaccination with a tumor antigen in order to enhance an anti-tumor immune response), administered either alone or in combination with other therapies, alleviates disease in at least some patients to which the therapy is administered. For example, treatment of a patient's cancer might reduce or inhibit tumor growth, or might even induce partial or complete tumor regression. Furthermore, the treatment may be prophylactic in that it prevents the development of new tumors in a patient in remission from cancer or in a patient who has metastatic cancer.

By “prophylaxis” against a tumor is meant that protective therapy (such as vaccination with one or more tumor antigens) is administered to a subject adjudged to have a higher than average risk of developing a tumor. Subjects with a relatively high risk of developing a tumor include those having a family history of cancer, those having one or more genetic mutations that are associated with a high risk for cancer (e.g., a mutation that inactivates a tumor suppressor gene), those expressing relatively high levels of tumor antigen or antibodies against tumor antigen, and those who have cancer or are in remission from cancer.

By “sample” is meant a tumor or tissue biopsy, a lymph node, bone marrow, cells, blood, serum, urine, stool, sputum, saliva, or other specimen obtained from a patient. The sample is analyzed in order to determine the level of one or more tumor antigens, or the level of antibodies or cytotoxic T lymphocytes that specifically bind a tumor antigen, by methods that are known in the art. For example, ELISA is used to measure levels of tumor antigen or antibodies against tumor antigen, and the polymerase chain reaction is used to measure levels of tumor antigen nucleic acid.

By “reference sample” is meant a sample in which one or more tumor antigens or antibodies or cytotoxic T lymphocytes that specifically bind a tumor antigen have been measured, and to which levels of tumor antigen or antibodies or cytotoxic T lymphocytes that specifically bind a tumor antigen in a patient sample are compared. Reference levels may be higher, lower, or the same as patient sample levels. Comparison of patient sample levels and reference sample levels allows a diagnosis of cancer and/or a prognosis of a cancer, for patients whose cancer cells express the tumor antigen being measured.

By “high stringency conditions” is meant conditions that allow hybridization comparable with the hybridization that occurs using a DNA probe of at least 500 nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. (these are typical conditions for high stringency Northern or Southern hybridizations). High stringency hybridization is relied upon for the success of numerous techniques routinely performed by molecular biologists, such as high stringency PCR, DNA sequencing, single strand conformational polymorphism analysis, and in situ hybridization. In contrast to Northern and Southern hybridizations, these techniques are usually performed with relatively short probes (e.g., usually 16 nucleotides or longer for PCR or sequencing, and 40 nucleotides or longer for in situ hybridization). The high stringency conditions used in these techniques are well known to those skilled in the art of molecular biology, and may be found, for example, in F. Ausubel et al., Current Protocols in Molecular Biology, John Wiley & Sons, New York, N.Y., 1997, hereby incorporated by reference.

By “probe” or “primer” is meant a single-stranded DNA or RNA molecule of defined sequence that can base-pair to a second DNA or RNA molecule that contains a complementary sequence (the “target”). The stability of the resulting hybrid depends upon the extent of the base-pairing that occurs. The extent of base-pairing is affected by parameters such as the degree of complementarity between the probe and target molecules, and the degree of stringency of the hybridization conditions. The degree of hybridization stringency is affected by parameters such as temperature, salt concentration, and the concentration of organic molecules such as formamide, and is determined by methods known to one skilled in the art. Probes or primers specific for nucleic acid encoding a tumor antigen preferably have at least 35% sequence identity, more preferably at least 45-55% sequence identity, even more preferably at least 60-75% sequence identity, still more preferably at least 80-90% sequence identity, and most preferably 100% sequence identity. Probes may be detectably-labelled, either radioactively, or non-radioactively, by methods well-known to those skilled in the art. Probes are used for methods involving nucleic acid hybridization, such as: nucleic acid sequencing, nucleic acid amplification by the polymerase chain reaction, single stranded conformational polymorphism (SSCP) analysis, restriction fragment polymorphism (RFLP) analysis, Southern hybridization, Northern hybridization, in situ hybridization, electrophoretic mobility shift assay (EMSA).

By “pharmaceutically acceptable carrier” means a carrier that is physiologically acceptable to the treated mammal while retaining the therapeutic properties of the compound with which it is administered. One exemplary pharmaceutically acceptable carrier is physiological saline. Other physiologically acceptable carriers and their formulations are known to one skilled in the art and described, for example, in Remington's Pharmaceutical Sciences (18^{th} edition), ed. A. Gennaro, 1990, Mack Publishing Company, Easton, Pa.

By “substantially identical” is meant a polypeptide or nucleic acid exhibiting at least 80%, preferably at least 85%, more preferably at least 90%, and most preferably at least 95% identity to a reference amino acid or nucleic acid sequence. For polypeptides, the length of comparison sequences is at least 16 amino acids, preferably at least 20 amino acids, more preferably at least 25 amino acids, and most preferably 35 amino acids. For nucleic acids, the length of comparison sequences is at least 50 nucleotides, preferably at least 60 nucleotides, more preferably at least 75 nucleotides, and most preferably at least 110 nucleotides.

Sequence identity is typically measured using sequence analysis software with the default parameters specified therein (e.g., Sequence Analysis Software Package of the Genetics Computer Group, University of Wisconsin Biotechnology Center, 1710 University Avenue, Madison, Wis. 53705).

By “substantially pure polypeptide” is meant a polypeptide (or a fragment thereof) that has been separated from the components that naturally accompany it. Typically, the polypeptide is substantially pure when it is at least 60%, by weight, free from the proteins and naturally-occurring organic molecules with which it is naturally associated. Preferably, the polypeptide is a tumor antigen polypeptide that is at least 75%, more preferably at least 90%, and most preferably at least 99%, by weight, pure. A substantially pure tumor antigen polypeptide may be obtained, for example, by extraction from a natural source (e.g., a tumor cell), by expression of a recombinant nucleic acid encoding a tumor antigen polypeptide, or by chemically synthesizing the polypeptide. Purity can be measured by any appropriate method, e.g., by column chromatography, polyacrylamide gel electrophoresis, or HPLC analysis.

A protein is substantially free of naturally associated components when it is separated from those contaminants which accompany it in its natural state. Thus, a protein which is chemically synthesized or produced in a cellular system different from the cell from which it naturally originates will be substantially free from its naturally associated components. Accordingly, substantially pure polypeptides not only includes those derived from eukaryotic organisms but also those synthesized in E. coli or other prokaryotes.

By “substantially pure DNA” is meant DNA that is free of the genes which, in the naturally-occurring genome of the organism from which the DNA of the invention is derived, flank the gene. The term therefore includes, for example, a recombinant DNA which is incorporated into a vector; into an autonomously replicating plasmid or virus; or into the genomic DNA of a prokaryote or eukaryote; or which exists as a separate molecule (e.g., a cDNA or a genomic or cDNA fragment produced by PCR or restriction endonuclease digestion) independent of other sequences. It also includes a recombinant DNA which is part of a hybrid gene encoding additional polypeptide sequence.

By “transformation” is meant any method for introducing foreign molecules into a cell, e.g., a bacterial, yeast, fungal, algal, plant, insect, or animal cell. Lipofection, DEAE-dextran-mediated transfection, microinjection, protoplast fusion, calcium phosphate precipitation, transduction (e.g., bacteriophage, adenoviral or retroviral delivery), electroporation, and biolistic transformation are just a few of the methods known to those skilled in the art which may be used.

By “transformed cell,” “transfected cell,” or “transduced cell,” means a cell (or a descendent of a cell) into which a DNA molecule encoding a polypeptide of the invention has been introduced, by means of recombinant DNA techniques.

By “promoter” is meant a minimal sequence sufficient to direct transcription. Also included in the invention are those promoter elements which are sufficient to render promoter-dependent gene expression controllable for cell type-specific, tissue-specific, temporal-specific, or inducible by external signals or agents; such elements may be located in the 5′ or 3′ or intron sequence regions of the native gene.

By “operably linked” is meant that a gene and one or more regulatory sequences are connected in such a way as to permit gene expression when the appropriate molecules (e.g., transcriptional activator proteins) are bound to the regulatory sequences.

By “detectably-labeled” is meant any means for marking and identifying the presence of a molecule, e.g., an oligonucleotide probe or primer, a gene or fragment thereof, a cDNA molecule, or an antibody. Methods for detectably-labeling a molecule are well known in the art and include, without limitation, radioactive labeling (e.g., with an isotope such as ³²P or ³⁵S) and nonradioactive labeling (e.g., chemiluminescent labeling, e.g., fluorescein labeling).

By “purified antibody” is meant antibody which is at least 60%, by weight, free from proteins and naturally occurring organic molecules with which it is naturally associated. Preferably, the preparation is at least 75%, more preferably 90%, and most preferably at least 99%, by weight, antibody, e.g., an antibody that binds a polypeptide or fragment of a polypeptide disclosed herein. A purified antibody may be obtained, for example, by affinity chromatography using recombinantly-produced protein or conserved motif peptides and standard techniques.

By “specifically binds” is meant an antibody that recognizes and binds a given tumor antigen polypeptide but that does not substantially recognize and bind other molecules in a sample, e.g., a biological sample, that naturally includes protein.

By “antisense nucleic acid” is meant a nucleic acid complementary to (i.e., that base-pairs with) a tumor antigen-encoding nucleic acid of the invention, e.g., a MAIAP nucleic acid. Preferably, the antisense nucleic acid decreases expression (i.e., transcription and/or translation) of the tumor antigen-encoding nucleic acid (e.g., MAIAP) by at least 5%, more preferably by at least 10%, still more preferably by at least 20% to 30%, and most preferably by at least 50% to 70%.

By “MAIAP polypeptide” is meant a polypeptide comprising an amino acid sequence that is substantially identical, as defined above, to the MAIAP polypeptide sequence shown in FIG. 17 and set forth in SEQ ID NO: 12.

By “MAIAP biological activity” is meant the ability of a MAIAP polypeptide to inhibit apoptosis or decrease radiation sensitivity in cells containing the MAIAP polypeptide, relative to cells not containing the MAIAP polypeptide. The level of MAIAP biological activity may be directly measured using any of the many known assays for measuring apoptosis or assessing relative radiation sensitivity. The relative level of MAIAP biological activity may also be assessed by measuring the level of MAIAP mRNA (e.g., by reverse transcription-polymerase chain reaction (RT-PCR) amplification or Northern hybridization), the level of MAIAP protein (e.g., by ELISA or Western hybridization), the activity of a reporter gene under the transcriptional regulation of a MAIAP transcriptional regulatory region (by reporter gene assay, as described below), or the specific interaction of MAIAP with another molecule (e.g., by the two-hybrid assay).

By “apoptosis” is meant a cell death pathway wherein a dying cell displays a set of well-characterized biochemical hallmarks that include cytolemmal membrane blebbing, cell soma shrinkage, chromatin condensation, nuclear disintegration, and DNA laddering. There are many well-known assays for determining the apoptotic state of a cell, including, and not limited to: reduction of MTT tetrazolium dye, TUNEL staining, Annexin V staining, propidium iodide staining, DNA laddering, PARP cleavage, caspase activation, and assessment of cellular and nuclear morphology. Any of these or other known assays may be used in the methods of the invention to determine whether cells are undergoing apoptosis.

By “increased risk for undergoing apoptosis” is meant a population of cells that is exposed to an apoptotic stimulus, e.g., gamma irradiation, a chemotherapeutic agent, a toxin, an injury, an attack by cells of the immune system, hypoxia, or a degenerative disease. Apoptosis is stimulated in such a cell population by at least 5%, preferably by at least 10%, more preferably by at least 25%, still more preferably by at least 50%, and most preferably by at least 75%.

By “stimulating apoptosis” is meant increasing the number of apoptotic cells in a population of cells by at least 5%, preferably by at least 10%, more preferably by at least 25%, still more preferably by at least 50%, and most preferably by at least 75%.

By “inhibiting apoptosis” is meant decreasing the number of apoptotic cells in a population of cells by at least 1% to 5%, preferably by at least 10%, more preferably by at least 25%, still more preferably by at least 50%, and most preferably by at least 75%.

By “test compound” is meant a chemical, be it naturally-occurring or artificially-derived, that is surveyed for its ability, when applied to cells, cell lysates, or fractions thereof, to modulate radiation sensitivity, susceptibility for undergoing apoptosis, or tumor antigen (e.g., MAIAP) biological activity, in one of the assay methods described herein. Test compounds may include, for example, peptides, polypeptides, synthesized organic molecules, naturally occurring organic molecules, and nucleic acid molecules.

By “expose” is meant to allow contact between an animal, cell, lysate or extract derived from a cell, or molecule derived from a cell, and a test compound or apoptotic stimulus (e.g., radiation or a chemotherapeutic agent).

By “assaying” is meant analyzing the effect of a treatment, be it chemical or physical, administered to whole animals, cells, or molecules derived therefrom. The material being analyzed may be an animal, a cell, a lysate or extract derived from a cell, or a molecule derived from a cell. The analysis may be, for example, for the purpose of detecting altered gene expression, altered RNA stability, altered protein stability, altered protein levels, or altered protein biological activity. The means for analyzing may include, for example, antibody labeling, immunoprecipitation, phosphorylation assays, and methods known to those skilled in the art for detecting nucleic acids.

By “sample” is meant an animal, a cell, a lysate or extract derived from a cell, or a molecule derived from a cell or cellular material, which is assayed as described above.

By “modulating” is meant changing, either by decrease or increase.

By “a decrease” is meant a lowering in the level of: a) protein (e.g., as measured by ELISA); b) reporter gene activity (e.g., as measured by reporter gene assay, for example, lacZ/β-galactosidase, green fluorescent protein, or luciferase activity); c) mRNA (e.g., as measured by RT-PCR relative to an internal control, for example, a “housekeeping” gene product such as β-actin or glyceraldehyde 3-phosphate dehydrogenase (GAPDH)); or d) the number of apoptotic cells in a test sample. In all cases, the lowering is preferably by at least 30%, more preferably by at least 40% to 60%, and even more preferably by at least 70%.

By “an increase” is meant a rise in the level of: a) protein (e.g., as measured by ELISA); b) reporter gene activity (e.g., as measured by reporter gene assay, for example, lacZ/β-galactosidase, green fluorescent protein, or luciferase activity); c) mRNA (e.g., as measured by RT-PCR relative to an internal control, for example, a “housekeeping” gene product such as β-actin or glyceraldehyde 3-phosphate dehydrogenase (GAPDH)) or d) the number of apoptotic cells in a test sample. Preferably, the increase is by at least 1.5-fold to 2-fold, more preferably by at least 3-fold, and most preferably by at least 5-fold.

By “alteration in the level of gene expression” is meant a change transcription, translation, or mRNA or protein stability such that the overall amount of a product of the gene, i.e., mRNA or polypeptide, is increased or decreased.

By “reporter gene” is meant any gene that encodes a product whose expression is detectable and/or quantitatable by immunological, chemical, biochemical or biological assays. A reporter gene product may, for example, have one of the following attributes, without restriction: fluorescence (e.g., green fluorescent protein), enzymatic activity (e.g., lacZ/β-galactosidase, luciferase, chloramphenicol acetyltransferase), toxicity (e.g., ricin A), or an ability to be specifically bound by a second molecule (e.g., biotin or a detectably labelled antibody). It is understood that any engineered variants of reporter genes, which are readily available to one skilled in the art, are also included, without restriction, in the foregoing definition.

By “tumor antigen fragment” is meant a protein fragment comprising at least 10 amino acids, preferably at least 15, 20, 25, or 30 amino acids, and most preferably at least 50 amino acids, which correspond to an amino acid sequence of a tumor antigen as defined above.

### BRIEF DESCRIPTION OF THE DRAWINGS

FIG. 1 is a graph showing the relative reactivity of sixty normal serum samples against a MAIAP-GST fusion protein.

FIG. 2 is a graph showing that sera from 4 out of 48 vaccinated melanoma patients and 5 out of 15 vaccinated lung cancer patients show relatively high reactivity against MAIAP.

FIG. 3 is a graph showing that sera from 0 out of 15 prostate cancer patients show higher than average reactivity against MAIAP.

FIG. 4 is a graph showing a time course of the relative levels of anti-MAIAP antibodies in the serum of a melanoma patient (K030) before, during, and after vaccination with GM-CSF-secreting autologous tumor cells.

FIG. 5 is a diagram of cell cycle analyses showing that transfection of MAIAP into A293 cells increases their resistance to radiation.

FIG. 6 is a diagram showing the full-length sequence of the TRAAM_{U937} cDNA clone (SEQ ID NO: 17).

FIG. 7 is a diagram showing the long open reading frame of the TRAAM_{U937} cDNA clone (TRAAM_{U937}-ORF1; SEQ ID NO: 18), which contains the PSET peptide.

FIG. 8 is a diagram showing the short open reading frame of the TRAAM_{U937} cDNA clone (TRAAM_{U937}-ORF2; SEQ ID NO: 19), which contains the TRAM peptide.

FIG. 9 is a series of graphs showing the results of ELISA assays using anti-PSET antiserum or anti-TRAM antiserum and PSET, TRAM, and MUC-1 peptides as antigen.

FIG. 10 is a graph showing the results of ELISA assays using anti-MUC-1 antiserum and PSET, TRAM, and MUC-1 peptides as antigen.

FIG. 11(A-H) is a series of photomicrographs of tissues from patients before and after vaccination with irradiated GM-CSF-secreting melanoma cells.

FIG. 12 is a chart showing that vaccination with irradiated GM-CSF-secreting melanoma cells stimulates cytokine production by peripheral blood lymphocytes.

FIG. 13 is a graph demonstrating the potent cytotoxicity of tumor infiltrating lymphocytes from a vaccinated patient.

FIG. 14 is a chart showing cytokine production by tumor infiltrating lymphocytes from a vaccinated patient.

FIG. 15 is a Western blot demonstrating that vaccination with irradiated GM-CSF-secreting melanoma cells stimulates anti-melanoma antibody responses.

FIG. 16 is a chart that showing that vaccination with irradiated GM-CSF-secreting melanoma cells stimulates anti-melanoma antibody responses.

FIG. 17(A-K) is a set of DNA sequences that encode tumor antigens and tumor antigen polypeptide fragments of the invention, and their encoded polypeptides (SEQ ID NOs: 1-16).

### DETAILED DESCRIPTION OF THE INVENTION

The clinical study described herein demonstrates that vaccination with irradiated, autologous melanoma cells engineered to secrete granulocyte-macrophage colony stimulating factor (GM-CSF) consistently augments anti-tumor cellular and humoral immunity in patients with metastatic melanoma. The most convincing evidence that this immunization scheme (previously published in Soiffer et al., Hum. Gene Ther., 8:111-123, 1997) enhances anti-melanoma immunity is the finding that distant metastases were frequently infiltrated by large numbers of T lymphocytes and plasma cells following, but not before, vaccination. Anti-melanoma immune reactions were found in metastases, including bulky lesions, derived from a variety of sites, and were documented pathologically in one patient to be persistent five months after the completion of therapy in all eight sites of metastatic disease. Immunohistochemical analysis demonstrated that both CD4 and CD8 positive T cells were in direct contact with dying melanoma cells. Analysis of the infiltrating T lymphocytes and plasma cells suggested several potential anti-tumor effector mechanisms including lymphocyte-mediated cytotoxicity, cytokine production, and antibody formation. Anti-melanoma immune responses were more intense at dose levels 2 and 3 than at dose level 1 (dose levels are described below), though no clear relationship to the level of GM-CSF secretion could be delineated.

Histopathologic assessment revealed that the coordinated activation of T lymphocytes and plasma cells resulted in destruction of at least 80% of the tumor cells in the infiltrated metastases. In most cases, however, these anti-tumor immune responses failed to induce clinical regressions; rather, the necrotic tumor masses were largely replaced by inflammatory cells, edema, and extensive fibrosis. These findings underscore the limitations of relying exclusively upon traditional measurements of tumor shrinkage in assessing the anti-tumor activity of this vaccination scheme. Additional studies are required to clarify the mechanisms underlying the resistance of the residual tumor cells.

Other strategies to enhance the frequency and intensity of anti-melanoma immune responses are under clinical investigation. These include vaccination with autologous, hapten-modified tumor cells in conjunction with Bacille Calmette Guerin (BCG) and cyclophosphamide (Berd et al., Cancer Res., 51:2731-2734, 1991); immunization with allogeneic melanoma cells in a variety of forms, including intact cells or shed antigens with BCG, viral-modified cell lysates, and cell lysates admixed with complex adjuvants (Oratz et al., J. Biol. Resp. Modif., 8:355-358, 1989; Mitchell et al., J. Clin, Oncol., 8:856-869, 1990; Hersey et al., Cancer Immunol. Immunother, 25:257-265, 1987; Morton et al., Ann. Surg., 216:463-482, 1992; Singhuff et al., J. Surg. Oncol., 39:139-147); and vaccination with defined melanoma antigens such as the ganglioside GM2 or peptides derived from the MAGE and melanocyte differentiation protein families (Livingston et al., J. Clin. Oncol., 12: 1036-1044, 1994; Marchand et al., Int. J. Cancer, 63:883-885 1995; Jager et al., Int. J. Cancer, 67:54-62, 1996). However, the prominent plasma cell infiltration, IgG antibody response, extensive fibrosis, and vasculopathy observed in the work described herein have not been described with hapten-modified tumor cell vaccines. Further investigations are required to characterize these differences more thoroughly and to determine whether the mechanisms underlying the two vaccination strategies involve distinct or overlapping pathways.

Several additional features of the anti-tumor immune responses elicited here underscore distinctive properties of this immunization scheme. First, all patients developed impressive admixtures of dendritic cells, macrophages, eosinophils, and T lymphocytes at vaccination sites. The dramatic influx of dendritic cells and macrophages supports the hypothesis, derived from studies in experimental murine model systems, that GM-CSF functions to improve tumor antigen presentation by increasing the numbers and activities of host-derived antigen-presenting cells (Dranoff et al., Proc. Natl. Acad. Sci., U.S.A., 90:3539-3543, 1993). All patients also developed, as a function of vaccination, intense infiltrates of T lymphocytes and eosinophils in response to injections of irradiated, non-transfected melanoma cells. While the antigens stimulating these delayed-type hypersensitivity reactions remain to be determined (and in particular to delineate whether they represent previously reported or novel melanoma targets and/or components of the culture media), the prominent eosinophil component distinguishes these infiltrates from the classical tuberculin-type reactions generated by other vaccination schemes (Barth et al., Cancer. Res., 54:3342-3345, 1994). This histopathology, moreover, bears a remarkable resemblance to that observed in allergic disease and parasitic infection, suggesting intriguing connections among these forms of immunity (Hus et al., J. Allergy Clin. Immunol., 54:339-349, 1974).

Histopathologic examination of metastases resected following vaccination delineated the context in which eosinophils mediate anti-tumor activity. Degranulating eosinophils (along with neutrophils and lymphocytes) were found in association with damaged endothelium within the tumor vasculature; this vasculopathy resulted in significant zonal necrosis within the tumor mass.

Further clinical development of this cancer immunization strategy will require simpler methods of vaccine production that are more amenable to widespread application. For example, adenoviral vectors may be used to transfer genes to freshly dissociated, non-replicating tumor cells from patients who have, e.g., metastatic melanoma or non-small cell lung carcinoma.

Tumor Antigens

In order to identify individual tumor antigens involved in inducing anti-melanoma immunity, we established a melanoma cell line from a post-vaccination metastatic tumor that demonstrated striking T cell and plasma cell infiltration upon removal, and constructed a cDNA expression library from mRNA isolated from the melanoma cell line. The library was screened with post-vaccination serum, and the following clones were isolated: MAIAP (SEQ ID NOs: 11 and 12); TRAAM (SEQ ID NOs: 1-4; also SEQ ID NOs: 17-19); KIAA0603 (SEQ ID NOs: 45 and 46); TPR/UBP-3 (SEQ ID NO: 7); BRAP-2/H⁺-ATPase (SEQ ID NO: 8); KOO8-1 (SEQ ID NOs: 9 and 10); NOR-90 (SEQ ID NO: 13); BR-1 (SEQ ID NO: 14); BR-2 (SEQ ID NO: 15); and Gene AS (SEQ ID NO: 16).

1. MAIAP

MAIAP (melanoma-associated inhibitor of apoptosis protein; SEQ ID NOs: 11 and 12), which we originally designated “IAP-M,” is a novel member of the “inhibitor of apoptosis” protein family. There is a single EST match in the database (AA379765; SEQ ID NO: 49; from a human skin tumor).

Northern analysis demonstrated highest expression in spinal cord, with lower expression in testis, placenta, and lymph node. We observed transcripts of various sizes, including a major species of approximately 1.5 kilobases (kb) and minor species of about 4.0, 2.0, and 0.8 kb. MAIAP expression in a melanoma cell line derived from the patient whose sera was used to clone MAIAP was approximately 30- to 50-fold higher than in spinal cord. We have also observed expression in a second melanoma cell line (derived from a different patient in our clinical trial), two lung cancer cell line (A549 and H125), and a transformed kidney cell line (A293).

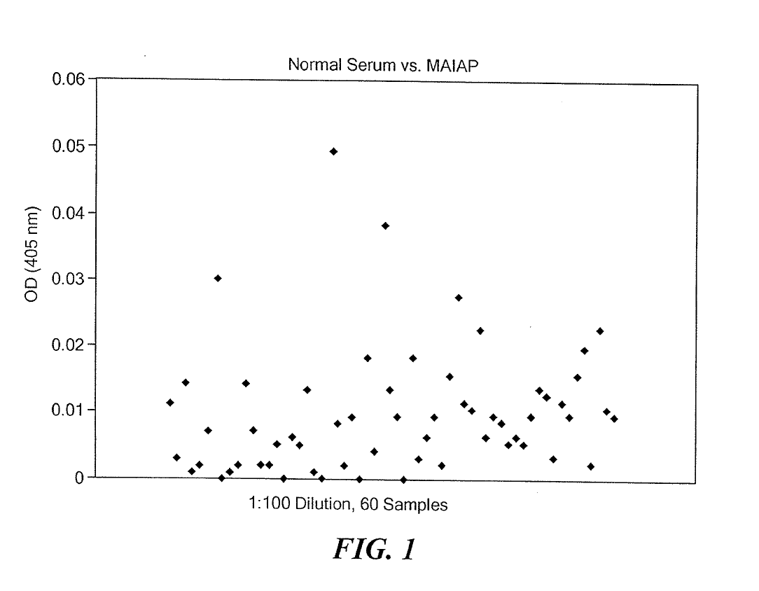

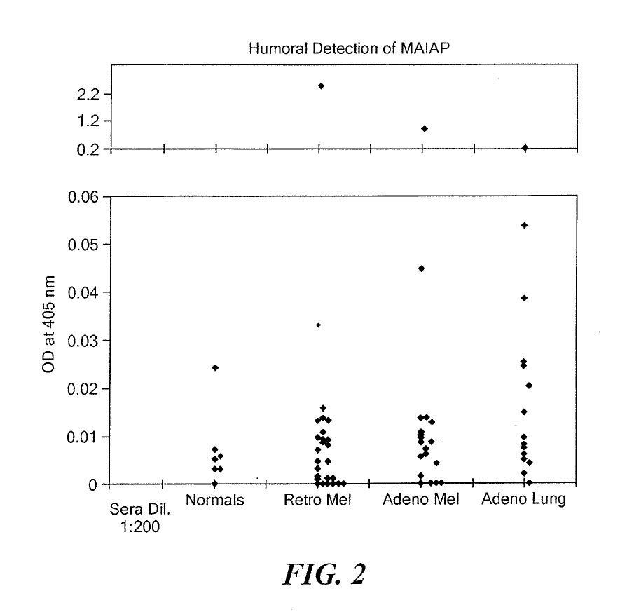

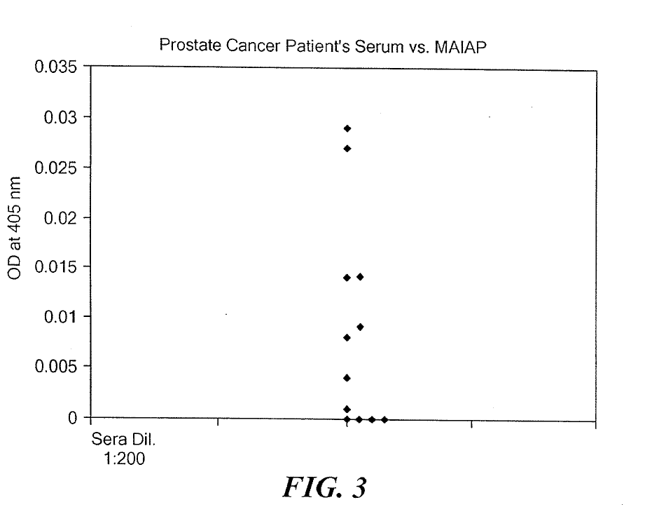

We have produced recombinant MAIAP in bacteria as a GST fusion protein. This protein product is recognized by Western analysis using patient sera. We have set up an ELISA assay to test reactivity of patient sera against MAIAP. Sixty normal blood bank donors (30 male and 30 female) showed minimal levels (FIG. 1) of IgG MAIAP-specific antibodies at either 1:100 or 1:200 sera dilutions. In contrast, when tested at sera dilutions of 1:200, 4 out of 48 melanoma patients (treated on one of two GM-CSF-secreting, autologous melanoma cell protocols; either a retroviral vector encoding GM-CSF or an adenoviral vector encoding GM-CSF were used to genetically modify the tumor cells in the respective trials) demonstrated significant reactivity against MAIAP (FIG. 2; “Retro MeI” and “Adeno MeI” respectively). Moreover, the sera of 5 out of 15 lung cancer patients (treated with a GM-CSF-secreting autologous tumor cell vaccine in a vaccine trial for lung cancer) demonstrated reactivity against MAIAP (FIG. 2; “Adeno Lung”). By contrast, 0 out of 15 prostate cancer patients tested to date showed reactivity against MAIAP (FIG. 3; compare with normal serum samples in FIG. 1).

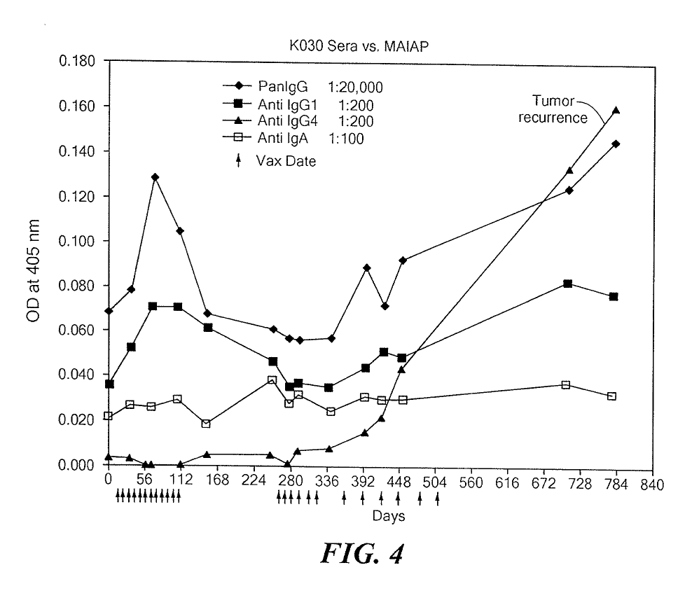

A detailed analysis of reactivity against MAIAP has been conducted in patient K030, who participated in the trial testing the efficacy of vaccination with autologous tumor cells expressing retrovirally-encoded GM-CSF. This patient demonstrated very high reactivity against MAIAP at the time of entering the study (i.e., prior to receiving the vaccination). Nonetheless, vaccination induced an increase in anti-MAIAP antibody titers (FIG. 4; the abscissa refers to the number of days after entering the study, and the arrows on the abscissa represent the dates on which the patient was vaccinated). The vaccination induced the development of IgG4 antibodies against MAIAP, which were not present upon the patient's entry into the study. In contrast to reactivity against MAIAP, reactivity against control antigens (from candida yeast and mumps virus) was not affected by vaccination. Significantly, the increased anti-MAIAP antibody titers were temporally associated with tumor destruction in this patient.

We identified two peptides derived from MAIAP which bind to HLA-A2 class I molecules. These are: JS34 (SLGSPVLGL; SEQ ID NO: 22) AND JS90 (RLASFYDWPL; SEQ ID NO: 23). They were identified using the peptide motif scoring system “HLA Peptide Binding Predictions” (K. C. Parker et al., J. Immunol., 152:163, 1994), which is available through the World Wide Web at: http://bimas.dcrt.nih.gov:80/cgi-bin/molbio/ken_parker_comboform. One of skill in the art will recognize that other MAIAP peptides that bind HLA molecules may be identified using analogous programs. We have produced HLA-A2 soluble tetramers folded with these peptides, and will use them to assess the presence of anti-MAIAP cytotoxic T lymphocytes in the blood of HLA-2-positive patients that show antibody reactivity to MAIAP.

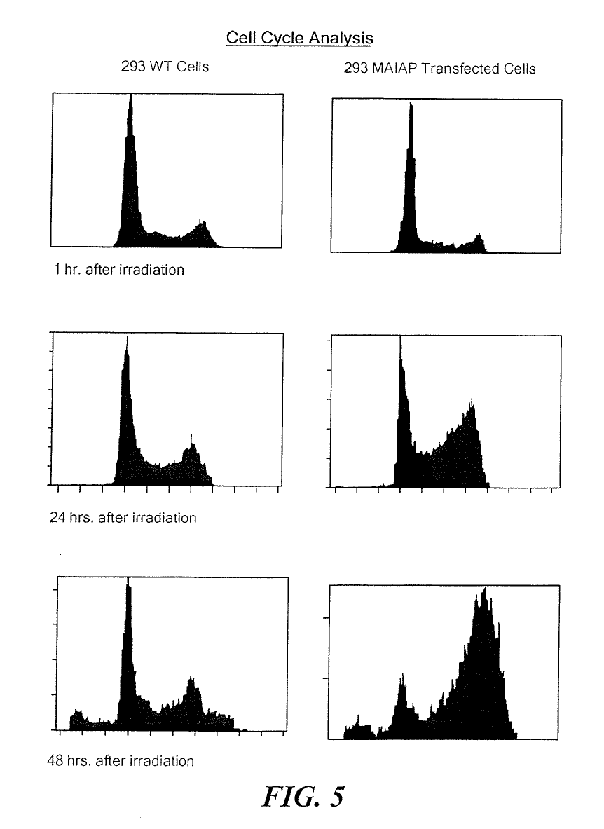

We have constructed a high-titer retroviral vector encoding MAIAP. This vector was used to transfect A293 cells in order to generate a partner cell line expressing high levels of MAIAP. Preliminary studies have shown that MAIAP-transfected cells demonstrate superior resistance to radiation, compared to untransfected A293 cells. Briefly, we irradiated untransfected and MAIAP-transfected A293 cells with 15,000 rads, harvested cells 1 hour, 24 hours, and 48 hours after irradiation, stained the harvested cells with propidium iodide, and subjected them to cell cycle analysis using a fluorescence-activated cell sorter (FACS). The cell cycle profiles shown in FIG. 5 indicate a markedly increased proportion of transfected cells accumulating in the G2/M phase, compared to untransfected cells. Accumulation in the G2/M phase allows for repair of radiation-induced DNA damage. This finding suggests that high-level expression of MAIAP is associated with the development of radiation-resistant cancer cells and that MAIAP increases cellular resistance to apoptotic stimuli. Therefore, methods for targeting cells that overexpress MAIAP, as well as methods for inhibiting MAIAP expression or MAIAP biological activity, may prove to be valuable anti-cancer strategies. Various anti-MAIAP strategies could be combined for maximum efficacy. For example, a drug that inhibits MAIAP activity, such as a MAIAP antisense nucleic acid, dominant-negative protein, or small-molecule inhibitor, could be used in combination with a vaccine containing tumor cells that naturally overexpress or are genetically engineered to overexpress MAIAP. Such combination therapies might be more effective than either therapy alone.

MAIAP Therapy for Inhibition of Apoptosis

Because MAIAP, a member of the IAP (inhibitor of apoptosis) family, is highly expressed in the spinal cord, it is possible that altered expression of MAIAP or mutations in the MAIAP gene may be associated with diseases that affect the spinal cord. For example, NAIP, another member of the IAP family that is highly expressed in the nervous system, has been shown to be mutated in spinal muscular atrophy, a neurodegenerative disease.

Given the relationship of MAIAP to other IAPs, it is likely that therapies for increasing the intracellular level of MAIAP in cells that are at increased risk for undergoing apoptosis will prove useful for treating diseases or conditions that involve higher-than-normal levels of cell death. Examples of cells and diseases, conditions, or situations in which it would be desirable to inhibit apoptosis include, but are not limited to: neurons (e.g., in degenerative and autoimmune diseases of the central or peripheral nervous system, such as stroke, Alzheimer's disease, Parkinson's disease, multiple sclerosis, and amyotrophic lateral sclerosis) cardiomyocytes (e.g., in heart disease or post-myocardial infarction), skeletal myocytes (e.g., in muscular degenerative disease, such as Duchenne's muscular dystrophy), kidney and liver cells (e.g., in early stages of progressive organ failure from disease or exposure to toxins), hair follicle cells (e.g., in hair loss), ovarian follicle cells, ova, sperm cells (e.g., in infertility), pancreatic islet cells, e.g., beta cells (e.g., in autoimmune diabetes) or retinal photoreceptor cells (e.g., in retinal degenerative conditions such as those resulting from retinitis pigmentosa, chemical toxicity, retinal detachment, glaucoma, diabetes, and axotomy).

Expression vectors encoding MAIAP may also be introduced into cells ex vivo in order to enhance the survival of cell or organ transplants. For example, the vectors may be introduced into pancreatic beta cells prior to transplantation into diabetic patients or into dopaminergic neurons prior to transplantation into Parkinson's patients. Transplanted cells containing MAIAP expression vectors are more likely to survive in the patient after transplantation than cells not containing such vectors.

MAIAP Antisense Therapy

We have shown that MAIAP, a member of the IAP family, increases cellular resistance to radiation exposure. Accordingly, decreasing the intracellular level of MAIAP polypeptide by antisense therapy should be a useful therapeutic approach for sensitizing tumor cells to apoptotic stimuli, such as gamma-radiation therapy and chemotherapeutic agents.

Antisense therapy is based on the well-known principle of suppressing gene expression by intracellular hybridization of endogenous nucleic acid (genomic DNA or mRNA) molecules encoding a protein of interest with a complementary antisense nucleic acid, such as an antisense oligonucleotide or antisense RNA. Antisense nucleic acids may inhibit protein expression at the transcriptional level, at the translational level, or at both levels. Antisense oligonucleotides or antisense RNA, generated by well-known methods, may be administered to patients by conventional drug delivery techniques. The antisense nucleic acids enter the appropriate cell type and hybridize with the endogenous target nucleic acid to inhibit transcription or translation of the target protein. Antisense mRNA may also be provided intracellularly to a patient by administration of a gene therapy vector encoding an antisense RNA of interest. Expression of the antisense RNA may limited to a particular cell type, for example, by placing a DNA molecule encoding the antisense RNA under the transcriptional regulation of a tissue-specific promoter. Inhibition of MAIAP transcription or translation using MAIAP antisense RNA decreases a cell's resistance to various apoptotic stimuli, e.g., exposure to radiation or toxins such as cancer chemotherapeutic agents.

Numerous examples of therapeutic benefit derived from antisense therapy are known in the art. Just a few representative examples are described in: Gokhale et al., Gene Ther. 4:1289-1299, 1997; Martens et al., Proc. Natl. Acad. Sci. USA 95:2664-2669, 1998; Offensperger et al., Mol. Biotechnol. 9:161-170, 1998; Kondo et al., Oncogene 16:3323-3330, 1998; and Higgens et al., Proc. Natl. Acad. Sci. USA 90:9901-9905, 1993.

MAIAP antisense nucleic acids contain at least 10 consecutive nucleotides that are complementary to (i.e., base-pair with) a MAIAP mRNA or DNA sequence, and preferably contain 14-18 consecutive nucleotides that are complementary to a MAIAP mRNA or DNA. MAIAP antisense nucleic acids may contain 25, 40, 60, 85, 120, or more consecutive nucleotides that are complementary to a mRNA or DNA, and may be as long as a full-length MAIAP gene or mRNA.

Any region of the human coding or non-coding MAIAP sequence may be used as a target for antisense inhibition of transcription or translation, and particular sequences for antisense nucleic acids may be selected by well-known approaches. For example, if desired, computer algorithms may be used to identify sequences that form the most stable hybridization duplexes. Computer algorithms may also be used to identify regions of the that are relatively accessible within a folded mRNA molecule; antisense nucleic acids against such regions are more likely to effectively inhibit translation of mRNA. Computer algorithms that may be used to identify optimal sequences for generating antisense nucleic acids include, but are not limited to, OLIGO 5.0 from National Biosciences Inc. (http://www.sxst.it/nbi_olg.htm) and MFOLD (http://mfold2.wustl.edu/˜mfold/rna/form1.cgi). References describing algorithms for predicting secondary structure are described in M. Zuker et al. “Algorithms and Thermodynamics for RNA Secondary Structure Prediction: A Practical Guide.” in: RNA Biochemistry and Biotechnology, J. Barciszewski & B. F. C. Clark, eds., NATO ASI Series, Kluwer Academic Publishers (1999) and in Mathews et al. J. Mol. Biol. 288:911-940 (1999).

Test Compounds

In general, novel drugs for modulation of MAIAP expression or activity (or mimicry of MAIAP activity) may be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are generated, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their cell death-modulatory or radiation sensitivity-modulatory activities should be employed whenever possible.

When a crude extract is found to modulate (i.e., stimulate or inhibit) or mimic MAIAP expression or activity, further fractionation of the positive lead extract is necessary to isolate chemical constituents responsible for the observed effect. Thus, the goal of the extraction, fractionation, and purification process is the careful characterization and identification of a chemical entity within the crude extract having an activity that stimulates or inhibits MAIAP expression or activity (or mimics the same). The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art.

Compounds identified as being of therapeutic value may be subsequently analyzed using animal models for diseases in which it is desirable to increase MAIAP expression or activity, or to mimic MAIAP activity (for example, to increase levels in cells that are susceptible to apoptosis, such as cardiomyocytes in an animal prone to myocardial infarctions), or to decrease or MAIAP expression or activity (for example, in cancer cells in an animal tumor model, thereby rendering the tumor cells more susceptible to apoptosis).

Below are examples of high-throughput systems useful for evaluating the efficacy of a molecule or compound for increasing or decreasing MAIAP expression or activity, or for mimicking its activity.

Assays for Identifying Compounds that Modulate Apoptotic Cell Death

We have shown that MAIAP, a member of the IAP (inhibitor of apoptosis) family, increases cellular resistance to radiation exposure. Since radiation induces apoptosis in normal cells, measurements of MAIAP levels may be used to determine the apoptotic status of cells in a sample. Such measurements may be employed in high-throughput screens for the identification of novel therapeutic compounds that modulate (i.e., stimulate or inhibit) apoptotic death of cells capable of expressing MAIAP.

For example, to identify a novel compound that stimulates apoptosis in tumor cells that overexpress MAIAP, cells that overexpress MAIAP (e.g., tumor cells or cells that are genetically engineered to overexpress MAIAP) may be treated with various test compounds, after which MAIAP mRNA or protein levels may be measured using well-known approaches, such as (but not limited to) RT-PCR (for mRNA) or ELISA (for protein). A decrease in MAIAP expression indicates a pro-apoptotic compound that may then be further tested using appropriate cell culture and animal models for its usefulness as an anti-cancer agent.

Conversely, to identify a novel compound that inhibits apoptosis, cultured cells that are known to undergo apoptosis when exposed to an appropriate pro-apoptotic stimulus are exposed to test compounds either before, after, or concurrent with exposure to the apoptotic stimulus. An increase in MAIAP mRNA or protein levels relative to a control apoptotic sample not treated with the compound indicates an anti-apoptotic compound that may then be further tested for its usefulness in treating diseases or conditions that involve excessive, pathological apoptosis, e.g. (but not limited to), neurodegenerative diseases, retinal degenerative diseases, cardiac degenerative diseases, and transplant rejection.

Various cell culture models of apoptosis are known in the art; any of these may be used to identify anti-apoptotic compounds with potential therapeutic utility. For example, cultured neurons and cardiomyocytes undergo apoptosis when subjected to hypoxic conditions, neurons undergo apoptosis when exposed to high concentrations of glutamine, NMDA, or other neuroexcitatory compounds, and cultured fibroblasts and many other types of cultured cells undergo apoptosis after serum or growth factor withdrawal, staurosporine exposure, DNA damage, or exposure to reactive oxygen species. One of ordinary skill in the art may readily determine which of the known cell culture models would be appropriate for the high-throughput screens of the invention.

In addition, apoptosis may be inhibited by expressing vector-encoded MAIAP within the experimental cells. MAIAP expression may be placed under one of the many known regulatable promoters, such as a promoter that becomes transcriptional active in the presence of a hormone (e.g., a steroid hormone such as estrogen), an antibiotic (such as tetracycline), metal ions (e.g., zinc), heat shock, or hypoxic conditions. An inducible promoter allows the generation of stable cell lines that become more resistant to apoptosis when MAIAP is inducibly expressed. Such cells may be used in high-throughput screens for identification of compounds that decrease MAIAP-mediated resistance to cell death, which may be monitored by any of the many apoptosis detection assays known in the art or disclosed herein.

ELISA for the Detection of Compounds that Modulate Apoptotic Cell Death

Enzyme-linked immunosorbant assays (ELISAs) are easily incorporated into high-throughput screens designed to test large numbers of compounds for their ability to modulate levels of a given protein. When used in the methods of the invention, changes in the level of MAIAP protein in a sample, relative to a control, reflect changes in the apoptotic status of the cells within the sample. Protocols for ELISA may be found, for example, in Ausubel et al., Current Protocols in Molecular Biology, John Wiley & Sons, New York, N.Y., 1998. Lysates from cells treated with test compounds are prepared (see, for example, Ausubel et al., supra), and are loaded onto the wells of microtiter plates coated with “capture” antibodies against MAIAP. Unbound antigen is washed out, and a MAIAP-specific antibody, coupled to an agent to allow for detection, is added. Agents allowing detection include alkaline phosphatase (which can be detected following addition of calorimetric substrates such as p-nitrophenolphosphate), horseradish peroxidase (which can be detected by chemiluminescent substrates such as ECL, commercially available from Amersham) or fluorescent compounds, such as FITC (which can be detected by fluorescence polarization or time-resolved fluorescence). The amount of antibody binding, and hence the level of MAIAP protein within a lysate sample, is easily quantitated on a microtiter plate reader. An increase the level of MAIAP in a treated sample, relative to the level of MAIAP in an untreated sample, indicates a compound that stimulates apoptosis. Conversely, a decrease in the level of MAIAP in a treated sample, relative to the level of MAIAP in an untreated sample, indicates a compound that inhibits apoptosis.

Any person having ordinary skill in the art will understand that the appropriate controls should be included in each assay. The skilled artisan will know which controls to include, depending upon whether a pro-apoptotic compound or an anti-apoptotic compound is being sought, and depending upon the particular cell culture model being used for the assay.

Quantitative PCR of MAIAP mRNA as an Assay for Compounds that Modulate Apoptotic Cell Death

The polymerase chain reaction (PCR), when coupled to a preceding reverse transcription step (RT-PCR), is a commonly used method for detecting vanishingly small quantities of a target mRNA. When performed within the linear range, with an appropriate internal control target (employing, for example, a housekeeping gene such as β-actin or GAPDH), such quantitative PCR provides an extremely precise and sensitive means of detecting slight modulations in mRNA levels. Moreover, this assay is easily performed in a 96-well format, and hence is easily incorporated into a high-throughput screening assay. The appropriate cells (depending upon whether the screen is for pro-apoptotic compounds or anti-apoptotic compounds) are cultured, treated with test compounds, and (if screening for anti-apoptotic compounds) exposed to an appropriate apoptotic stimulus. The cells are then lysed, the mRNA is reverse-transcribed, and the PCR is performed according to commonly used methods (such as those described in Ausubel et al., Current Protocols in Molecular Biology, John Wiley & Sons, New York, N.Y., 1998), using oligonucleotide primers that specifically hybridize with MAIAP mRNA. Changes in the levels of MAIAP RT-PCR product from samples exposed to test compounds, relative to control samples, indicate test compounds with apoptosis-modulating activity, i.e., an increase in the level of MAIAP RT-PCR product indicates a compound that inhibits apoptosis, and, conversely, a decrease in the level of MAIAP RT-PCR product indicates a compound that stimulates apoptosis.

Primer sequences for MAIAP-specific RT-PCR amplification may be selected using any one of the many known primer selection programs, e.g., Primer3 (http://www-genome.wi.mit.edu/cgi-bin/primer/primer3_www.cgi), or by other commonly-known approaches for selecting PCR primers.

Reporter Gene Assays for Compounds that Modulate Apoptotic Cell Death

Assays employing the detection of reporter gene products are extremely sensitive and readily amenable to automation, hence making them ideal for the design of high-throughput screens. Assays for reporter genes may employ, for example, colorimetric, chemiluminescent, or fluorometric detection of reporter gene products. Many varieties of plasmid and viral vectors containing reporter gene cassettes are easily obtained. Such vectors contain cassettes encoding reporter genes such as lacZ/β-galactosidase, green fluorescent protein, and luciferase, among others. A genomic DNA fragment carrying a MAIAP-specific transcriptional control region (e.g., a promoter and/or enhancer) is first cloned using standard approaches (such as those described by Ausubel et al., supra). The DNA carrying the MAIAP transcriptional control region is then inserted, by DNA subcloning, into a reporter vector, thereby placing a vector-encoded reporter gene under the control of the MAIAP transcriptional control region. The activity of the MAIAP transcriptional control region operably linked to the reporter gene can then be directly observed and quantitated as a function of reporter gene activity in a reporter gene assay.

In one embodiment, for example, the MAIAP transcriptional control region could be cloned upstream from a luciferase reporter gene within a reporter vector. This could be introduced into the test cells, along with an internal control reporter vector (e.g., a lacZ gene under the transcriptional regulation of the β-actin promoter). After the cells are exposed to the test compounds and apoptotic stimulus (if testing for anti-apoptotic compounds), reporter gene activity is measured and MAIAP reporter gene activity is normalized to internal control reporter gene activity. An increase in MAIAP reporter gene activity indicates a compound that inhibits apoptosis and a decrease in MAIAP reporter gene activity indicates a compound that stimulates apoptosis.

Interaction Trap Assays

Two-hybrid methods, and modifications thereof, may be used to identify novel proteins that interact with MAIAP, and hence may be, e.g., naturally occurring regulators of MAIAP or downstream targets of MAIAP. Such assays also may be used to screen for compounds that modulate the physical interactions of MAIAP with itself or with other proteins. Regulators of MAIAP, e.g. proteins that interfere with or enhance the interaction between MAIAP and other proteins may identified by the use of a three-hybrid system. Such assays are well-known to skilled artisans, and may be found, for example, in Ausubel et al., supra.

It will be readily apparent to those of ordinary skill in the art that the above-described assays may be modified for the purpose of identifying compounds that modulate the levels and/or biological activity of any of the tumor antigens disclosed herein.

TRAAM

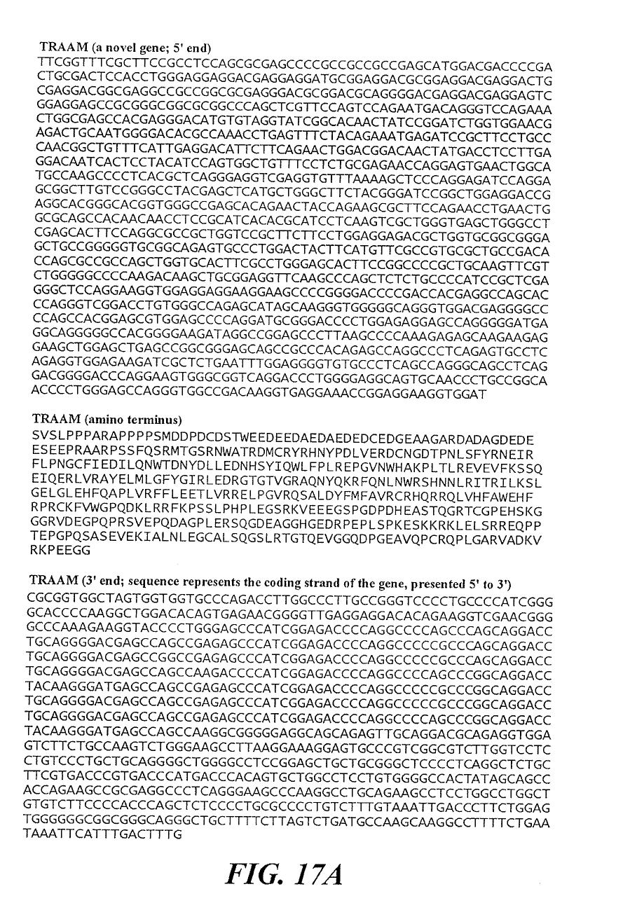

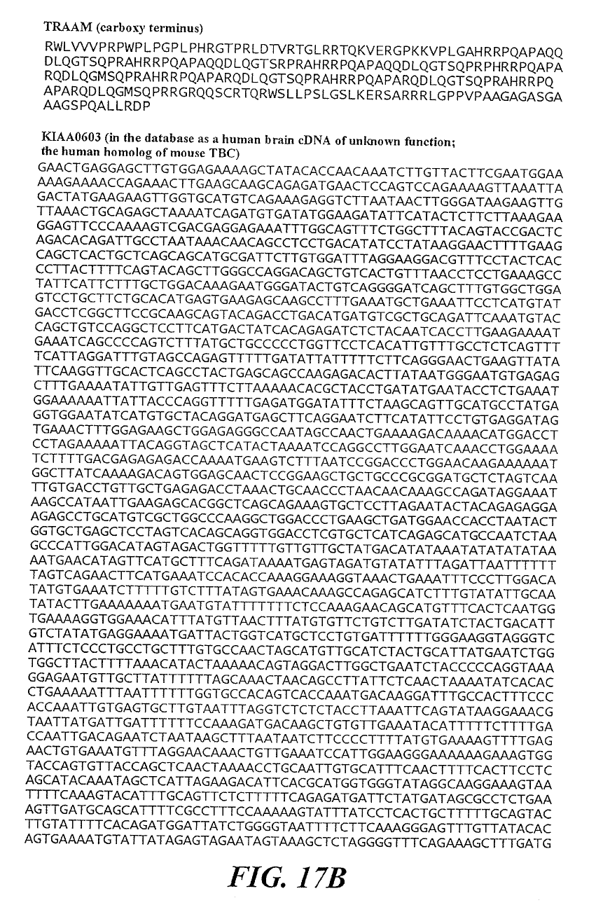

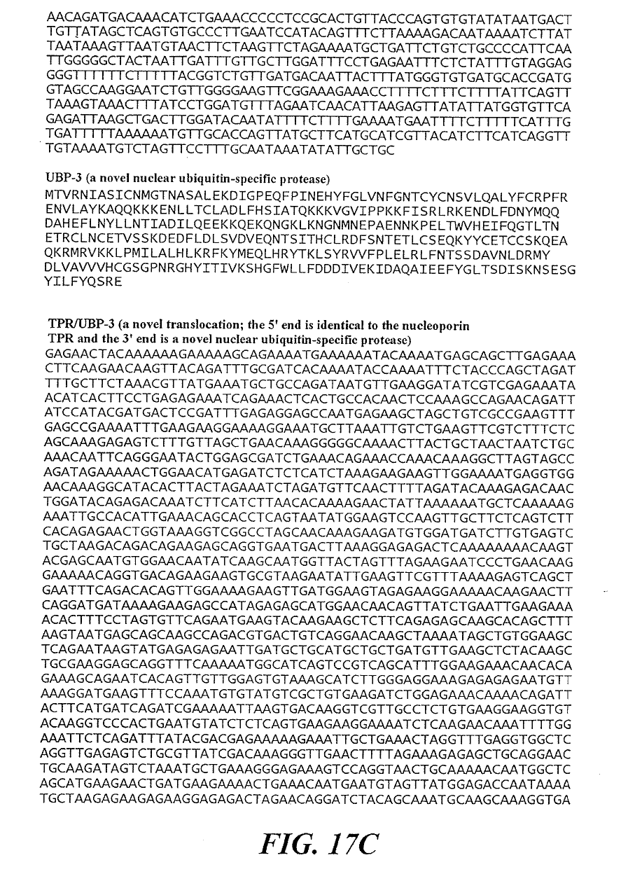

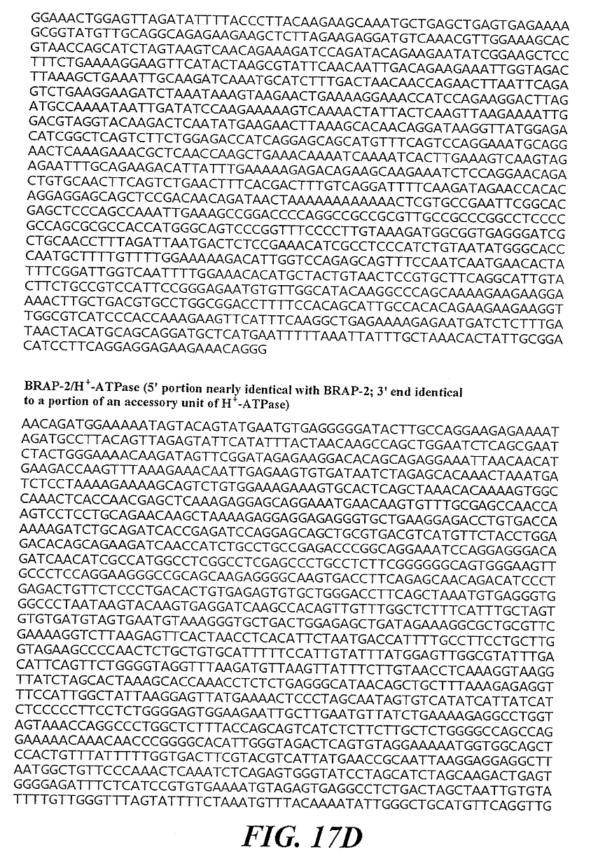

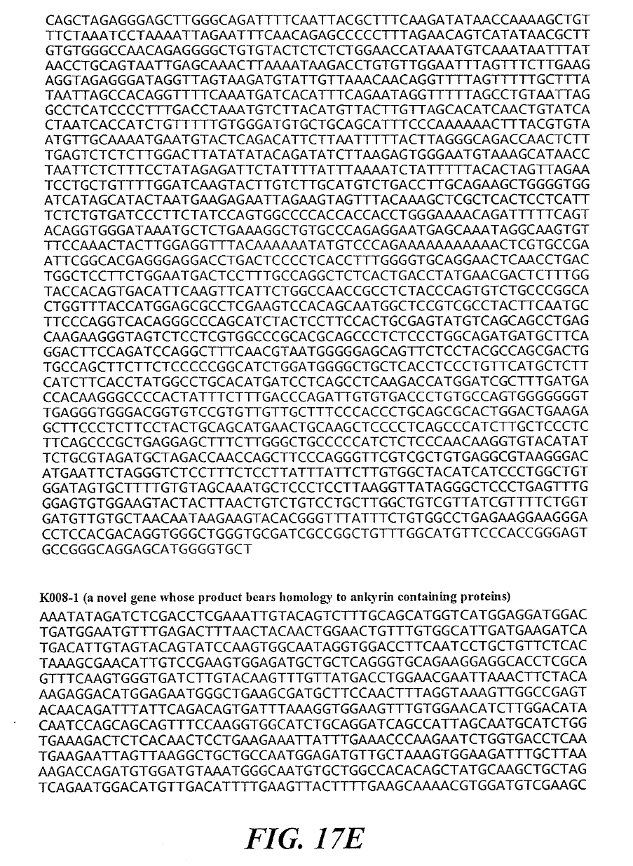

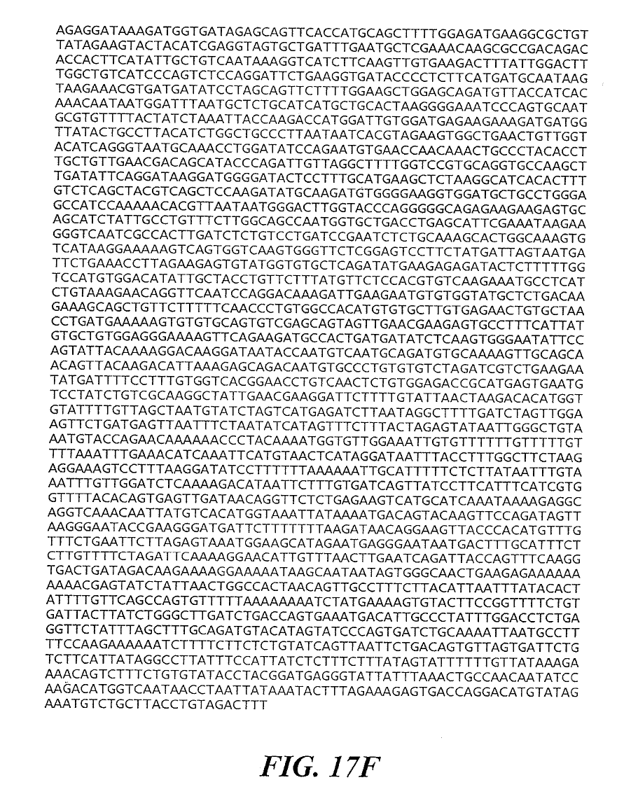

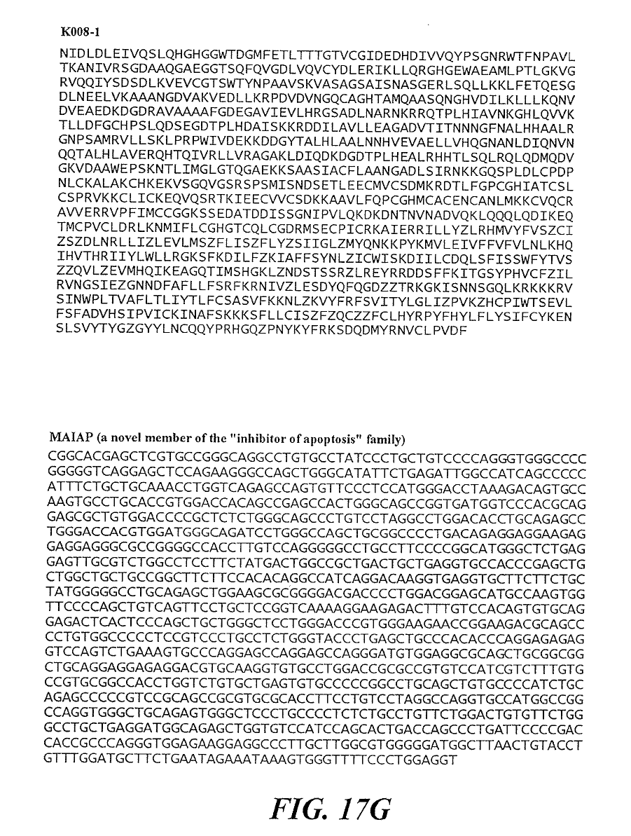

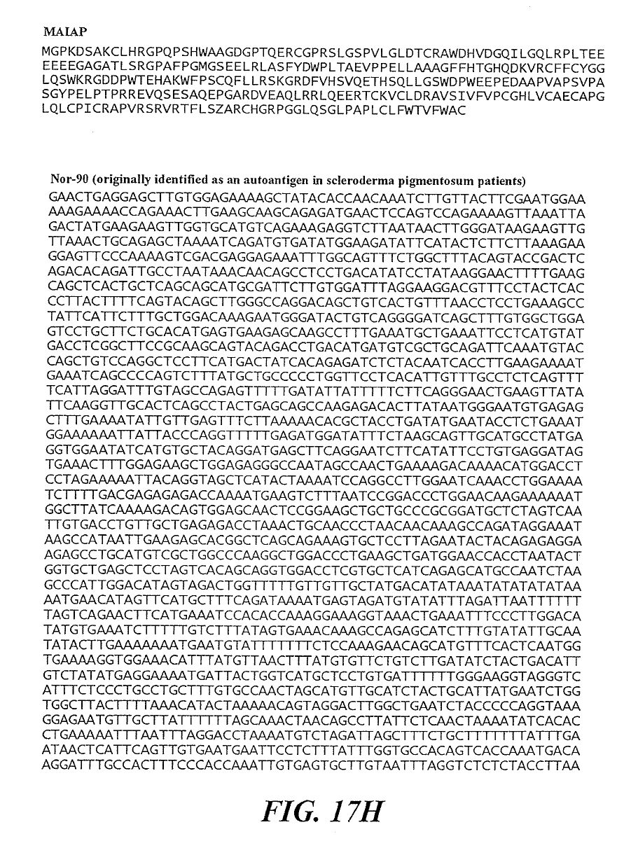

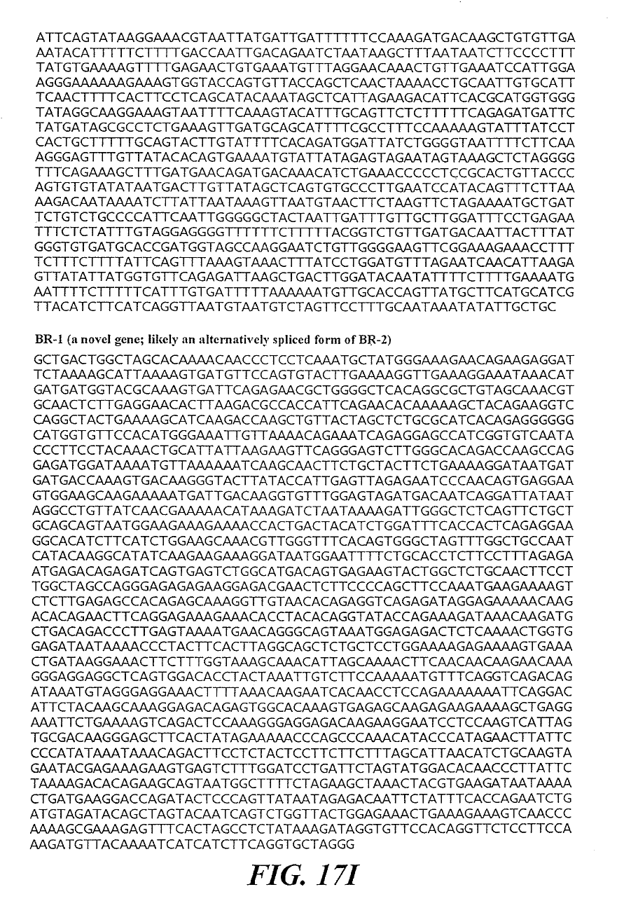

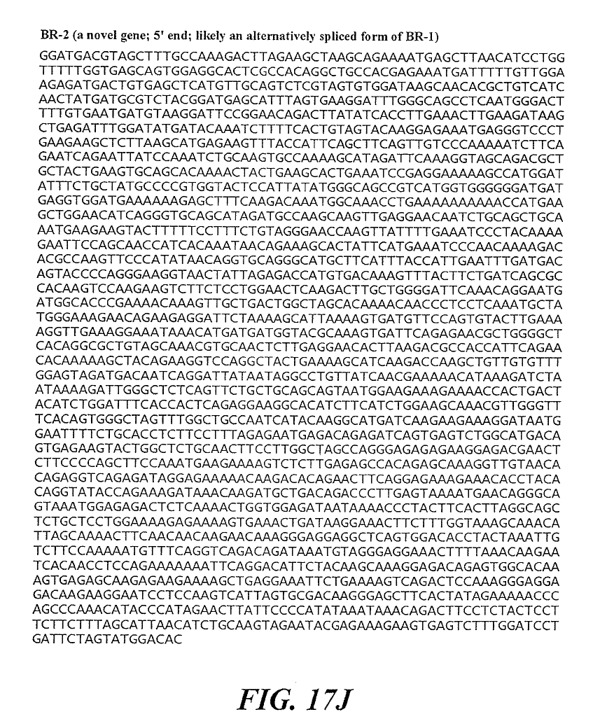

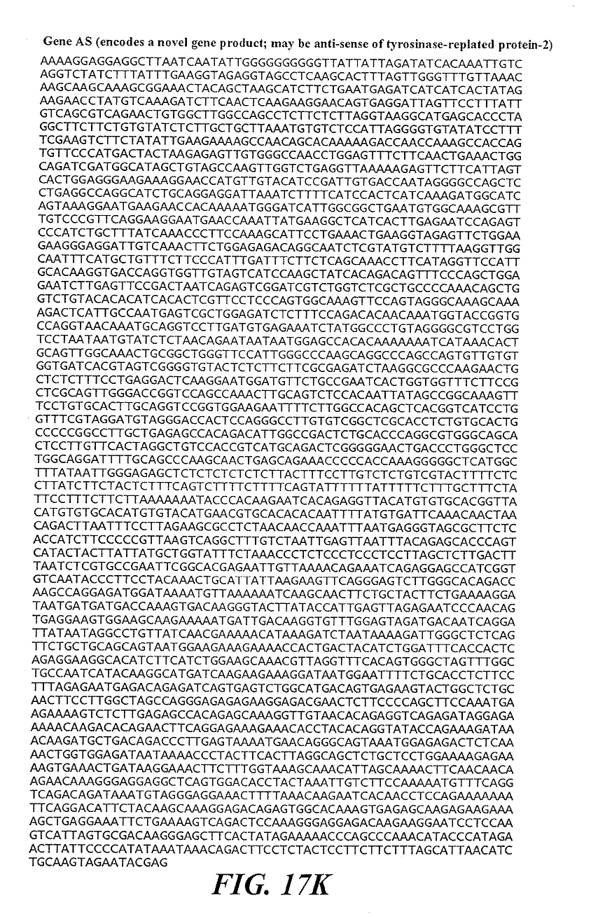

We initially sequenced the 5′ and 3′ ends of a novel gene that we denoted “TRAAM”; see FIG. 17 for the 5′ and 3′ sequences and their encoded polypeptides; SEQ ID NOs: 1-4). The TRAAM clone encodes a novel protein that is structurally related to (yet distinct from) MUC-1, a glycoprotein that is aberrantly glycosylated in tumor cells of epithelial origin and is known to function as a tumor antigen. Our sequence suggested that the putative TRAAM polypeptide contains one partial and six full tandem repeats of twenty amino acids each. The nucleotide sequence matched expressed sequence tags (ESTs) in dbEST (AA865212 (SEQ ID NO: 36); AA641426 (SEQ ID NO: 37); AA399477 (SEQ ID NO: 38); AA486992 (SEQ ID NO: 39); AA076652 (SEQ ID NO: 40); AA293408 (SEQ ID NO: 41); Z25115 (SEQ ID NO: 42); AA079560 (SEQ ID NO: 43); AA534510 (SEQ ID NO: 44)), which were identified in various normal and tumor samples. Our Northern analyses demonstrated expression of this gene in all normal tissues studied. Preliminary analysis suggests that vaccination with GM-CSF-secreting tumor cells stimulates reactivity to this antigen: post-vaccination serum from two melanoma patients contained antibodies that specifically bound TRAAM.

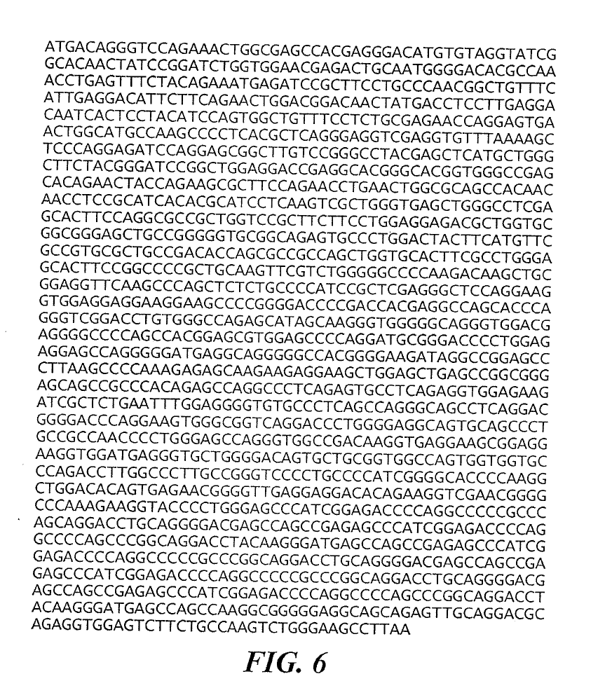

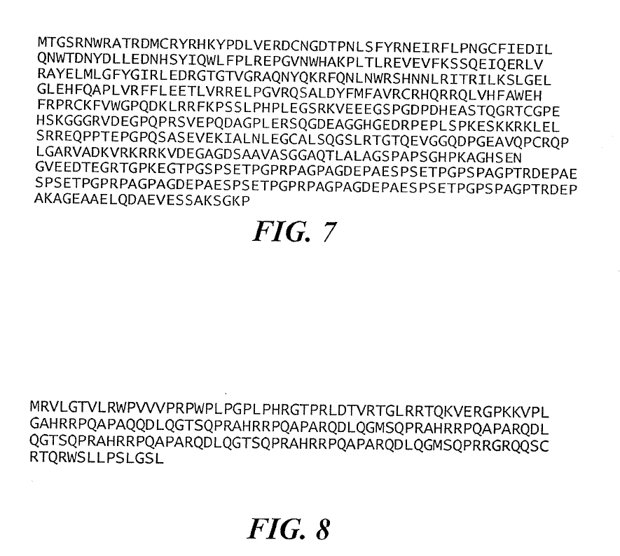

Our initial TRAAM clone (TRAAM_{K008}) was isolated from a phage expression library prepared from K008 melanoma cells; this cell line was derived from a patient sample. To complete the TRAAM nucleotide sequence, we used a probe derived from TRAAM_{K008} to obtain a TRAAM clone (TRAAM_{U987}) from a cDNA library prepared from the U937 macrophage cell line. While we were completing the TRAAM_{U987} sequence, it became apparent that the open reading frame (ORF) suggested by our TRAAM_{K008} 5′ and 3′ sequences did not fully line up with the longest ORF that we inferred from the full-length TRAAM_{U987} sequence. This suggested the possibility that two distinct proteins might be encoded by the TRAAM gene. In fact, our translation (SEQ ID NO: 2; FIG. 17) of the TRAAM_{K008} 5′ sequence (SEQ ID NO: 1; FIG. 17) is similar to the translation of the longer TRAAM_{U987} ORF (TRAAM_{U987}-ORF1; SEQ ID NO: 18; FIG. 7) and our translation (SEQ ID NO: 4; FIG. 17) of the TRAAM_{K008} 3′ sequence (SEQ ID NO: 3; FIG. 17) is similar to the translation of the shorter TRAAM_{U987} ORF (TRAAM_{U987}-ORF2; SEQ ID NO: 19; FIG. 8). The translation of TRAAM_{U987}-ORF1 begins on nucleotide 1 of SEQ ID NO: 17 (FIG. 6), and the translation of TRAAM_{U987}-ORF2 begins on nucleotide 1208 of SEQ ID NO: 17 (FIG. 6).

To test the hypothesis that TRAAM_{U987} encodes two polyepeptides, we generated two peptides that correspond to tandem repeat regions within TRAAM_{U987}-ORF 1 and TRAAM_{U987}-ORF2. The first peptide, which was derived from the TRAAM_{U987}-ORF1 amino acid sequence (SEQ ID NO: 18; FIG. 7) has the amino acid sequence SPSETPGPRPAGPAGDEPAESPSETPGPRPAGPAGDEPAKTPSETPGPS (SEQ ID NO: 20); we call this peptide “PSET”. The second peptide, which was derived from the TRAAM_{U987}-ORF2 amino acid sequence (SEQ ID NO: 19; FIG. 8) has the amino acid sequence AHRRPQAPAQQDLQGTSQPRAHRRPQAPAQQDLQGTSQPRAHRRPQAP AQ (SEQ ID NO: 21); we call this peptide “TRAM.”

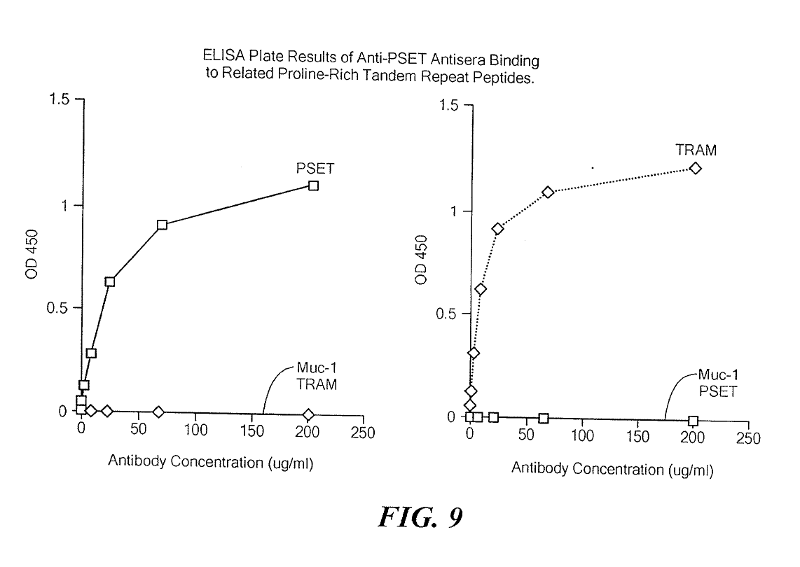

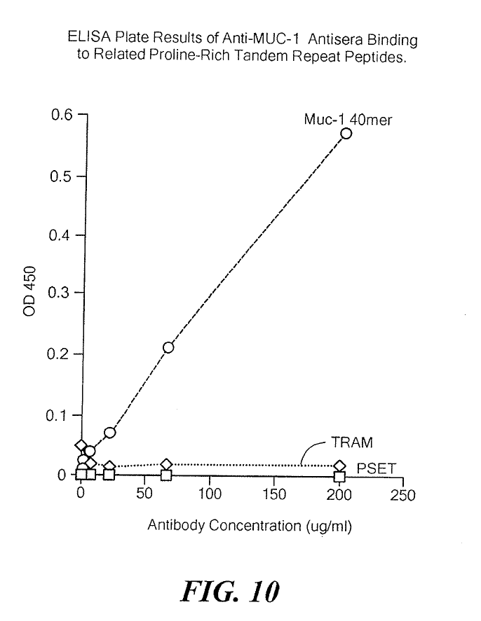

Rabbits were immunized with the two peptides and high-titer polyclonal antisera were obtained. The peptides and antibodies were used to set up ELISA assays. FIG. 9 shows the results of ELISA assays with anti-PSET or anti-TRAM antibodies and peptides encompassing the tandem repeats of PSET, TRAM, and MUC-1. The left panel of FIG. 9 shows that the antibodies generated against the PSET-derived peptide do not cross-react with the TRAM-derived peptide or the MUC-1-derived peptide. Likewise, the right panel of FIG. 9 shows that the antibodies generated against the TRAM-derived peptide do not cross-react with the PSET-derived peptide or the MUC-1-derived peptide. FIG. 10 is a diagram of a control ELISA showing that anti-MUC-1 antiserum does not detect PSET or TRAM.

The full-length TRAAM_{U987} cDNA was cloned into a vector such that the encoded PSET and TRAM proteins each carry a histidine tag; the cDNA was then expressed in bacteria. Bacterial lysates contained a protein that was recognized only by the PSET-specific sera and a second protein that was recognized only by the TRAM-specific sera. This shows that two distinct protein products, PSET and TRAM, are translated from TRAAM_{U987} in bacteria.

Western analyses using lysates prepared from K008 melanoma cells and U937 macrophages showed that anti-TRAAM antisera (from rabbits immunized with the TRAM peptide) recognized proteins that were distinct from proteins recognized by anti-PSET antisera (from rabbits immunized with the PSET peptide). Our results indicate that the TRAAM gene encodes at least two polypeptides, PSET and TRAM. We are in the process of generating a retroviral vector encoding full-length TRAAM_{U987} to provide additional confirmation that the two translation products are expressed in eukaryotic cells. It is possible that there are differences between cancer and normal cells in this dual expression; such differences may prove to be valuable for diagnostic assays and therapeutic strategies. We are in the process of determining, by ELISA, whether either of these two proteins are recognized by patient sera.

In comparing our translation of our TRAAM_{K008} 3′ sequence with TRAAM_{U987}-ORF-2 (which contains the TRAM peptide), we observed that while TRAAM_{K008} contained one partial and six full repeats of the twenty amino acid repeat sequence, TRAAM_{U987} contained one partial and only four repeats of the repeat sequence. Since MUC-1 (which has a similar repeat sequence) from different individuals has been shown to contain a highly variable number (ranging from single digit numbers to triple digit numbers) of repeat sequences, it is likely that PSET-containing and TRAM-containing polypeptides encoded by the TRAAM gene (e.g., TRAAM-ORF1 and TRAAM-ORF2, respectively) will also contain a variable number of repeats. The consensus sequence for the twenty amino acid TRAM (TRAAM_{U987}-ORF1 and TRAAM_{K008} carboxy-terminus)repeat sequence is: G T/m S Q/r P R A/p H R R P Q A P A R Q Q D L Q (SEQ ID NO: 24). The sequence of the partial TRAM repeats is: A H R R P Q A P A Q Q D L Q (SEQ ID NO: 25). Other preferred TRAM repeat sequences are:

(SEQ ID NO:26)

G T S Q P R A H R R P Q A P A R Q D L Q;

(SEQ ID NO:27)

G M S Q P R A H R R P Q A P A R Q D L Q;

(SEQ ID NO:28)

G T S Q P R A H R R P Q A P A Q Q D L Q;
and

(SEQ ID NO:29)

G T S Q P R P H R R P Q A P A R Q D L Q.

The consensus sequence for the PSET twenty amino acid repeat sequence is:

(SEQ ID NO:29)

S P S E T P G P R/S P A G P A/T G/R D E P A E/k.

Preferred sequences are:

(SEQ ID NO:31)

S P S E T P G P R P A G P A G D E P A E;

(SEQ ID NO:32)

S P S E T P G P S P A G P T R D E P A E;
and

(SEQ ID NO:33)

S P S E T P G P S P A G P T R D E P A K.

Peptides corresponding to the TRAAM (e.g., PSET and TRAM) repeat sequences will be particularly useful as tumor antigens and as therapeutic peptides that block the activity of TRAAM gene products. Antisense nucleic acids that are complementary to the nucleotide sequences encoding the repeats will be useful for antisense inhibition of transcription and/or translation of the TRAAM gene.

3. KIAA0603/TBC

We isolated a cDNA clone, the sequence of which suggested that it encoded the human homolog of the mouse TBC-1 protein. While we were in the process of obtaining the full-length sequence of this clone, the gene KIAA0603 (Genbank Accession No. AB011175; Nagase et al., DNA Res. 5:31-39, 1998; SEQ ID NOs: 45 and 46), which corresponds to our sequence, was deposited in the database. Our sequence also corresponds to ESTs reported from normal and neoplastic tissues. We observed that serum from a second vaccinated patient also identified this protein as a tumor antigen.

4. TPR/UBP-3

TPR/UBP-3 (SEQ ID NO: 7) is a novel translocation in which the 5′ partner encodes the TPR protein (a nucleoporin); the 3′ partner encodes a novel gene (designated UBP-3; SEQ ID NO: 6) that is likely the human homolog of a ubiquitin-specific protease found in Arabidopsis thaliana. Post-vaccination serum from eleven melanoma patients contained antibodies that specifically bound the TPR/UBP-3 fusion product.

5. BRAP-2/H⁺-ATPase

BRAP-2/H⁺-ATPase (SEQ ID NO: 8) is a novel translocation in which the 5′ partner is similar to BRAP-2, a protein reported to be a binding partner of DDB p 127 (the Xeroderma pigmentosum group E defective protein), as well as a binding partner of BRCA-1 (GenBank AF035620; J. Biol. Chem. 273:6183-6189, 1998; SEQ ID NOs: 47 and 48); there is a one-base-pair discrepancy between our sequence and the reported BRAP-2 sequence, which results in a different BRAP-2 carboxy terminus from that reported. The 3′ partner is an accessory protein reported to be associated with the H+ vacuolar ATPase. Northern analysis shows expression of this ATPase subunit in all normal tissues examined. Nine post-vaccination serum samples contained antibodies that recognized the BRAP-2/H⁺-ATPase fusion protein. Preliminary Western analysis suggests that the melanoma patient whose sera was used to clone this gene recognizes the ATPase subunit at high levels, but not BRAP-2. Recombinant BRAP-2 and the ATPase subunit have each been produced in bacteria as GST fusion proteins.

6. KOO8-1

KOO8-1 (SEQ ID NOs: 9 and 10) is a novel gene sequence with several matches in the EST data base (AA459-487 (SEQ ID NO: 50); AA612844 (SEQ ID NO: 51); AA742366 (SEQ ID NO: 52); AA280738 (SEQ ID NO: 53); AA578562 (SEQ ID NO: 54); AA665027 (SEQ ID NO: 55); D81704 (SEQ ID NO: 56); AA748357 (SEQ ID NO: 57); D60753 (SEQ ID NO: 58); AA972891 (SEQ ID NO: 59)). There is a weak homology with ankyrin repeats at the protein level.

7. NOR-90

NOR-90 is a gene encoding a protein that has previously been reported as an autoantigen in patients with scleroderma, a connective tissue disease (J. Exp. Med. 174:1239-1244, 1991; Genebank Accession No. X56687; SEQ ID NO: 60). This protein is known to function as a transcription factor for rRNA synthesis. Our clone corresponds to a portion of NOR-90. Five out of seven post-vaccination serum samples contained antibodies that recognize NOR-90.

8. BR-1

BR-1 (SEQ ID NO: 14) is a novel gene sequence for which there are two EST matches: GB AA187982 (from HeLa cells; SEQ ID NO: 62) and GB AA354716 (from Jurkat T cells; SEQ ID NO: 63).

9. BR-2

BR-2 (SEQ ID NO: 15) is a novel gene sequence for which there are three EST matches: GB AA187982 (SEQ ID NO: 64), AA88110 (SEQ ID NO: 65), and EMB Z21827 (SEQ ID NO: 66). BR-1 and BR-2 may be alternative splice products of the same gene.

10. Gene AS

Gene AS (SEQ ID NO: 16) is a novel gene sequence that appears to be in the antisense orientation for the gene encoding tyrosinase-replated protein-2, a melanoma antigen.

In addition, TPR (Accession Number: EMB X66397; SEQ ID NO: 67), UBP-3 (SEQ ID NO: 6), and BRAP-2 (encoded either by our sequence, or by the previously-reported sequence) are considered to be tumor antigens for use in the methods of the invention. Novel gene sequences are shown in FIGS. 6 through 8 and 17.

Uses of the Invention

1. Diagnostic: Either DNA- or antibody-based tests may be used for detection of the translocations, alterations in the level of expression, or modifications (such as mutations) of the tumor antigen genes or gene products. Monitoring antibody or cellular responses to these antigens may be useful in determining disease burden, prognosis, and response to cancer therapy. For example, a patient's response to an anti-tumor vaccine may be monitored by measuring the levels of tumor antigen-specific antibodies or tumor antigen-specific cytotoxic T lymphocytes in a sample obtained from the patient before and after vaccination, using an appropriate assay such as ELISA (for antibody detection) or cytotoxic T lymphocyte functional assays, such as those measuring cytotoxicity, proliferation, or cytokine production.

2. Immunotherapies: The tumor antigens of the invention were identified as the targets of high titer IgG-specific antibody responses. There is a high likelihood that they will also be the target for helper and/or cytotoxic T cells, given the concurrent induction of humoral and cellular immunity in our vaccinated patients.
a. The tumor antigens may be used as components of generic vaccines in immunization strategies that employ, e.g.: peptides, whole proteins (alone, or with a wide variety of adjuvants including, but not limited to, QS21, alum, GMCSF, IL-2, IL-12), naked DNA, viral vectors (i.e., adenovirus, vaccinia virus, fowlpox) expressing antigen, dendritic cells (pulsed with peptide or protein or genetically modified to express the relevant tumor antigen), cell lines engineered to express these antigens with or without adjuvants (such as QS21, GM-CSF, IL-2, IL-12) as well as cell lines engineered to express immunostimulatory molecules (such as GM-SCF, B7-1, IL-2, IL-12).
b. Monoclonal antibodies against tumor antigens may be used not only for diagnosis (as in 1 above) but also therapeutically (for example, conjugated to a variety of toxins).
c. Antigen-specific T cells generated against these antigens are likely to be useful for adoptive immunotherapy.

3. Pharmacologic therapies: These antigens may represent targets for drug therapy. The tumor antigens encode proteins that are likely to be important to the cancer cell phenotype. For example, TBC-1 is a nuclear protein with structural homology to cell cycle regulators. As a second example, MAIAP, a member of the “inhibitor of apoptosis” family of proteins, may be overexpressed in some tumor cells. If so, inhibition of MAIAP, e.g., using antisense nucleic acids or small molecule inhibitors, may increase the effectiveness of apoptosis-inducing cancer therapies, such as chemotherapy or radiation therapy, for some types of tumors.

4. The tumor antigens may also be used as targets for high-throughput drug screens designed to isolate small molecule inhibitors of tumor antigen function, using known methods and as described herein. For example, high-throughput screens designed to detect inhibitors of MAIAP allow the identification of novel cancer therapeutics that may be used alone or in conjunction with other cancer therapies.

Synthesis of Tumor Antigen Polypeptides

Cloned tumor antigens may be overexpressed in vivo by introducing tumor antigen coding sequences into various types of cells, or in vitro, using cell-free expression systems that are known in the art. Tumor antigen gene products may then purified for biochemical characterization, antibody or vaccine production, or patient therapy. Purified tumor antigens are also useful for diagnostic assays that measure the presence of antibodies, e.g., in a patient's serum, that are specific for a given tumor antigen. The presence (or increased levels) of anti-tumor antigen antibodies in a patient's serum, relative to a reference sample, may indicate that the patient has a tumor or a tumor metastasis.

Eukaryotic and prokaryotic expression systems may be used to express tumor antigen proteins: tumor antigen gene sequences are introduced into a plasmid or other vector, which is then used to transform living cells. Constructs in which a tumor antigen cDNA containing the entire open reading frame, inserted in the correct orientation into an expression plasmid, may be used for protein expression. Alternatively, portions of tumor antigen gene sequences, including wild-type or mutant tumor antigen sequences, may be inserted. Prokaryotic and eukaryotic expression systems allow various immunogenic domains of tumor antigen proteins to be recovered as fusion proteins and then used for the generation of appropriate antibodies. In some cases, for example, when a tumor antigen is to be expressed directly within a patient's cells, it may be desirable to express the tumor antigen under the control of an inducible or tissue-specific promoter.

Typical expression vectors contain promoters that direct the synthesis of large amounts of mRNA corresponding to the inserted tumor antigen-encoding nucleic acid in the plasmid-bearing cells. They may also include eukaryotic or prokaryotic “origin of replication” sequences allowing for their autonomous replication within the host organism, sequences that encode genetic traits that allow vector-containing cells to be selected for in the presence of otherwise-toxic drugs (such as antibiotics), and sequences that increase the efficiency with which the synthesized mRNA is translated. Stable long-term vectors may be maintained as freely replicating entities within cells by using regulatory elements of, for example, viruses (e.g., the OriP sequences from the Epstein Barr Virus genome). Cell lines may also be produced that have integrated the vector into the genomic DNA, and in this manner the gene product is produced on a continuous basis.

Expression of foreign sequences in bacteria such as Escherichia coli requires the insertion of a nucleic acid sequence encoding a polypeptide into a bacterial expression vector. Plasmid vectors in this category contain several elements required for the propagation of the plasmid in bacteria, and expression of inserted DNA of the plasmid by the plasmid-carrying bacteria. Propagation of only plasmid-bearing bacteria is achieved by introducing, into the plasmid, selectable marker-encoding sequences that allow plasmid-bearing bacteria to grow in the presence of otherwise-toxic drugs (e.g., antibiotics). The plasmid also bears a transcriptional promoter capable of producing large amounts of mRNA from the cloned gene. Such promoters may or may not be inducible promoters that initiate transcription upon induction. The plasmid also preferably contains a polylinker to simplify insertion of the gene in the correct orientation within the vector. In a simple E. coli expression vector utilizing the lac promoter, the expression vector plasmid contains a fragment of the E. coli chromosome containing the lac promoter and the neighboring lacZ gene. In the presence of the lactose analog IPTG, RNA polymerase normally transcribes the lacZ gene, producing lacZ mRNA, which is translated into the encoded protein, β-galactosidase. The lacZ gene can be cut out of the expression vector with restriction endonucleases and replaced by a tumor antigen gene sequence, or fragment, fusion, or mutant thereof. When this resulting plasmid is transfected into E. coli, addition of IPTG and subsequent transcription from the lac promoter produces mRNA encoding the polypeptide of interest, e.g., a tumor antigen, which is translated into a polypeptide, e.g., tumor antigen polypeptide.

Once the appropriate expression vectors containing a tumor antigen gene (or fragment, fusion, or mutant thereof) are constructed, they are introduced into an appropriate host cell by transformation, transfection, or transduction techniques that are known in the art, including calcium chloride transformation, calcium phosphate transfection, DEAE-dextran transfection, electroporation, microinjection, protoplast fusion and liposome-mediated transfection. The host cells that are transformed with the vectors of this invention may include (but are not limited to) E. coli or other bacteria, yeast, fungi, insect cells (using, for example, baculoviral vectors for expression), human, mouse, or other animal cells. Mammalian cells can also be used to express tumor antigen proteins using a vaccinia virus expression system described in F. Ausubel et al., Current Protocols in Molecular Biology, John Wiley & Sons, New York, N.Y., 1994.

In vitro expression of tumor antigen proteins, fusions, polypeptide fragments, or mutated versions thereof encoded by cloned DNA is also possible using the T7 late-promoter expression system. This system depends on the regulated expression of T7 RNA polymerase, an enzyme encoded in the DNA of bacteriophage T7. The T7 RNA polymerase transcribes DNA beginning within a specific 23-bp promoter sequence called the T7 late promoter. Copies of the T7 late promoter are located at several sites on the T7 genome, but none is present in E. coli chromosomal DNA. As a result, in T7-infected cells, T7 RNA polymerase catalyzes transcription of viral genes but not of E. coli genes. In this expression system, recombinant E. coli cells are first engineered to carry the gene encoding T7 RNA polymerase under the transcriptional regulation of the lac promoter. In the presence of IPTG, these cells transcribe the T7 polymerase gene at a high rate and synthesize abundant amounts of T7 RNA polymerase. These cells are then transformed with plasmid vectors that carry a copy of the T7 late promoter protein. When IPTG is added to the culture medium containing these transformed E. coli cells, large amounts of T7 RNA polymerase are produced. The polymerase then binds to the T7 late promoter on the plasmid expression vectors, catalyzing transcription of the inserted cDNA at a high rate. Since each E. coli cell contains many copies of the expression vector, large amounts of mRNA corresponding to the cloned cDNA can be produced in this system and the resulting protein can be radioactively labeled.

Plasmid vectors containing late promoters and the corresponding RNA polymerases from related bacteriophages such as T3, T5, and SP6 may also be used for in vitro production of proteins from cloned DNA. E. coli can also be used for expression using an M13 phage such as mGPI-2. Furthermore, vectors that contain phage lambda regulatory sequences, or vectors that direct the expression of fusion proteins, for example, a maltose-binding protein fusion protein or a glutathione-S-transferase fusion protein, also may be used for expression in E. coli.

Eukaryotic expression systems permit appropriate post-translational modifications to expressed proteins. Transient transfection of a eukaryotic expression plasmid allows the transient production of a tumor antigen polypeptide by a transfected host cell. Tumor antigen proteins may also be produced by a stably-transfected mammalian cell line. A number of vectors suitable for stable transfection of mammalian cells are available to the public (e.g., see Pouwels et al., Cloning Vectors: A LaboratoryManual, 1985, Supp. 1987), as are methods for constructing such cell lines (see e.g., F. Ausubel et al., Current Protocols in Molecular Biology, John Wiley & Sons, New York, N.Y., 1994). In one example, cDNA encoding a tumor antigen protein, fusion, mutant, or polypeptide fragment is cloned into an expression vector that includes the dihydrofolate reductase (DHFR) gene. Integration of the plasmid and, therefore, integration of the tumor antigen-encoding gene into the host cell chromosome is selected for by inclusion of 0.01-300 μM methotrexate in the cell culture medium (described in F. Ausubel et al., supra). This dominant selection can be accomplished in most cell types. Recombinant protein expression can be increased by DHFR-mediated amplification of the transfected gene. Methods for selecting cell lines bearing gene amplifications are described in F. Ausubel et al., supra. These methods generally involve extended culture in medium containing gradually increasing levels of methotrexate. The most commonly used DHFR-containing expression vectors are pCVSEII-DHFR and pAdD26SV(A) (described in F. Ausubel et al., supra). The host cells described above or, preferably, a DHFR-deficient CHO cell line (e.g., CHO DHFR⁻ cells, ATCC Accession No. CRL 9096) are among those most preferred for DHFR selection of a stably-transfected cell line or DHFR-mediated gene amplification. Other drug markers may be analogously used.

Eukaryotic cell expression of proteins, such as tumor antigens, allows the production of large amounts of normal or mutant proteins for isolation and purification, and the use of cells expressing a tumor antigen protein provides a functional assay system for antibodies generated against the protein of interest. Expression of tumor antigen proteins, fusions, mutants, and polypeptide fragments in eukaryotic cells also enables studies of the functions of the normal complete proteins, specific portions of the proteins, or of naturally occurring polymorphisms and artificially produced mutated proteins. The tumor antigen-encoding DNA sequences can be altered using procedures known in the art, such as restriction endonuclease digestion, DNA polymerase fill-in, exonuclease deletion, terminal deoxynucleotide transferase extension, ligation of synthetic or cloned DNA sequences and site-directed sequence alteration using specific oligonucleotides together with PCR.

Another preferred eukaryotic expression system is the baculovirus system using, for example, the vector pBacPAK9, which is available from Clontech (Palo Alto, Calif.). If desired, this system may be used in conjunction with other protein expression techniques, for example, the myc tag approach described by Evan et al. (Mol. Cell. Biol. 5:3610-3616, 1985).

Once the recombinant protein is expressed, it can be isolated from the expressing cells by cell lysis followed by protein purification techniques, such as affinity chromatography. In this example, an anti-tumor antigen antibody, which may be produced by the methods described herein, can be attached to a column and used to isolate recombinant tumor antigen proteins. Lysis and fractionation of tumor antigen protein-harboring cells prior to affinity chromatography may be performed by standard methods (see e.g., F. Ausubel et al., Current Protocols in Molecular Biology, John Wiley & Sons, New York, N.Y., 1994). Once isolated, the recombinant protein can, if desired, be purified further, e.g., by high performance liquid chromatography (HPLC; e.g., see Fisher, Laboratory Techniques In Biochemistry And Molecular Biology, Work and Burdon, Eds., Elsevier, 1980).

Polypeptides of the invention, particularly tumor antigen polypeptide fragments, can also be produced by chemical synthesis (e.g., by the methods described in Solid Phase Peptide Synthesis, 2nd ed., 1984, The Pierce Chemical Co., Rockford, Ill.). These general techniques of polypeptide expression and purification can also be used to produce and isolate useful tumor antigen polypeptide fragments or analogs, as described herein.

Those skilled in the art of molecular biology will understand that a wide variety of expression systems may be used to produce the recombinant tumor antigen polypeptides and fragments thereof. Tumor antigen polypeptides may be produced in prokaryotic hosts (e.g., E. coli) or in eukaryotic hosts (e.g., S. cerevisiae, insect cells such as Sf9 cells, or mammalian cells such as COS-1, NIH 3T3, or HeLa cells). These cells are commercially available from, for example, the American Type Culture Collection, Rockville, Md. (see also F. Ausubel et al., supra). The method of transformation and the choice of expression vehicle (e.g., expression vector) will depend on the host system selected. Transformation and transfection methods are described, e.g., in F. Ausubel et al., supra, and expression vehicles may be chosen from those provided, e.g., in Pouwels et al., Cloning Vectors: A Laboratory Manual, 1985, Supp. 1987.

Anti-Tumor Antigen Antibodies

In order to prepare purified polyclonal antibodies, tumor antigens, fragments of tumor antigens, or fusion proteins containing defined portions of tumor antigens can be synthesized in bacteria by expression of corresponding DNA sequences cloned into a suitable vector. Fusion proteins are commonly used as a source of antigen for producing antibodies. Two widely used expression systems for E. coli are lacZ fusions using the pUR series of vectors and trpE fusions using the pATH vectors. The proteins can be purified, and then coupled to a carrier protein and mixed with Freund's adjuvant (to stimulate the antigenic response by the animal of choice) and injected into rabbits or other laboratory animals. Alternatively, protein can be isolated from tumor antigen-expressing cultured cells. Following booster injections at bi-weekly intervals, the rabbits or other laboratory animals are then bled and the sera isolated. The sera can be used directly or can be purified prior to use, by various methods, including affinity chromatography employing reagents such as Protein A-Sepharose, Antigen Sepharose, and Anti-mouse-Ig-Sepharose. The sera can then be used to probe protein extracts from tumor antigen-expressing tissue, for example, by immunoprecipitation of tumor antigen from whole extracts, or by Western blotting of extracts that have been electrophoretically separated. Alternatively, synthetic peptides can be made that correspond to the antigenic portions of the protein, and used to innoculate the animals.

In order to generate peptide or full-length protein for use in making tumor antigen-specific antibodies, a tumor antigen coding sequence can be expressed as a C-terminal fusion with glutathione S-transferase (GST; Smith et al., Gene 67:31-40, 1988). The fusion protein may be purified on glutathione-Sepharose beads, eluted with glutathione, and cleaved with thrombin (at an engineered cleavage site), and purified to the degree required to successfully immunize rabbits. Primary immunizations may be carried out with Freund's complete adjuvant and subsequent immunizations performed with Freund's incomplete adjuvant. Antibody titers may be monitored by Western blot and immunoprecipitation analyses using the thrombin-cleaved tumor antigen fragment of the GST-tumor antigen fusion protein. Immune sera may be affinity purified using CNBr-Sepharose-coupled tumor antigen protein. Antiserum specificity may be determined using a panel of unrelated GST fusion proteins.

It is also understood by those skilled in the art that monoclonal tumor antigen-specific antibodies may be produced by using tumor antigen isolated from tumor antigen-expressing cultured cells, or tumor antigen isolated from tissues (such as tumors). The cell extracts, or recombinant protein extracts containing tumor antigen, may for example, be injected with Freund's adjuvant into mice. After injection, spleens are removed from the mice and isolated spleen cells are suspended, e.g., in phosphate buffered saline (PBS). The spleen cells serve as a source of lymphocytes, some of which produce antibody of the appropriate specificity. These are then fused with permanently growing myeloma partner cells, and the products of the fusion are plated into tissue culture wells in the presence of a selective agent such as hypoxanthine, aminopterine, and thymidine (HAT). The wells are then screened by ELISA to identify those containing cells making antibody capable of binding a tumor antigen or polypeptide fragment or mutant thereof. These are then re-plated and after a period of growth, these wells are again screened to identify antibody-producing cells. Several cloning procedures are carried out until over 90% of the wells contain single clones which are positive for antibody production. From this procedure a stable line of clones that produce the antibody is established. The monoclonal antibody can then be purified by affinity chromatography using Protein A Sepharose, ion-exchange chromatography, as well as variations and combinations of these techniques. Truncated versions of monoclonal antibodies may also be produced by recombinant methods in which plasmids are generated which express the desired monoclonal antibody fragment(s) in a suitable host.

As an alternate or adjunct immunogen to GST fusion proteins, peptides corresponding to relatively unique hydrophilic regions of a tumor antigen may be generated and coupled to keyhole limpet hemocyanin (KLH) through an introduced C-terminal lysine. Antiserum to each of these peptides is similarly affinity purified on peptides conjugated to BSA, and specificity is tested by ELISA and Western blotting using peptide conjugates, and by Western blotting and immunoprecipitation using the tumor antigen of interest expressed as a GST fusion protein.

Alternatively, monoclonal antibodies that specifically bind the tumor antigen proteins described herein may be prepared using standard hybridoma technology (see, e.g., Kohler et al., Nature 256:495, 1975; Kohler et al., Eur. J. Immunol. 6:511, 1976; Kohler et al., Eur. J. Immunol. 6:292, 1976; Hammerling et al., In Monoclonal Antibodies and T Cell Hybridomas, Elsevier, New York, N.Y., 1981; F. Ausubel et al., Current Protocols in Molecular Biology, John Wiley & Sons, New York, N.Y., 1994). Once produced, monoclonal antibodies are also tested for specific tumor antigen recognition by Western blot or immunoprecipitation analysis (by the methods described in F. Ausubel et al., supra).

Monoclonal and polyclonal antibodies that specifically recognize a tumor antigen (or fragments thereof), such as those described herein, are considered useful in the invention. For example, antibodies that specifically recognize a tumor antigen may be used to measure the level of that tumor antigen in a patient sample (such as blood). An increased level of the antigen, relative to a reference sample from a normal subject without a tumor, may indicate the presence of a tumor in a patient undergoing testing. Furthermore, an increased level of antigen, relative to an earlier sample taken from the same patient, may indicate an increase in tumor burden (i.e., metastasis).

Antibodies of the invention may be produced using tumor antigen amino acid sequences that do not reside within highly conserved regions, and that appear likely to be antigenic, as analyzed by criteria such as those provided by the Peptide Structure Program (Genetics Computer Group Sequence Analysis Package, Program Manual for the GCG Package, Version 7, 1991) using the algorithm of Jameson and Wolf (CABIOS 4:181, 1988). These fragments can be generated by standard techniques, e.g., by the PCR, and cloned into the pGEX expression vector (F. Ausubel et al., Current Protocols in Molecular Biology, John Wiley & Sons, New York, N.Y., 1994). GST fusion proteins are expressed in E. coli and purified using a glutathione agarose affinity matrix as described in F. Ausubel et al., supra). To generate rabbit polyclonal antibodies, and to minimize the potential for obtaining antisera that is non-specific, or exhibits low-affinity binding to a tumor antigen, two or three fusions are generated for each protein, and each fusion is injected into at least two rabbits. Antisera are raised by injections in series, preferably including at least three booster injections.

In addition, antibodies of the invention may be produced using tumor antigen amino acid sequences that do reside within highly conserved regions. These antibodies may be screened as described above.

In addition to intact monoclonal and polyclonal anti-tumor antigen antibodies, the invention features various genetically engineered antibodies, humanized antibodies, and antibody fragments, including F(ab′)2, Fab′, Fab, Fv and sFv fragments. Antibodies can be humanized by methods known in the art, e.g., monoclonal antibodies with a desired binding specificity can be commercially humanized (Scotgene, Scotland; Oxford Molecular, Palo Alto, Calif.). Fully human antibodies, such as those expressed in transgenic animals, are also features of the invention (Green et al., Nature Genetics 7:13-21, 1994).

Ladner (U.S. Pat. Nos. 4,946,778 and 4,704,692) describes methods for preparing single polypeptide chain antibodies. Ward et al. (Nature 341:544-546, 1989) describe the preparation of heavy chain variable domains, termed “single domain antibodies,” which have high antigen-binding affinities. McCafferty et al. (Nature 348:552-554, 1990) show that complete antibody V domains can be displayed on the surface of fd bacteriophage, that the phage bind specifically to antigen, and that rare phage (one in a million) can be isolated after affinity chromatography. Boss et al. (U.S. Pat. No. 4,816,397) describe various methods for producing immunoglobulins, and immunologically functional fragments thereof, which include at least the variable domains of the heavy and light chain in a single host cell. Cabilly et al. (U.S. Pat. No. 4,816,567) describe methods for preparing chimeric antibodies.

Use of Anti-Tumor Antigen Antibodies

Antibodies specific for tumor antigens may be used, as noted above, to detect tumor antigens in a patient sample (such as blood, a tumor biopsy, or other biological material obtained from a patient) or to inhibit the biological activities of tumor antigens. For example, nucleic acid encoding an antibody or portion of an antibody may be expressed within a cell to inhibit tumor antigen function.

In addition, the antibodies may be coupled to compounds for diagnostic and/or therapeutic uses. For example, the antibodies may be coupled to imaging compounds, such as radionucleotides, for imaging a tumor. Imaging methods are known to those skilled in the art (e.g., radiologists and nuclear physicians); they include, and are not limited to, X-rays, computerized tomography (CT) scans, magnetic resonance imaging (MRI), positron emission tomography (PET) scans, scintography, single photon emission computerized tomography (SPECT) scans, nuclear medicine scanning methods in general, and analogous approaches that allow detection and visualization of a tumor. Antibodies may also be coupled to radionuclides or toxic compounds (such as ricin) for specifically targeting anti-tumor therapy to a tumor, or to liposomes containing compounds for anti-tumor therapy.

Detection of Tumor Antigen Gene Expression

As noted, the antibodies described above may be used to monitor tumor antigen protein expression. In addition, in situ hybridization may be used to detect the expression of tumor antigen genes. In situ hybridization techniques, such as fluorescent in situ hybridization (FISH), rely upon the hybridization of a specifically labeled nucleic acid probe to the cellular RNA in individual cells or tissues. Therefore, such techniques allow the identification of mRNA within intact tissues, such as a patient biopsy. In this method, oligonucleotides or cloned nucleic acid (RNA or DNA) fragments corresponding to unique portions of tumor antigen genes are used to detect specific mRNA species that are differentially expressed in tumor vs. normal tissue. Numerous other gene expression detection techniques are known to those of skill in the art and may be employed within the methods of the invention.

Detection of Altered Expression Levels of Tumor Antigens and Mutations in Tumor Antigens

Tumor antigen polypeptides and nucleic acid sequences find diagnostic use in the detection or monitoring of tumorigenesis and metastasis. Accordingly, an increase in the level of tumor antigen may indicate the development of a tumor or metastasis. Levels of tumor antigen may be assayed by any standard technique. Tumor antigen expression in a biological sample (e.g., a biopsy) may be monitored by standard Northern blot analysis or may be aided by PCR (see, e.g., F. Ausubel et al., Current Protocols in Molecular Biology, John Wiley & Sons, New York, N.Y., 1994; PCR Technology: Principles and Applications for DNA Amplification, H. A. Ehrlich, Ed., Stockton Press, NY; Yap et al. Nucl. Acids. Res. 19:4294, 1991).

A biological sample obtained from a patient may be analyzed for one or more mutations in tumor antigen nucleic acid sequences using a mismatch detection approach. Generally, these techniques involve PCR amplification of nucleic acid from the patient sample, followed by identification of the mutation (i.e., mismatch) by either altered hybridization, aberrant electrophoretic gel migration, binding or cleavage mediated by mismatch binding proteins, or direct nucleic acid sequencing. Any of these techniques may be used to facilitate mutant tumor antigen detection, and each is well known in the art; examples of particular techniques are described, without limitation, in Orita et al. (Proc. Natl. Acad. Sci. USA 86:2766-2770, 1989) and Sheffield et al. (Proc. Natl. Acad. Sci. USA 86:232-236, 1989).

Mismatch detection assays also provide an opportunity to diagnose a predisposition to developing a tumor before the onset of symptoms. For example, a patient heterozygous for a mutation in a tumor antigen gene may show no clinical symptoms and yet possess a higher than normal probability of developing cancer. Given this diagnosis, a patient may take precautions to minimize their exposure to adverse environmental factors (for example, excessive exposure to ultraviolet light) and to carefully monitor their medical condition (for example, through frequent physical examinations). The tumor antigen diagnostic assays described above may be carried out using any appropriate biological sample (for example, any biopsy sample, blood sample, or other tissue or body fluid sample that contains nucleic acid and/or protein).

Alternatively, a tumor antigen mutation, particularly as part of a diagnosis for predisposition to cancers associated with expression of a specific tumor antigen, may be tested using a DNA sample from any cell, for example, by mismatch detection techniques. Preferably, the DNA sample is subjected to PCR amplification prior to analysis.

In yet another approach, immunoassays are used to detect or monitor tumor antigen expression in a biological sample. Tumor antigen-specific polyclonal or monoclonal antibodies (produced as described above) may be used in any standard immunoassay format (e.g., enzyme-linked immunosorbent assay (ELISA), Western blot, or radioimmunoassay (RIA)) to measure tumor antigen polypeptide levels. These levels are compared to reference (wild-type) tumor antigen levels. For example, an increase in a tumor antigen, relative to reference levels, may indicate the presence of a tumor. Examples of immunoassays are described, e.g., in Ausubel et al., Current Protocols in Molecular Biology, John Wiley & Sons, New York, N.Y., 1994 through 1998.

Immunohistochemical techniques may also be utilized for tumor antigen detection. For example, a tumor biopsy may be obtained from a patient, sectioned, and stained for the presence of tumor antigens using an anti-tumor antigen antibody and any standard detection system (e.g., one which includes a secondary antibody conjugated to horseradish peroxidase). General guidance regarding such techniques may be found in, e.g., Bancroft and Stevens (Theory and Practice of Histological Techniques, Churchill Livingstone, 1982) and Ausubel et al. (Current Protocols in Molecular Biology, John Wiley & Sons, New York, N.Y., 1994 through 1998). Detection of a particular tumor antigen within the presence of a tumor may provide information concerning tumor prognosis and approaches to treatment.

Vaccination with Tumor Antigens for Tumor Therapy or Prophylaxis

Vaccination protocols may be designed to induce or enhance an immune response against a tumor antigen in order to treat an existing cancer, or prevent the development or recurrence of cancer. Reagents that are useful for vaccination may include, without limitation, full length tumor antigen polypeptides, or fragments thereof, or nucleic acids that encode tumor antigens (e.g., a viral or plasmid expression vector that carries a tumor antigen gene) or tumor antigen fragments.

a) Vaccination with Tumor Antigen Polypeptides

In order to produce a vaccination against one or more tumor antigens, it is necessary to obtain large amounts of pure tumor antigen protein from eukaryotic or prokaryotic cultured cells that express the protein. This can be achieved by methods that are described above and are known in the art. Induction of an immune response by administration of a tumor antigen protein to a subject may be achieved by conventional techniques that are well-known to those skilled in the art of vaccine production and delivery.

b) Vaccination by Gene Therapy

Gene therapy is another approach that may be used to induce an immune response in a subject to be treated for, or protected against, cancer. A tumor antigen-encoding gene, or a portion thereof, must be delivered to cells in a form that can be taken up and express sufficient protein to induce an effective immune response.

Transducing retroviral, adenoviral, and human immunodeficiency viral (HIV) vectors are suited for somatic cell gene therapy, because they show high efficiency of infection and stable integration and expression; see, e.g., Cayouette, M., and Gravel, C., (1997) Hum. Gene Therapy, 8:423-430; Kido, M., et al. (1996) Curr. Eye Res., 15:833-844; Bloomer, U., et al. (1997) J. Virol., 71:6641-6649; Naldini, L., et al. (1996) Science 272:263-267; Miyoshi, H., et al. (1997), Proc. Nat. Acad. Sci., U.S.A., 94:10319-1032. For example, a full length tumor antigen gene, or portions thereof, can be cloned into a retroviral vector and transcribed via its endogenous promoter, or via the retroviral long terminal repeat, or via a promoter specific for the target cell type of interest. Other viral vectors that can be used include adenovirus, adeno-associated virus, vaccinia virus, bovine papilloma virus, or a herpes virus such as Epstein-Barr Virus.

Gene transfer in vivo may also be achieved by non-viral means. For example, viral or plasmid vectors encoding tumor antigens or fragments thereof may be injected directly into skeletal muscle or cardiac muscle by previously described methods (e.g., Wolff, J. A., et al., Science, 247:1465-1468, 1990). Expression vectors injected into skeletal muscle in situ are taken up into muscle cell nuclei and used as templates for expression of their encoded proteins. Tumor antigen genes that are engineered to contain a signal peptide are secreted from tumor antigen-expressing muscle cells, after which they induce an immune response. Gene transfer into cells within the tissues of a living animal also may be achieved by lipofection (Felgner et al., Proc. Natl. Acad. Sci. USA 84: 7413, 1987; Ono et al., Neurosci. Lett. 117: 259, 1990; Brigham et al., Am. J. Med. Sci. 298:278, 1989; Staubinger et al., Meth. Enz. 101:512, 1983), or asialoorosomucoid-polylysine conjugation (Wu et al., J. Biol. Chem. 263:14621, 1988; Wu et al., J. Biol. Chem. 264:16985, 1989).

Retroviral vectors, adenoviral vectors, adenovirus-associated viral vectors, or other viral vectors also may be used to deliver tumor antigen genes to cells ex vivo. Numerous vectors useful for this purpose are generally known (Miller, Human Gene Therapy 15-14, 1990; Friedman, Science 244:1275-1281, 1989; Eglitis and Anderson, BioTechniques 6:608-614, 1988; Tolstoshev and Anderson, Curr. Opin. Biotech. 1:55-61, 1990; Sharp, The Lancet 337: 1277-1278, 1991; Cornetta et al., Nucl. Acid Res. and Mol. Biol. 36: 311-322, 1987; Anderson, Science 226: 401-409, 1984; Moen, Blood Cells 17: 407-416, 1991; Miller et al., Biotech. 7: 980-990, 1989; Le Gal La Salle et al., Science 259: 988-990, 1993; and Johnson, Chest 107: 77S-83S, 1995). Retroviral vectors are particularly well developed and have been used in clinical settings (Rosenberg et al., N. Engl. J. Med 323: 370, 1990; Anderson et al., U.S. Pat. No. 5,399,346).

Gene transfer into cells ex vivo can also be achieved by delivery of non-viral vectors, such as expression plasmids, using methods such as calcium phosphate or DEAE dextran transfection, electroporation, and protoplast fusion. Liposomes may also be potentially beneficial for delivery of DNA into a cell.

Cells that are to be transduced or transfected ex vivo may be obtained from a patient (e.g., bone marrow stem cells or cells from a tumor biopsy) prior to transfection, and re-introduced after transfection. However, the cells also may be derived from a source other than the patient undergoing gene transfer.

In the constructs described above, tumor antigen mRNA expression can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in skeletal muscle cells may be used to direct tumor antigen gene expression for vaccination in situ. The enhancers used may include, without limitation, those that are characterized as tissue- or cell-specific in their expression. Alternatively, if a tumor antigen genomic clone is used as a therapeutic construct, regulation may be mediated by the cognate regulatory sequences or, if desired, by regulatory sequences derived from a heterologous source, including any of the promoters or regulatory elements described above.

Gene Therapy Approaches for Inhibiting Tumor Antigen Function

As described above for MAIAP, antisense-based strategies may be employed to explore tumor antigen gene function. Moreover, inhibition of tumor antigen function via antisense gene therapy may, in some cases, may provide an effective anti-tumor therapeutic approach.

The principle of antisense therapy is based on the hypothesis that sequence-specific suppression of gene expression (via transcription or translation) may be achieved by intracellular hybridization between genomic DNA or mRNA and a complementary antisense species. The formation of such a hybrid nucleic acid duplex interferes with transcription of the target tumor antigen-encoding genomic DNA, or processing/transport/translation and/or stability of the target tumor antigen mRNA.

Antisense nucleic acids may be delivered by a variety of approaches. For example, antisense oligonucleotides or antisense RNA may be directly administered (e.g., by intravenous injection) to a subject in a form that allows uptake into cells (e.g., tumor cells). Alternatively, viral or plasmid vectors that encode antisense RNA (or RNA fragments) may be introduced into cells in vivo or ex vivo. Antisense effects can be induced by sense sequences, however, the extent of phenotypic changes are highly variable. Phenotypic changes induced by effective antisense therapy are assessed according to changes in, e.g., protein levels, protein activity measurement, and target mRNA levels.

In a specific example, inhibition of tumor antigen function by antisense gene therapy may be accomplished by direct administration of antisense tumor antigen mRNA to a subject. The antisense tumor antigen mRNA may be produced and isolated by any standard technique, but is most readily produced by in vitro transcription using an antisense tumor antigen cDNA under the control of a high efficiency promoter (e.g., the T7 promoter). Administration of antisense tumor antigen mRNA to cells can be carried out by any of the methods for direct nucleic acid administration described above.

An alternative strategy for inhibiting tumor antigen function using gene therapy involves intracellular expression of an anti-tumor antigen antibody or a portion of an anti-tumor antigen antibody. For example, the gene (or gene fragment) encoding a monoclonal antibody that specifically binds to tumor antigen and inhibits its biological activity may be placed under the transcriptional control of a specific (e.g., tissue- or tumor-specific) gene regulatory sequence.

Administration of Tumor Antigen Polypeptides or Nucleic Acids that Comprise Tumor Antigen-Encoding or -Antisense Sequences

Conventional pharmaceutical practice may be employed to provide suitable formulations or compositions to administer tumor antigen vaccinations or antisense nucleic acids for treatment of, or prophylaxis against, cancer. Tumor antigen polypeptides or fragments thereof, genes (or antisense nucleic acids) or fragments thereof, or tumor antigen-specific antibodies, may be administered within a pharmaceutically-acceptable diluent, carrier, or excipient, in unit dosage form. Administration may begin before a patient is symptomatic. Any appropriate route of administration may be employed, for example, administration may be parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration. Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in Remington's Pharmaceutical Sciences, (18^{th} edition), ed. A. Gennaro, 1990, Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for tumor antigens and tumor antigen-inhibitory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Vaccination with Irradiated, Autologous Melanoma Cells Engineered to Secrete Human GM-CSF Generates Potent Anti-Tumor Immunity in Patients with Metastatic Melanoma

### General Methods

Clinical Protocol

The details of the clinical study design and methods of vaccine production have been presented previously (Soiffer et al., Hum. Gene Ther., 8:111-123, 1997; Ellem et al., Cancer Immunol. Immunother, 44:10-20, 1997; Simone et al., Cancer Res. 57:1537-1546, 1997.). In brief, surgically resected tumors were processed to single-cell suspension by collagenase and mechanical digestion and introduced into short-term culture. Replicating tumor cells were transduced with viral supernatants harvested from CRIP packaging cell lines transfected with MFG-S-human GM-CSF, irradiated with 15,000 cGy, and cryopreserved in liquid nitrogen. Transduced cells were certified to be free of replication-competent retrovirus (RCR), endotoxin, mycoplasma, and other microbial contaminants. GM-CSF secretion was determined by ELISA (R&D). A portion of the tumor culture for use in delayed-type hypersensitivity evaluation was irradiated but not transduced. Frozen cells were thawed and washed in HBSS prior to injection; vaccines were administered intradermally (0.5 ml) and subcutaneously (0.5 ml) into normal skin on the limbs and abdomen on a rotating basis. Non-transduced cells were injected intradermally (0.5 ml) into normal skin at the time of beginning vaccination and then at monthly intervals in order to measure the generation of delayed-type hypersensitivity. Patient sera were tested regularly for RCR; all samples were negative.

Immunologic Analyses and Histopathology

Peripheral blood mononuclear cells were obtained by centrifugation over Ficoll gradients. Tumor infiltrating lymphocytes were prepared by mechanical digestion of metastatic deposits. For cytokine assays, lymphocytes were co-cultured with irradiated, autologous melanoma cells in 24 well dishes in 2 mls of DME plus 10% fetal calf serum, antibiotics, 2-mercaptoethanol, and glutamine. Media was harvested at day eight and assayed for IL-3, IL-4, IL-5, IL-6, IL-10, GM-CSF, γ-IFN, TNF-α, and TNF-β production by ELISA using the appropriate monoclonal antibodies (Pharmingen). For cytotoxicity assays, lymphocytes were bulk stimulated with irradiated, autologous tumor cells for one week in media plus 10 U/ml IL-2 and then tested using standard techniques against ⁵¹Cr labeled tumor targets (Kruisbeek et al., Current Protocols in Immunology, John Wiley & Sons, 1991).

For immunoblotting analysis, 0.5-2.0 mg of melanoma cell lysates (phosphate buffered saline supplemented with 0.5% NP-40, soybean trypsin inhibitor, leupeptin, pepstatin, aminocaproic acid, and PMSF) were electrophoresed on SDS-polyacrylamide 4-12% gradient gels, transferred to Immobilon membranes (Millipore), and blocked overnight at room temperature in 5% non-fat dry milk in PBS. Membranes were probed overnight at 4° C. in a 1:100 dilution of patient sera (in Tween 20/Tris buffered saline), washed, and incubated for one hour at room temperature with an anti-human IgG, Fcγ-specific antibody conjugated to alkaline phosphatase (Jackson Immuno Research). The membranes were then developed with NBT and BCIP (Promega).

For flow cytometry analysis, at least 100,000 melanoma cells were incubated with a 1:100 dilution of patient serum (in 1% non-fat milk) for three hours on ice, washed, and then stained with a 1:100 dilution of anti-human IgG, Fcγ-specific antibody conjugated to FITC. Lymphocytes were phenotyped with standard techniques using monoclonal antibodies against CD3, CD4, CD8, CD14, CD19, CD45RA, CD45RO, and CD56 (Coulter).

For histopathology, tissues were formalin-fixed, paraffin-embedded, and stained with hematoxylin and eosin.

### Results

Patient Population, Vaccine Production, and Vaccine Administration

Thirty-three metastatic melanoma patients (stage 1V) ranging from 32 to 82 years of age (18 women, 15 men) were enrolled in the clinical protocol (Soiffer et al., Hum. Gene Ther., 8:111-123, 1997). Twenty had received prior therapies. Patients underwent a surgical procedure to remove a metastatic lesion for vaccine preparation. Tumors were harvested from soft tissue (14 patients), lymph node (9 patients), lung (6 patients), liver (3 patients), and adrenal gland (1 patient). Resected lesions were processed by mechanical and enzymatic digestion into single-cell suspensions and introduced into short-term culture. Proliferating melanoma cells were transduced with a replication-defective retrovirus expressing GM-CSF, irradiated with 15,000 cGy (which induced cell cycle arrest, but did not inhibit secretion of CSF by cultured cells for at least seven days), and cryopreserved.

Two patients were excluded from the study after enrollment because of the absence of melanoma in the surgical specimen and two were excluded because vaccines could not be produced. In the remaining twenty-nine patients, vaccines were successfully generated, achieving GM-CSF secretion rates ranging from 84 to 965 ng per 10⁶ cells per 24 hours. The duration of vaccine preparation was generally 8 weeks (the range was 8-32 weeks). Three successive patient cohorts were immunized intradermally and subcutaneously with 10⁷ irradiated tumor cells (each treatment) administrated at 28, 14, or 7 day intervals (dose levels 1, 2, and 3, respectively) for a total of 84 days (total of 3, 6, or 12 vaccinations). Four patients at dose level 3 received additional vaccinations (up to a total of 24) after the first course of therapy. Three patients were withdrawn from the study after tumor harvest and prior to vaccination because of rapid disease progression. Five patients were withdrawn from study after beginning vaccination because rapid disease progression prevented administration of the full course of immunizations. Twenty-one patients were withdrawn from study after beginning vaccination because rapid disease progression prevented administration of the full course of immunizations. Twenty-one patients completed therapy (three at dose level 1, four at dose level 2, and fourteen at dose level 3), were fully evaluable for toxicity and biologic activity, and comprised the study population reported here. Sites of metastatic disease were skin, subcutaneous tissue, lymph node, lung, liver, spleen, intestine, adrenal, kidney, and bone. The number of different organ systems involved with metastases was: one (8 patients), two (13 patients), three (7 patients), four (4 patients), and six (1 patient). Twenty patients received prior systemic therapies including IL-2, a-interferon, IL-12, IL-1, monoclonal antibody, BCG, tumor vaccine, and chemotherapy (DTIC, BCNU, taxol, cisplatin, carboplatin, vinblastine, fotomustine, cyclophophamide, and tamoxifen).

Toxicities

Vaccination elicited erythema and induration at injection sites. Reactions were associated with local pruritus that was easily controlled with emollients. Grade 1 fatigue and nasal congestion were occasionally noted. No hepatic, renal, pulmonary, cardiac, hematologic, gastrointestinal, or neurologic toxicities were observed. No patient experienced vitiligo or autoimmune events.

Vaccination Reactions

Injections of irradiated, autologous, GM-CSF secreting melanoma cells evoked striking local reactions in all patients, with the intensity and duration of the responses generally increasing in proportion to the number of vaccines administered. Clinically, the reactions were characterized by substantial erythema (up to 35 cm in diameter) and induration (up to 14 cm in diameter). Occasionally, the reactions became hemorrhagic. Vaccination responses tended to peak at approximately 48 hours following cell injection, but the elicited induration could persist for several weeks, particularly after multiple immunizations. An intriguing observation was the frequent development of recall reactions at sites of previous vaccination. Several patients continued to experience these reactions intermittently in a mild form even after completion of therapy (for up to two years), although no clear precipitants were identified.

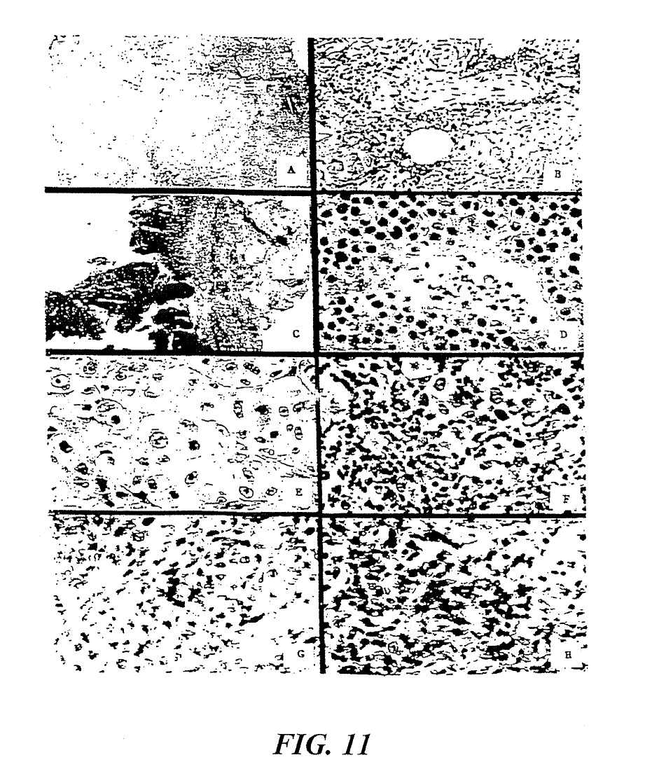

Vaccination sites in all patients were characterized histologically by an extensive infiltrate of dendritic cells, macrophages, eosinophils, and T lymphocytes which extended throughout the dermis and into the subcutaneous fat (FIG. 11A, injection site of irradiated GM-CSF-secreting melanoma cells following vaccination; note the extensive inflammatory reaction throughout all layers of the skin and the marked fibrosis in the subcutaneous fat). The infiltrates at dose levels 2 and 3 were usually more cellular than those at dose level 1 and also more frequently resulted in the development of flame figures (collections of deposited eosinophil granules) and endothelial cell damage in the superficial venules of the upper dermis. Eosinophil degranulation in nerve sheaths, lymphocytic infiltration of hair follicles, and fat necrosis were observed in several patients as well.

Delayed-Type Hypersensitivity Reactions

Although injections of irradiated, autologous, non-transfected melanoma cells failed to elicit significant responses in all patients at the time of early treatment, these injections evoked strong responses in all patients after several vaccinations were administered. Clinically, these delayed-type hypersensitivity reactions were characterized by extensive erythema (up to 10 cm) and induration (up to 6 cm) which peaked at 48 hours and then gradually resolved. Histopathologically, the reactions were characterized by dense infiltrates of T lymphocytes and degranulating eosinophils extending throughout the dermis (FIG. 11B, injection site of irradiated, non-transfected melanoma cells following vaccination). The infiltrates at dose levels 2 and 3 were usually greater than those at dose level 1.

Eosinophilia

In addition to the striking involvement of eosinophils in the reactions to injections of both irradiated, GM-CSF secreting and irradiated, non-transfected melanoma cells, significant increases in the numbers of peripheral blood eosinophils (but not other leukocytes) were also observed following immunization, with mean peak eosinophil counts of 705±715, 515±102, and 928±571 per mm³ for dose levels 1, 2, and 3 respectively. The duration of eosinophilia tended to vary as a function of dose, with elevated counts persisting for several weeks more frequently at dose level 3 than at dose levels 1 or 2.

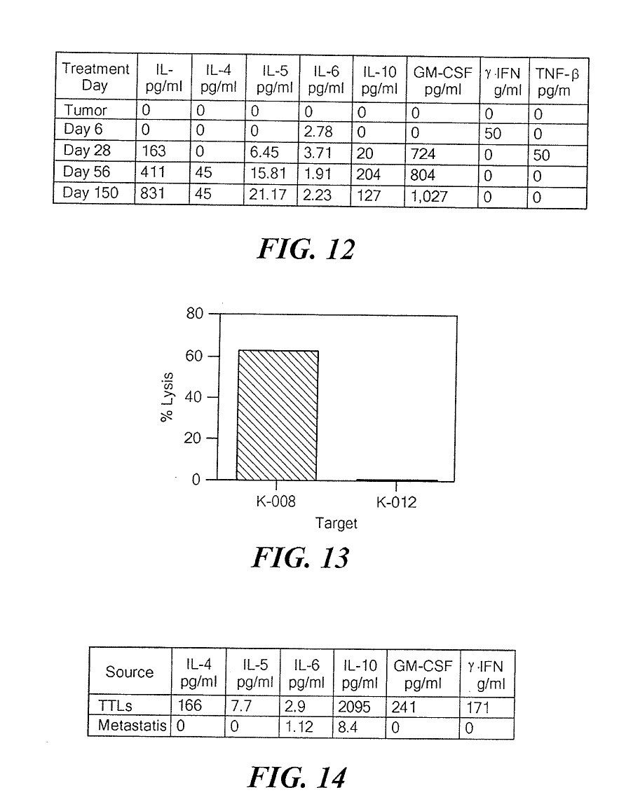

Since eosinophilia in many model systems is T cell-dependent, we investigated whether vaccination induced alterations in peripheral blood T cell cytokine production. For these studies, 1×10⁶ peripheral blood mononuclear cells, obtained at various times during treatment, were cultured with 1×10⁴ autologous, irradiated, non-transfected melanoma cells in the absence of supplemental growth factors; culture supernatants were harvested at day eight and assayed for cytokine content by ELISA (FIG. 12; “Tumor” represents the cytokines produced by the autologous, irradiated, non-transfected melanoma cells in the absence of lymphocytes). Vaccinations were administered on days 0, 28, and 56. In nine of ten patients studied, vaccination elicited substantial levels of T cell-derived IL-5, IL-3, and GM-CSF, in contrast to the variable production of IL-4, IL-6, IL-10, and TNF-β and the negligible induction of γ-interferon. The enhanced T cell secretion of IL-3, IL-5, and GM-CSF as a consequence of vaccination likely contributed, at least in part, to the augmented production of eosinophils, as these molecules have been shown to enhance the proliferation of eosinophilic precursors in vitro and in vivo. Moreover, the persistence of distinctive cytokine profiles for several months after completing treatment suggests that immunization stimulated the development of memory T cells.

Immune Responses in Metastases

To determine whether vaccination generated anti-melanoma immune responses capable of inducing anti-tumor effects, we examined the host reactions to metastatic lesions resected prior to and after completing therapy. Metastatic lesions procured before the beginning of immunization revealed in all patients either the absence of host reactivity or only a modest inflammatory reaction present focally within the tumor (FIG. 11E). Metastatic lesions resected after the completion of immunization, however, demonstrated a profound immune response in 11 of 16 patients from which tissue could be obtained (FIG. 11C; note extensive necrosis and fibrosis). These responses were found in metastatic lesions (up to 10 cm in diameter) derived from a variety of sites including skin, subcutaneous tissue, lymph node, lung, spleen, and intestine.

One important characteristic of the anti-melanoma immune reaction in each of the 11 responding patients was the diffuse infiltration of tumor masses by large numbers of T lymphocytes and plasma cells (FIG. 11F). Many CD4 and CD8 positive T lymphocytes were organized into rosettes around dying melanoma cells (satellitosis), a morphologic pattern indicative of lymphocyte-induced tumor apoptosis (FIG. 11G shows CD4-positive T cell reaction in metastasis following vaccination; FIG. 11H shows CD8-positive T cell reaction in metastasis following vaccination; tissues were formalin-fixed, paraffin-embedded, and stained with hematoxylin/eosin and anti-CD4 or anti-CD8). Plasma cells accounted for nearly 50% of the inflammatory cells and were intimately associated with the T lymphocytes and melanoma cells.

A second intriguing feature of the anti-melanoma response, observed in 4 patients, was the targeted destruction of the tumor vasculature, whereby lymphocytes, eosinophils, and neutrophils were closely associated with dying tumor blood vessels (FIG. 11D). Overall, the chronic inflammatory reactions evident in these 11 patients resulted in substantial tumor destruction (at least 80%) and the development of significant edema and fibrosis throughout the resected metastases. Of the five patients failing to develop inflammatory infiltrates in metastatic lesions as a consequence of vaccination, two were treated at dose level 1 and three had rapidly progressive disease resulting in death shortly after completion of therapy. No significant differences in the metastatic immune responses were observed between dose levels 2 and 3.

Characterization of Anti-Melanoma Cellular and Humoral Immunity

The functional properties of the lymphocytes infiltrating the metastatic lesions were examined in two patients and found to be comparable. When single-cell suspensions of the excised metastases were introduced into culture in the presence of low doses of IL-2 (10 U/ml), the inflammatory cells lysed all of the residual, viable melanoma cells. To quantify cytotoxicity in a more formal way, we established additional primary cultures of the explanted metastases in which the non-adherent, inflammatory cells were removed; viable melanoma cells could be propagated in this way. Briefly, tumor infiltrating lymphocytes obtained from an abdominal wall metastasis removed following vaccination were cultured for one week with autologous melanoma cells (derived from the resected metastasis) in the presence of 10 U/ml IL-2. A four hour ⁵¹Cr release assay against the autologous tumor (K008) and a second melanoma line (effector:target ratio of 50:1) derived from another patient (K012, differing MHC class I profile) was performed. When the tumor infiltrating lymphocytes (bulk-cultured for one week with the autologous melanoma cells plus 10 U/ml IL-2) were tested in a standard 4 hour cytotoxicity assay against the explanted melanoma cells, highly significant lysis of the autologous melanoma cells, but not melanoma cells derived from another patient (differing MHC profile), was observed (FIG. 13).

The tumor infiltrating lymphocytes also demonstrated the ability to produce a broad range of cytokines in response to the autologous melanoma cells, a property which likely contributed to the enhanced T cell cytotoxicity and the prominent anti-tumor plasma cell response. ELISA analysis of the conditioned medium obtained by co-culturing the tumor infiltrating lymphocytes and autologous melanoma cells for one week in the presence of 10 U/ml of IL-2 revealed substantial levels of IL-4, IL-5, IL-6, IL-10, GM-CSF, and γ-interferon, but not TNF-β (FIG. 14; “Metastasis” refers to tumor cells cultured alone; similar results were found with a second patient examined). This cytokine profile indicates the coordinate expression of gene products which are associated with both Th1 and Th2 cells and suggests that multiple lymphocyte effector mechanisms can result in potent anti-tumor immune responses. Moreover, the substantial secretion of IL-10 is provocative, given the widely held view that this molecule is primarily immunosuppressive.

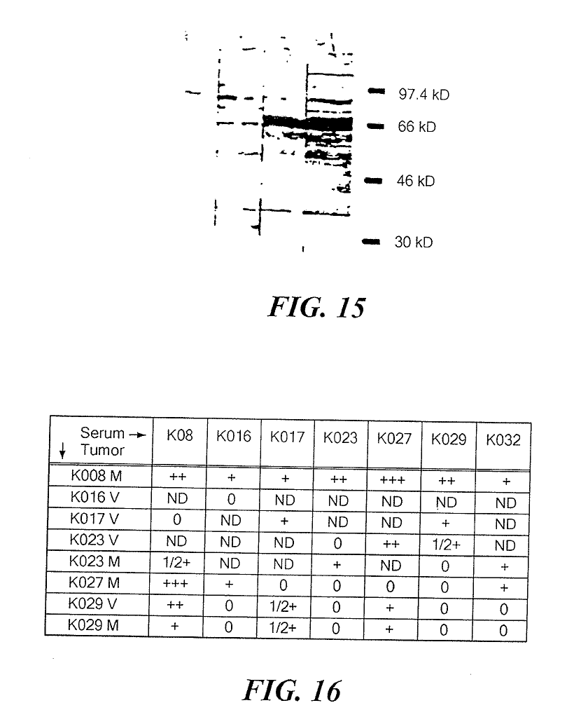

To determine whether the plasma cell infiltration of the metastatic lesions resulted in the generation of antibodies recognizing melanoma cells, we performed immunoblotting analysis using autologous melanoma cell lysates and sera obtained at various times during vaccination. Melanoma cell lysates were electrophoresed on 4-12% SDS-polyacrylamide gradient gels and immunoblotted with 1:100 dilutions of autologous serum obtained at various times during treatment. Membranes were developed with an alkaline phosphatase-conjugated anti-human IgG, Fcγ-specific antibody. Immunization stimulated the enhanced production of IgG anti-melanoma antibodies in seven patients examined thus far (FIG. 15; A, pre-treatment; B, one month after starting vaccination; C, two months after starting vaccination; D, three months after starting vaccination; similar results were obtained with six additional patients; increased reactivity was specific for autologous cells, as testing of allogeneic cell lysates revealed different patterns of reactivity). The reactivity of post-vaccination sera was characterized both by increased recognition of proteins detected by pre-immunization sera and the recognition of proteins not detected by pre-immunization sera.

The induction of IgG antibodies recognizing surface melanoma cell determinants was also demonstrated in these patients by flow cytometry analysis. Briefly, melanoma cell lines were stained with 1:100 dilutions of sera obtained before and after vaccination, developed with an anti-human IgG, Fcγ-specific antibody, and analyzed by flow cytometry. Changes in reactivity as a function of vaccination are reported in FIG. 16 (“M,” cell line established from a metastasis removed following treatment; “V,” cell line established from a metastasis used to prepare the vaccine; +++, strong shift between pre- and post-immunization sera; ++, intermediate shift; +, small shift; ½+, borderline shift; 0, no shift; ND, not determined). Significant augmentation of reactivity to cultured melanoma cells as a function of vaccination was observed (FIG. 16). The specificity profiles of the antibodies elicited by immunization suggest the existence of several independent antigens.

Clinical Outcome

According to standard clinical criteria, one partial response (shrinkage of subcutaneous lesions), one mixed response, and three minor responses were observed. Three patients remain free of disease with follow-up of 33, 33, and 17 months, respectively; two were rendered disease-free by surgery (pathologic examination showed brisk lymphocyte and plasma cell infiltration with extensive tumor necrosis); one underwent radiation therapy to a scapular metastasis during vaccine preparation. Prior to beginning immunization, these patients had developed multiple new metastatic lesions.

Isolation of Melanoma Antigen-Encoding Clones Using Phage Expression Libraries

Library Construction

Polyadenylated mRNA is isolated using guanidinium isothiocyanate phenol-chloroform extraction in the presence of β-mercaptoethanol (Chomczynski and Sacchi, Anal. Chem., 162:156-159, 1987) followed by oligo(dT)-cellulose chromatography (Stratagene). Expression libraries are constructed using the lambda-derived unidirectional cloning vector UniZap according to procedures developed by the manufacturer (Stratagene). In brief, mRNA is reverse-transcribed with Moloney murine leukemia virus reverse transcriptase and 5-methyl dCTP using an oligo(dT) linker primer containing an Xho I site. The 5-methyl dCTP leads to methylation of the first strand, protecting it from digestion with Xho I. To generate the second cDNA strand, RNase H is used to nick the RNA strand; these breaks then serve as primers for DNA polymerase I to nick-translate the second strand of the cDNA. For the second strand synthesis dCTP (un-methylated) is used, so that the Xho I sites in the linker are accessible for digestion. The cDNA is then blunted with Pfu DNA polymerase and EcoR I adapters are ligated. The adapters are phosphorylated only on the blunt side to minimize their annealing to one another. A kinase reaction is then performed on the ligated adaptors so that the cDNA will be able to be cloned into the vector. Xho I digestion is carried out, resulting in fragments with 5′ EcoR I and 3′ Xho I ends. The cDNA is size fractionated on a Sephacryl S-500 column (we use fragments of 1200 base pairs and larger). The fractionated cDNA is then ligated into the UniZap vector and packaged into viral particles using Gigapack III gold extracts. The vector is initially propagated in XL1-Blue MRF′ bacteria which are McrA⁻, McrB⁻ and which therefore will not restrict the hemimethylated DNA. It will be appreciated, by those of skill in the art, that analogous methods for isolating polyadenylated mRNA and constructing expression libraries may be used.

Isolation of Tumor Antigen Clones by Expression Library Screening Using Antibodies

For immunologic screening, the closed insert is expressed as a fusion protein in E. coli using the beta-galactosidase promoter in the vector. The episome in XL1-Blue MRF′ bacteria contains the lac repressor, which blocks transcription from the lacZ promoter in the absence of the inducer IPTG (isopropyl-1-thio-β-D-galactopyranoside) and thereby prevents potential toxicity of some inserts which might result in the inhibition of phage replication. Plated phage (5×10⁴ plaques per 150-cm dish) are propagated at 42° C. for 3.5 hours and then induced with IPTG treated membranes (Schleicher and Schuell) for four hours at 37° C. Replicate lifts are prepared. Membranes are then washed extensively with TTBS and blocked overnight in 5% w/v non-fat dry milk in PBS. Membranes are screened at room temperature overnight with various dilutions of patient post-vaccination serum in TTBS (to date we have tested 1:500, 1:1500, and 1:5000 with equivalent results). The serum is extensively pre-absorbed against lambda lysate of E. coli. The membranes are again extensively washed and then probed with a 1:1000 dilution of alkaline phosphatase conjugated anti human IgG Fc-specific (Jackson) which has also been extensively preabsorbed against lambda lysate of E. coli. Membranes are then developed with NBT and BCIP (Promega). Positive plaques are purified through secondary and tertiary screenings.

pBluescript phagemid can be removed from the UniZap vector by infection with a helper phage that allows excision to occur (Short et al., Nuc. Acids Res. 16:7583-7600, 1988; Alting-Mees et al., Meth. Enzymol. 216:483-495, 1992). Since this helper phage cannot replicate in a nonsuppressing host (SOLR cells) due to the presence of an amber mutation, simple purification of the excised phagemid is possible. The excised phagemids are then sequenced with T7 and T3 primers according to the instructions provided for the Sequenase kit. Sequences are compared against those in sequence databases using the BLAST and BEAUTY programs to determine whether they are related to or identical to known genes (Altschul et al., J. Mol. Biol., 215:403-410, 1991; Worley et al., Genome Res., 5:173-184, 1995). Full length cDNA sequences are then obtained using RACE (Clontech) to amplify the 5′ and 3′ ends (Frohman et al., Proc. Natl. Acad. Sci. U.S.A., 85:8998-9002, 1988).

The expression profiles of melanoma antigens are assessed by Northern analysis or RT-PCR using a panel of melanoma tumor lines, cloned melanocytes (obtained from Clontech), various tumor tissues, and normal tissue obtained from autopsy material. The sequence of the normal counterpart is determined to delineate whether any mutation is associated with the tumor. For Northern blot analysis, 10 μg of total RNA prepared by guanidinium-isothiocyanate phenol extraction (TRIZOL, Gibco BRL) is electrophoresed in a 1% agarose formaldehyde gel and transferred to Zetabind membranes. We use Stratagene QuikHyb for hybridization in a hybridization oven at 65° C. Membranes are probed with a cDNA probe and β-actin probe labeled with ³²P to high specific activity by the random hexamer method. Membranes are then washed twice with 2× standard saline citrate (SSC)/0.1% SDS at 60° C. for 15 minutes and then once with 0.1×SSC at 60° C. for 30 minutes. The membranes are developed by autoradiography. For RT-PCR analysis, 2 μg of total RNA is reverse-transcribed in a total volume of 20 μl with 4 μl of 5× reverse transcriptase buffer (Gibco BRL), 2 μl of a 20 mM solution of oligo (dT) primers, 20 U of RNasin (Promega), 2 μl of 0.1 M dithiothreitol, and 200 U of MoMLV reverse transcriptase (Giobco BRL). After heat killing the enzyme, 1 μl of 0.1 M RNase H (Pharmacia) is added and the reaction performed for 20 minutes at 37° C. One twentieth of the sample is then used in PCR reactions as follows: 5.0 μl 10×PCR buffer (350 mM KCl, 9 mM MgCl₂, 0.01% gelatin), 1 μl dNTPs, 1 μl 5′ primer, 1 μl 3′ primer, 0.5 μl Taq DNA polymerase, water to 50 μl. Typically, such PCR reactions are incubated for 30 cycles of 30 sec. at 95° C., 60 sec. at 55° C., and 2 minutes at 72° C.

Use of this screening method has allowed us to isolate clones encoding the tumor antigens described above, i.e., TRAAM, TPR/UBP3, UBP3, BRAP-2/H-ATPase, KOO8-1, MAIAP, Gene AS, BR-1, BR-2, KIAA0603, TPR, NOR-90, and BRAP-2.

Isolation of Tumor Antigen Clones by Expression Library Screening Using Cytotoxic T Cells

In addition to antibody-based approaches for identifying novel tumor antigens revealed by vaccination with autologous, GM-CSF-secreting tumor cells, patient cytotoxic T lymphocytes (CTLs) may be used to isolate tumor antigen-encoding clones from expression libraries, using the cloning strategy developed by Boon et al. (Ann. Rev. Immunol. 12:337-365, 1994).

CTL clones may be obtained by the protocol of Herin et al. (Int. J. Cancer, 39:390-396, 1987). In brief, 1×10⁶ tumor infiltrating lymphocytes are cultured in 24 well dishes with 1×10⁵ irradiated, autologous melanoma cells in 2 ml of culture medium (DME plus 10 mM HEPES buffer, 10% fetal calf serum, 2 mM glutamine, 2×10⁻⁵ M β-mercaptoethanol, minimal essential amino acids and antibiotics). On day three, 25 U/ml IL-2 is added and on day seven, cultures are re-stimulated. This process is repeated for four weeks, after which responding lymphocytes are transferred in 200 μl of medium to 96 well dishes at 1 cell per well together with 3000 irradiated autologous melanoma cells and 105 irradiated allogeneic EBV-immortalized blasts as feeders. T cell functional responses evoked in response to tumor cells, such as cytotoxicity, proliferation, or cytokine production (such as GM-CSF and TNF-α) is then evaluated; such assays are well-known in the art.

Plasmid DNA from mammalian expression libraries cloned into the vector pCDM8 is divided into multiple pools and introduced into COS cells by DEAE-dextran precipitation, along with an expression plasmid encoding the relevant MHC class I molecule. The transfected COS cells are incubated for 48 hours at 37° C. and then co-cultured with lymphocytes plus IL-2. 24 hours later, tumor-specific T cell functional responses such as cytotoxicity, proliferation, or cytokine production (such as GM-CSF and TNF-α) are evaluated. DNA is extracted from transfected COS cells inducing responses from CTLs and the procedure is repeated until individual cDNA clones are obtained. The cDNA inserts are sequenced and further analyzed as described above.

The T cell antigenicity of tumor antigens isolated by the antibody screening method may be determined using the T cell functional assays described above. Similarly, tumor antigens identified by cytotoxic T cell-based screens may be tested for their humoral immunogenicity by testing the reactivity of patient sera to such antigens.

Determination of Tumor Antigen Immunogenicity

The relative immunogenicity of a tumor antigen cloned by the method of the invention may be tested by sub-cloning the tumor antigen-encoding cDNA into an expression vector (e.g., plasmid or viral), introducing the cDNA-bearing vector into a suitable cell line (e.g., by transfection, electroporation, or transduction), making lysates from the tumor antigen-expressing cells, and subjecting the lysates to Western blot analysis or ELISA using sera from patients vaccinated with the tumor antigen or with whole tumor cells.

Construction of Recombinant Retroviral Vectors Expressing Tumor Antigens

To clone cDNA sequences into pMFG (Dranoff et al., Proc. Natl. Acad. Sci., U.S.A., 90:3539-3543, 1993), we construct oligonucleotides encompassing the ATG of the insert and adapt it into the vector Nco I site (unless a natural Nco I or BspHI site exists at the ATG of the cDNA). We then clone the remainder of the insert into the BamH I site of the pMFG vector in such a way so as to include as little sequence downstream of the stop codon as practical. After the integrity of the final construct is confirmed by sequencing, the plasmid is co-transfected by calcium phosphate precipitation with pSV2NEO into CRIP cells as previously described (Danos et al., Proc. Natl. Acad. Sci. U.S.A. 85:6460-6464, 1988). G418 selection is instituted at 36 hours, resistant clones picked, expanded, and eventually titered on NIH 3T3 cells using Southern analysis. The infected CRIP tumor cells are tested for the presence of helper virus using a very sensitive hisD mobilization assay (Hartman et al., Proc. Natl. Acad. Sci. U.S.A. 85:8047-8051, 1988). The method involves the construction of an indicator NIH 3T3 cell line transfected with a packageable retrovirus encoding the hisD gene (histidinol dehydrogenase from Salmonella typhimurium). Mammalian cells, including NIH 3T3 fibroblasts, when grown in histidine-free medium supplemented with histidinol, die from the combined effects of histidine deficiency and the inhibitory activities of histidinol on histidyl-tRNA synthetase. The expression of hisD, which catalyzes the dehydrogenation of histidinol to histidine, rescues transfected cells from this toxicity. Medium from the infected tumor cells is placed on the hisD transfected indicator cells. The medium is then harvested from the exposed hisD transfected cells and then placed on control NIH3T3 cells. The generation of hisD resistant cells in this control population is assessed. If helper virus is present in the infected tumor lines being tested, the retroviral vector encoding hisD is transferred to the control NIH 3 T3 cells, giving rise to cells that are resistant when grown under histidinol selection.

Measurement of Antibody Responses

The development of antibody responses in patients vaccinated with tumor antigens ro with whole tumor cells may be assessed by Western analysis of CRIP cell lysates or other mammalian cell lines. Comparable dilutions of sera are evaluated; if reactivity is present pre-immunization, additional dilutions of post-vaccination sera may be examined in an attempt to perform semi-quantitative analysis. For immunoblotting, we prepare cell lysates as follows. Adherent cells are rinsed in PBS and then lysed in 300 μl of lysate buffer (138 mM NaCl, mM NaH₂PO₄, 1.5 mM KH₂PO₄, 2.7 mM KCl, pH 7.2, supplemented with 0.05 M aminocaproic acid, 100 μg/ml soybean trypsin inhibitor, 10 μg/ml leupeptin, 1 μg/ml pepstatin, 0.5% Nonidet P-40) per 10 cm dish. A Bradford assay is performed to determine protein content (Bradford, Anal. Biochem., 72:248-254, 1976). Prior to gel electrophoresis, the lysates are preabsorbed with normal sera. To do this, 100 μl of a 50% Protein A Sepharose bead suspension (Pharmacia) is washed with PBS and lysate buffer twice. 20 μl of normal human serum and 100 μl of lysate buffer are added to the beads on ice for 30 minutes and then the beads are washed four times with lysate buffer. Protein lysates from control CRIP cells and transfected CRIP lines are incubated with the beads at 4° C. overnight with gentle shaking. The samples are then centrifuged and the supernatants harvested.

The proteins (500-1000 μg per sample) are resolved on a 12.5% SDS-polyacrylamide gel under reducing conditions. The samples are transferred to Immobilon membranes via semi-dry electrophoretic transfer in CAPS buffer. The membranes are blocked overnight (5% w/v non-fat dry milk in PBS), washed twice in TTBS (50 mM Tris (Tris-hydroxymethyl-aminomethane), 138 mM NaCl, 2.7 mM KCl, 0.05% w/v Tween 20, pH 8.0), and then probed with patient serum (1:50 dilution) in TTBS at 4° C. overnight. The membrane is then washed with TTBS and incubated for one hour at room temperature with a 1:1000 dilution in TTBS of an anti-human IgG, Fc gamma-specific antibody conjugated to alkaline phosphatase (Jackson Immuno Research). Lastly, the membrane is extensively washed with TTBS and then developed with nitroblue tetrazolium (NBT) and 5-bromo-4-chloro-3-indolyl phosphate (BCIP) (Promega).

### Other Embodiments

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth, and follows in the scope of the appended claims.

1-15. (canceled)

16. An isolated TRAAM polypeptide, wherein said polypeptide is encoded by a nucleic acid sequence selected from the group consisting of:
TRAAM (SEQ ID NO: 17); and NOR-90 (SEQ ID NO: 13).

17. (canceled)

18. An isolated TRAAM nucleic acid, wherein said nucleic acid encodes the TRAAM polypeptide set forth in SEQ ID NOs: 18 or 19.

19. An isolated TRAAM nucleic acid, wherein said nucleic acid comprises a nucleotide sequence set forth in SEQ ID NO: 17.

20. An isolated nucleic acid comprising a probe, wherein said probe hybridizes under high stringency conditions to TRAAM (SEQ ID NO: 17) and wherein said probe has a nucleotide sequence complementary to at least 14 nucleotides of TRAAM.

21. The nucleic acid of claim 18 or 20, wherein said nucleic acid is DNA or RNA.

22. A vector comprising the nucleic acid of claim 18.

23. An isolated cell comprising the nucleic acid of claim 18.

24. An isolated antibody that specifically binds a polypeptide encoded by a nucleic acid sequence selected from the group consisting of chosen from:
TRAAM (SEQ ID NO: 17) and NOR-90 (SEQ ID NO: 13).

25. A method of generating an antibody that specifically binds a polypeptide or a fragment thereof encoded by a nucleic acid sequence selected from the group consisting of:
TRAAM (SEQ ID NO: 17) and NOR-90 (SEQ ID NO: 13) said method comprising administering said polypeptide, or said fragment thereof, to an animal capable of generating an immune response, and isolating said antibody from said animal.

26. A method of detecting the presence of a polypeptide, or a fragment thereof, in a biological sample, wherein said fragment comprises at least 10 amino acids, and wherein said polypeptide or fragment is encoded by a nucleic acid selected from the group consisting of:
TRAAM (SEQ ID NO: 17) and NOR-90 (SEQ ID NO: 13),
said method comprising contacting said sample with an antibody that specifically binds said polypeptide or a fragment thereof, and assaying for binding of said antibody to said polypeptide.

27. A method of testing a patient for the presence of a tumor or an increased likelihood of developing a tumor, said method comprising:
a) obtaining a sample from said patient;
b) measuring the level of an antibody in said sample, wherein said antibody specifically binds a tumor antigen, wherein said tumor antigen comprises a polypeptide encoded by a nucleic acid selected from the group consisting of: TRAAM (SEQ ID NO: 17); and NOR-90 (SEQ ID NO: 13);
c) comparing the antibody level in the patient sample to the antibody level in a reference sample, wherein an increase in said antibody level in said patient sample, relative to said antibody level in said reference sample, indicates that said patient has a tumor or the increased likelihood of developing a tumor.

28. A method of testing a patient for the presence of a tumor or an increased likelihood of developing a tumor, said method comprising:
a) obtaining a sample from said patient;
b) measuring the level of cytotoxic T lymphocytes in said sample, wherein said cytotoxic T lymphocytes specifically bind a tumor antigen, wherein said tumor antigen comprises a polypeptide encoded by a nucleic acid selected from the group consisting of: TRAAM (SEQ ID NO: 17); and NOR-90(SEQ ID NO: 13)
c) comparing the cytotoxic T lymphocyte level in the patient sample to the cytotoxic T lymphocyte level in a reference sample, wherein an increase in said cytotoxic T lymphocyte level in said patient sample, relative to said cytotoxic T lymphocyte level in said reference sample, indicates that said patient ha a tumor or the increased likelihood of developing a tumor.

29. The method of claim 27 or 28, wherein said tumor is a leukemia, a lymphoma, a brain tumor, a melanoma, a fibrosarcoma, or a uterine, cervical, testicular, liver, ovarian, lung, renal cell, colon, breast, prostate, or bladder carcinoma.

30. A method of testing a patient for the presence of a tumor or the increased likelihood of developing a tumor, said method comprising:
a) obtaining a sample from said patient,
b) measuring the level of a tumor antigen in said sample, wherein said tumor antigen comprises a polypeptide encoded by a nucleic acid selected from the group consisting of: TRAAM (SEQ ID NO: 17); NOR-90 (SEQ ID NO: 13),
c) comparing the tumor antigen level in the patient sample to the tumor antigen level in a reference sample, wherein an increase in said tumor antigen level in said patient sample, relative to said tumor antigen level in said reference sample, indicates that said patient has a tumor or the increased likelihood of developing a tumor.

31. The method of claim 30, wherein said tumor antigen level is measured by measuring the level of tumor antigen polypeptide.

32. The method of claim 30, wherein said tumor antigen level is measured by measuring the level of nucleic acid encoding said tumor antigen.

33. The method of claim 32, wherein said nucleic acid is genomic DNA.

34. The method of claim 32, wherein said nucleic acid is mRNA.

35. The method of claim 32, wherein said nucleic acid is cDNA.

36. The method of claim 30, wherein said sample is selected from: a tumor or tissue biopsy, a lymph node, bone marrow, cells, blood, urine, stool, sputum, saliva, cerebrospinal fluid, or uterine tissue.

37. The method of claim 30, wherein said tumor is a leukemia, a lymphoma, a brain tumor (e.g., a neuroblastoma), a melanoma, a fibrosarcoma, or a carcinoma such as a uterine, cervical, testicular, liver, ovarian, lung (e.g., non-small cell lung), renal cell, colon, breast, prostate, or bladder carcinoma.

38. A method of determining the level of an antibody in a patient, wherein said antibody specifically binds a tumor antigen polypeptide comprising a polypeptide encoded by a nucleic acid selected from the group consisting of:
TRAAM (SEQ ID NO: 17); and NOR-90 (SEQ ID NO: 13), said method comprising:
a) obtaining a sample containing said antibody from said patient, and
b) measuring the level of said antibody in the patient sample, compared to a reference sample.

39. A method of treatment of prophylaxis for a patient that has a tumor or is at risk for developing a tumor, said method comprising vaccinating said patient with a tumor antigen, or a fragment thereof, wherein said fragment comprises at least 10 amino acids, wherein said tumor antigen is encoded by a nucleic acid selected from the group consisting of:
TRAAM (SEQ ID NO: 17); NOR-90 (SEQ ID NO: 13).

40. The method of claim 39, wherein said vaccinating is with a tumor antigen polypeptide.

41. The method of claim 39, wherein said vaccinating is with a nucleic acid encoding said tumor antigen polypeptide or said fragment thereof, wherein said nucleic acid is operably linked to a promoter.

42. The method of claim 41, wherein said nucleic acid is within an expression vector.

43. The method of claim 41, wherein said nucleic acid is within a cell capable of expressing said nucleic acid.

44. The method of claim 43, wherein said nucleic acid is introduced into said cell in vivo.

45. The method of claim 43, wherein said nucleic acid is introduced into said call ex vivo.

46. A method for treating a tumor in a patient, said method comprising administering to said patient, an antibody that specifically binds a tumor antigen encoded by a nucleic acid selected from the group consisting of:
TRAAM (SEQ ID NO: 17) and NOR-90 (SEQ ID NO: 13); and NOR-90 (SEQ ID NO: 13).

47. The method of claim 46, wherein said antibody is coupled to a toxic or radioactive moiety.

48. A method for detecting a tumor in a patient, said method comprising:
a) introducing, into said patient, an antibody coupled to an imaging compound, wherein said antibody specifically binds a tumor antigen encoded by a nucleic acid selected from the group consisting of:
TRAAM (SEQ ID NO: 17) and NOR-90 (SEQ ID NO: 13); and NOR-90 (SEQ ID NO: 13), and
b) detecting immune complexes formed between said antibody and said tumor antigen in said patient.

49. A vaccine for treatment of a tumor or prophylaxis against developing a tumor, said vaccine comprising a substantially pure tumor antigen polypeptide or a fragment thereof, wherein said fragment comprises at least 10 amino acids, wherein said tumor antigen polypeptide comprises of polypeptide encoded by a nucleic acid selected from the group consisting of:
TRAAM (SEQ ID NO 17); and NOR-90; and BRAP-2.

50. A vaccine for treatment of a tumor or prophylaxis against developing a tumor, comprising a substantially pure nucleic acid encoding a tumor antigen or a fragment thereof, wherein said fragment comprises at least 10 amino acids, wherein said tumor antigen is encoded by a nucleic acid selected from the group consisting of:
TRAAM (SEQ ID NO: 17) and NOR-90 (SEQ ID NO: 13); and NOR-90 (SEQ ID NO: 13).

51. The method of claim 50, wherein said nucleic acid is within a cell, wherein said nucleic acid is expressed in said cell.

52. The vaccine of claim 50, wherein said nucleic acid is within a vector.

53-73. (canceled)

74. (canceled)

75. An isolated TRAAM polypeptide, wherein said polypeptide comprises the partial TRAM repeat sequence set forth in SEQ ID NO: 25.

76. An isolated TRAAM polypeptide, wherein said polypeptide comprises the full TRAM repeat sequence set forth in SEQ ID NO: 24.

77. An isolated TRAAM polypeptide, wherein said polypeptide comprises the PSET repeat sequence set forth in SEQ ID NO: 30.

78. An isolated NOR-90 nucleic acid, encoded by NOR-90 nucleic acid encodes a TRAAM polypeptide substantially identical to the polypeptide set forth in SEQ ID NO: 13.

79. The TRAAM nucleic acid of claim 18, wherein said nucleic acid comprises the nucleotide sequence set forth in SEQ ID NO: 17.

80. A substantially pure nucleic acid that comprises at least 14 consecutive nucleotides that display at least 85%, 90%, 92%, 95% or 98% sequence identity to a nucleotide sequence that is complementary to a nucleic acid that encodes a TRAAM polypeptide (SEQ ID NO: 18 or 19).

81. The substantially pure nucleic acid of claim 80, wherein said nucleic acid comprises at least 16, 18, 22, 25, 50, 75, or 100 consecutive nucleotides that display at least 85%, 90%, 92%, 95% or 98% sequence identity to a nucleotide sequence that is complementary to a nucleic acid that encodes a TRAAM polypeptide (SEQ ID NO: 18 or 19).

82. The nucleic acid of claim 80, wherein said nucleic acid hybridizes under high stringency conditions to a TRAAM nucleic acid.

83. A substantially pure nucleic acid comprising at least 14 nucleotides, wherein said nucleic acid hybridizes under high stringency conditions to a nucleic acid that encodes a TRAAM polypeptide (SEQ ID NO: 18 or 19).

84. The substantially pure nucleic acid of claim 83, wherein said substantially pure nucleic acid comprises at least 16, 18, 22, 25, 50, 75, or 100 nucleotides.

85. The substantially pure nucleic acid of claim 80 or 83, wherein said substantially pure nucleic acid is an antisense nucleic acid.
